SEQUENCE LISTING (124 sequences)

SEQ ID NO: 1 - protein, 13 aa, Artificial sequence
  Synthetic polypeptide
KNGGPSSGAPPPS

SEQ ID NO: 2 - protein, 9 aa, Artificial sequence
  Synthetic polypeptide
PSSGAPPPS

SEQ ID NO: 3 - protein, 18 aa, Artificial sequence
  Synthetic polypeptide
FIEWLKNGGPSSGAPPPS

SEQ ID NO: 4 - protein, 7 aa, Artificial sequence
  Synthetic polypeptide
PKKIRYS

SEQ ID NO: 5 - protein, 39 aa, Heloderma horridum
HSDGTFTSDLSKQMEEEAVRLFIEWLKNGGPSSGAPPPS

SEQ ID NO: 6 - protein, 39 aa, Heloderma suspectum
HGEGTFTSDLSKQMEEEAVRLFIEWLKNGGPSSGAPPPS

SEQ ID NO: 7 - protein, 39 aa, Artificial sequence
  Synthetic polypeptide
HGEGTFTSDLSKQLEEEAVRLFIEWLKNGGPSSGAPPPS

SEQ ID NO: 8 - protein, 37 aa, Artificial sequence
  Synthetic polypeptide
HGEGTFTSDLSKQLEEEAVRLFIEWLKQGGPSKEIIS

SEQ ID NO: 9 - protein, 31 aa, Homo sapiens
HAEGTFTSDVSSYLEGQAAKEFIAWLVKGRG

SEQ ID NO: 10 - protein, 30 aa, Artificial sequence
  Synthetic polypeptide
XXXGXXXXXXXXXXXXXAXXXXXXXXXXGG

SEQ ID NO: 11 - protein, 31 aa, Artificial sequence
  Synthetic polypeptide
XXXGXXXXXXXXXXXXXAXXXXXXXXXXGGX

SEQ ID NO: 12 - protein, 32 aa, Artificial sequence
  Synthetic polypeptide
XXXGXXXXXXXXXXXXXAXXXXXXXXXXGGXS

SEQ ID NO: 13 - protein, 33 aa, Artificial sequence
  Synthetic polypeptide
XXXGXXXXXXXXXXXXXAXXXXXXXXXXGGXSS

SEQ ID NO: 14 - protein, 34 aa, Artificial sequence
  Synthetic polypeptide
XXXGXXXXXXXXXXXXXAXXXXXXXXXXGGXSSG

SEQ ID NO: 15 - protein, 35 aa, Artificial sequence
  Synthetic polypeptide
XXXGXXXXXXXXXXXXXAXXXXXXXXXXGGXSSGA

SEQ ID NO: 16 - protein, 36 aa, Artificial sequence
  Synthetic polypeptide
XXXGXXXXXXXXXXXXXAXXXXXXXXXXGGXSSGAX

SEQ ID NO: 17 - protein, 37 aa, Artificial sequence
  Synthetic polypeptide
XXXGXXXXXXXXXXXXXAXXXXXXXXXXGGXSSGAXX

SEQ ID NO: 18 - protein, 38 aa, Artificial sequence
  Synthetic polypeptide
XXXGXXXXXXXXXXXXXAXXXXXXXXXXGGXSSGAXXX

SEQ ID NO: 19 - protein, 44 aa, Artificial sequence
  Synthetic polypeptide
HGEGTFTSDLSKQMEEEAVRLFIEWLKNGGPSSGAPPSKKKKKK

SEQ ID NO: 20 - protein, 50 aa, Artificial sequence
  Synthetic polypeptide
KKKKKKHGEGTFTSDLSKQMEEEAVRLFIEWLKNGGPSSGAPPSKKKKKK

SEQ ID NO: 21 - protein, 38 aa, Artificial sequence
  Synthetic polypeptide
HGEGTFTSDLSKQMEEEAVRLFIEWLWLKNGGPSSGAS

SEQ ID NO: 22 - protein, 42 aa, Artificial sequence
  Synthetic polypeptide
KKKKKKHGEGTFTSDLSKQMEEEAVRLFIEWLKNGGPSSGAS

SEQ ID NO: 23 - protein, 42 aa, Artificial sequence
  Synthetic polypeptide
NEEEEEHGEGTFTSDLSKQMEEEAVRLFIEWLKNGGPSSGAS

SEQ ID NO: 24 - protein, 42 aa, Artificial sequence
  Synthetic polypeptide
HGEGTFTSDLSKQMEEEAVRLFIEWLKNGGPSSGASKKKKKK

SEQ ID NO: 25 - protein, 48 aa, Artificial sequence
  Synthetic polypeptide
KKKKKKHGEGTFTSDLSKQMEEEAVRLFIEWLKNGGPSSGASKKKKKK

SEQ ID NO: 26 - protein, 48 aa, Artificial sequence
  Synthetic polypeptide
DEEEEEHGEGTFTSDLSKQMEEEAVRLFIEWLKNGGPSSGASKKKKKK

SEQ ID NO: 27 - protein, 6 aa, Artificial sequence
  Synthetic polypeptide
KKKKKK

SEQ ID NO: 28 - protein, 37 aa, Mus musculus
KCNTATCATQRLANFLVRSSNNLGPVLPPTNVGSNTY

SEQ ID NO: 29 - protein, 37 aa, Homo sapiens [sequence omitted]

SEQ ID NO: 30 - protein, 37 aa, Artificial sequence
  Synthetic polypeptide
KCNTATCATQRLANFLVHSSNNFGPILPPTNVGSNTY

SEQ ID NO: 31 - protein, 37 aa, Artificial sequence
  Synthetic polypeptide
XCNTATCATQRLANFLVHSSXNFXXXXXXTXVGSNTY

SEQ ID NO: 32 - protein, 32 aa, Homo sapiens
CGNLSTCMLGTYTQDFNKFHTFPQTAIGVGAP

SEQ ID NO: 33 - protein, 32 aa, Salmo salar
CSNLSTCVLGKLSQELHKLQTYPRTNTGSGTP

SEQ ID NO: 34 - protein, 32 aa, Artificial sequence
  Synthetic polypeptide
KCNTATCVLGRLSQELHRLQTYPRTNTGSNTY

SEQ ID NO: 35 - protein, 4 aa, Artificial sequence
  Synthetic polypeptide
GGGG

SEQ ID NO: 36 - protein, 5 aa, Artificial sequence
  Synthetic polypeptide
GGGGG

SEQ ID NO: 37 - protein, 6 aa, Artificial sequence
  Synthetic polypeptide
GGGGGG

SEQ ID NO: 38 - protein, 7 aa, Artificial sequence
  Synthetic polypeptide
GGGGGGG

SEQ ID NO: 39 - protein, 5 aa, Artificial sequence
  Synthetic polypeptide
GGGGS

SEQ ID NO: 40 - protein, 8 aa, Artificial sequence
  Synthetic polypeptide
GAGAGAGA

SEQ ID NO: 41 - protein, 8 aa, Artificial sequence
  Synthetic polypeptide
AAAAAAAA

SEQ ID NO: 42 - protein, 5 aa, Artificial sequence
  Synthetic polypeptide
GGGGK

SEQ ID NO: 43 - protein, 6 aa, Artificial sequence
  Synthetic polypeptide
GGGGKR

SEQ ID NO: 44 - protein, 8 aa, Artificial sequence
  Synthetic polypeptide
GGGKGGGG

SEQ ID NO: 45 - protein, 8 aa, Artificial sequence
  Synthetic polypeptide
GGGNGSGG

SEQ ID NO: 46 - protein, 8 aa, Artificial sequence
  Synthetic polypeptide
GGGCGGGG

SEQ ID NO: 47 - protein, 5 aa, Artificial sequence
  Synthetic polypeptide
GPNGG

SEQ ID NO: 48 - protein, 10 aa, Artificial sequence
  Synthetic polypeptide
GGGGSGGGGS

SEQ ID NO: 49 - protein, 25 aa, Artificial sequence
  Synthetic polypeptide
GGGGSGGGGSGGGGSGGGGSGGGGS

SEQ ID NO: 50 - protein, 6 aa, Artificial sequence
  Synthetic polypeptide
GGEGGG

SEQ ID NO: 51 - protein, 8 aa, Artificial sequence
  Synthetic polypeptide
GGEEEGGG

SEQ ID NO: 52 - protein, 5 aa, Artificial sequence
  Synthetic polypeptide
GEEEG

SEQ ID NO: 53 - protein, 4 aa, Artificial sequence
  Synthetic polypeptide
GEEE

SEQ ID NO: 54 - protein, 6 aa, Artificial sequence
  Synthetic polypeptide
GGDGGG

SEQ ID NO: 55 - protein, 7 aa, Artificial sequence
  Synthetic polypeptide
GGDDDGG

SEQ ID NO: 56 - protein, 5 aa, Artificial sequence
  Synthetic polypeptide
GDDDG

SEQ ID NO: 57 - protein, 4 aa, Artificial sequence
  Synthetic polypeptide
GDDD

SEQ ID NO: 58 - protein, 21 aa, Artificial sequence
  Synthetic polypeptide
GGGGSDDSDEGSDGEDGGGGS

SEQ ID NO: 59 - protein, 5 aa, Artificial sequence
  Synthetic polypeptide
WEWEW

SEQ ID NO: 60 - protein, 5 aa, Artificial sequence
  Synthetic polypeptide
FEFEF

SEQ ID NO: 61 - protein, 6 aa, Artificial sequence
  Synthetic polypeptide
EEEWWW

SEQ ID NO: 62 - protein, 6 aa, Artificial sequence
  Synthetic polypeptide
EEEFFF

SEQ ID NO: 63 - protein, 7 aa, Artificial sequence
  Synthetic polypeptide
WWEEEWW

SEQ ID NO: 64 - protein, 7 aa, Artificial sequence
  Synthetic polypeptide
FFEEEFF

SEQ ID NO: 65 - protein, 22 aa, Artificial sequence
  Synthetic polypeptide
GSGSATGGSGSTASSGSGSATH

SEQ ID NO: 66 - protein, 22 aa, Artificial sequence
  Synthetic polypeptide
HGSGSATGGSGSTASSGSGSAT

SEQ ID NO: 67 - protein, 19 aa, Artificial sequence
  Synthetic polypeptide
AEAAAKEAAAKEAAAKAGG

SEQ ID NO: 68 - protein, 28 aa, Artificial sequence
  Synthetic polypeptide
HGEGTFTSDLSKQMEEEAVRLFIEWLKN

SEQ ID NO: 69 - protein, 31 aa, Artificial sequence
  Synthetic polypeptide
CNTATCVLGRLSQELHRLQTYPRTNTGSNTY

SEQ ID NO: 70 - protein, 40 aa, Artificial sequence
  Synthetic polypeptide
HGEGTFTSDLSKQLEEEAVRLFIEWLKNGGPSSGAPPPSK

SEQ ID NO: 71 - protein, 62 aa, Artificial sequence
  Synthetic polypeptide
HGEGTFTSDLSKQMEEEAVRLFIEWLKNXXKCNTATCVLGRLSQELHRLQTYPRTNTGSNTY

SEQ ID NO: 72 - protein, 72 aa, Artificial sequence
  Synthetic polypeptide
HGEGTFTSDLSKQLEEEAVRLFIEWLKQGGPSKEIISGGGKCNTATCVLGRLSQELHRLQTYPRTNTGSNTY

SEQ ID NO: 73 - protein, 37 aa, Artificial sequence; identical to SEQ ID NO: 8
  Synthetic polypeptide

SEQ ID NO: 74 - protein, 74 aa, Artificial sequence
  Synthetic polypeptide
HGEGTFTSDLSKQLEEEAVRLFIEWLKNGGPSSGAPPPSGCGKCNTATCVLGRLSQELHRLQTYPRTNTGSNTY

SEQ ID NO: 75 - protein, 36 aa, Mus musculus
CNTATCATQRLANFLVRSSNNLGPVLPPTNVGSNTY

SEQ ID NO: 76 - protein, 37 aa, Artificial sequence
  Synthetic polypeptide
KCNTATCATQRLANFLVRSSKNLGPVLPPTNVGSNTY

SEQ ID NO: 77 - protein, 36 aa, Artificial sequence
  Synthetic polypeptide
CNTATCATQRLANFLVRSSKNLGPVLPPTNVGSNTY

SEQ ID NO: 78 - protein, 37 aa, Artificial sequence
  Synthetic polypeptide
KCNTATCATQRLANFLVRSSNNLGPKLPPTNVGSNTY

SEQ ID NO: 79 - protein, 36 aa, Artificial sequence
  Synthetic polypeptide
CNTATCATQRLANFLVRSSNNLGPKLPPTNVGSNTY

SEQ ID NO: 80 - protein, 37 aa, Artificial sequence
  Synthetic polypeptide
KCNTATCATQRLANFLVRSSNNLGPVLPPTKVGSNTY

SEQ ID NO: 81 - protein, 36 aa, Artificial sequence
  Synthetic polypeptide
CNTATCATQRLANFLVRSSNNLGPVLPPTKVGSNTY

SEQ ID NO: 82 - protein, 37 aa, Artificial sequence; identical to SEQ ID NO: 30
  Synthetic polypeptide

SEQ ID NO: 83 - protein, 36 aa, Artificial sequence
  Synthetic polypeptide
CNTATCATQRLANFLVHSSNNFGPILPPTNVGSNTY

SEQ ID NO: 84 - protein, 36 aa, Artificial sequence
  Synthetic polypeptide
CNTATCATQRLANFLVHSSKNFGPILPPTNVGSNTY

SEQ ID NO: 85 - protein, 36 aa, Artificial sequence
  Synthetic polypeptide
CNTATCATQRLANFLVHSSNNFGPKLPPTNVGSNTY

SEQ ID NO: 86 - protein, 36 aa, Artificial sequence
  Synthetic polypeptide
CNTATCATQRLANFLVHSSNNFGPILPPTKVGSNTY

SEQ ID NO: 87 - protein, 36 aa, Artificial sequence
  Synthetic polypeptide
CNTATCATQRLANFLVHSSNNFKPILPPTNVGSNTY

SEQ ID NO: 88 - protein, 36 aa, Artificial sequence
  Synthetic polypeptide
CNTATCATQRLANFLVHSSNNFGKILPPTNVGSNTY

SEQ ID NO: 89 - protein, 36 aa, Artificial sequence
  Synthetic polypeptide
CNTATCATQRLANFLVHSSNNFGPIKPPTNVGSNTY

SEQ ID NO: 90 - protein, 36 aa, Artificial sequence
  Synthetic polypeptide
CNTATCATQRLANFLVHSSNNFGPILKPTNVGSNTY

SEQ ID NO: 91 - protein, 36 aa, Artificial sequence
  Synthetic polypeptide
CNTATCATQRLANFLVHSSNNFGPILPKTNVGSNTY

SEQ ID NO: 92 - protein, 37 aa, Artificial sequence; identical to SEQ ID NO: 28
  Synthetic polypeptide

SEQ ID NO: 93 - protein, 36 aa, Artificial sequence; identical to SEQ ID NO: 75
  Synthetic polypeptide

SEQ ID NO: 94 - protein, 37 aa, Artificial sequence; identical to SEQ ID NO: 76
  Synthetic polypeptide

SEQ ID NO: 95 - protein, 36 aa, Artificial sequence; identical to SEQ ID NO: 77
  Synthetic polypeptide

SEQ ID NO: 96 - protein, 37 aa, Artificial sequence; identical to SEQ ID NO: 78
  Synthetic polypeptide

SEQ ID NO: 97 - protein, 36 aa, Artificial sequence; identical to SEQ ID NO: 79
  Synthetic polypeptide

SEQ ID NO: 98 - protein, 37 aa, Artificial sequence; identical to SEQ ID NO: 80
  Synthetic polypeptide

SEQ ID NO: 99 - protein, 36 aa, Artificial sequence; identical to SEQ ID NO: 81
  Synthetic polypeptide

SEQ ID NO: 100 - protein, 37 aa, Artificial sequence; identical to SEQ ID NO: 78
  Synthetic polypeptide

SEQ ID NO: 101 - protein, 36 aa, Artificial sequence; identical to SEQ ID NO: 84
  Synthetic polypeptide

SEQ ID NO: 102 - protein, 36 aa, Artificial sequence; identical to SEQ ID NO: 85
  Synthetic polypeptide

SEQ ID NO: 103 - protein, 36 aa, Artificial sequence; identical to SEQ ID NO: 86
  Synthetic polypeptide

SEQ ID NO: 104 - protein, 36 aa, Artificial sequence; identical to SEQ ID NO: 85
  Synthetic polypeptide

SEQ ID NO: 105 - protein, 36 aa, Artificial sequence; identical to SEQ ID NO: 87
  Synthetic polypeptide

SEQ ID NO: 106 - protein, 36 aa, Artificial sequence; identical to SEQ ID NO: 88
  Synthetic polypeptide

SEQ ID NO: 107 - protein, 36 aa, Artificial sequence; identical to SEQ ID NO: 89
  Synthetic polypeptide

SEQ ID NO: 108 - protein, 36 aa, Artificial sequence; identical to SEQ ID NO: 90
  Synthetic polypeptide

SEQ ID NO: 109 - protein, 36 aa, Artificial sequence; identical to SEQ ID NO: 91
  Synthetic polypeptide

SEQ ID NO: 110 - protein, 36 aa, Artificial sequence
  Synthetic polypeptide
CNTATCATQRLANFLVHSSNKFGPILPPTNVGSNTY

SEQ ID NO: 111 - protein, 36 aa, Artificial sequence
  Synthetic polypeptide
CNTATCATQRLANFLVHSSNNKGPILPPTNVGSNTY

SEQ ID NO: 112 - protein, 36 aa, Artificial sequence
  Synthetic polypeptide
CNTATCATQRLANFLVHSSNNFGPILPPKNVGSNTY

SEQ ID NO: 113 - protein, 36 aa, Artificial sequence; identical to SEQ ID NO: 85
  Synthetic polypeptide

SEQ ID NO: 114 - protein, 36 aa, Artificial sequence
  Synthetic polypeptide
CNTATCATQRLANFLKHSSNNFGPILPPTNVGSNTY

SEQ ID NO: 115 - protein, 36 aa, Artificial sequence
  Synthetic polypeptide
CNTATCATQRLANFLVKSSNNFGPILPPTNVGSNTY

SEQ ID NO: 116 - protein, 36 aa, Artificial sequence
  Synthetic polypeptide
CNTATCATQRLANFLVHSKNNFGPILPPTNVGSNTY

SEQ ID NO: 117 - protein, 36 aa, Artificial sequence
  Synthetic polypeptide
CNTATCATQRLANFLVHSSNNFGPILPPTNKGSNTY

SEQ ID NO: 118 - protein, 36 aa, Artificial sequence
  Synthetic polypeptide
CNTATCATQRLANFLVHSSNNFGPILPPTNVKSNTY

SEQ ID NO: 119 - protein, 36 aa, Artificial sequence
  Synthetic polypeptide
CNTATCATQRLANFLVHSSNNFGPILPPTNVGKNTY

SEQ ID NO: 120 - protein, 36 aa, Artificial sequence
  Synthetic polypeptide
CNTATCATQRLANFLVHSSNNFGPILPPTNVGSKTY

SEQ ID NO: 121 - protein, 36 aa, Artificial sequence
  Synthetic polypeptide
CNTATCATQRLANFLVHSSNNFGPILPPTNVGSNKY

SEQ ID NO: 122 - protein, 63 aa, Artificial sequence
  Synthetic polypeptide
HGEGTFTSDLSKQMEEEAVRLFIEWLKNGGGKCNTATCVLGRLSQELHRLQTYPRTNTGSNTY

SEQ ID NO: 123 - protein, 74 aa, Artificial sequence
  Synthetic polypeptide
HGEGTFTSDLSKQLEEEAVRLFIEWLKNGGPSSGAPPPSGGGKCNTATCVLGRLSQELHRLQTYPRTNTGSNTY

SEQ ID NO: 124 - protein, 63 aa, Artificial sequence
  Synthetic polypeptide
HGEGTFTSDLSKQMEEEAVRLFIEWLKNGCGKCNTATCVLGRLSQELHRLQTYPRTNTGSNTY

(19) United States
(12) Patent Application Publication
(10) Pub. No.: US 2014/0221282 A1
(43) Pub. Date: Aug. 7, 2014

(54) LONG DURATION DUAL HORMONE CONJUGATES

(71) Applicants: ASTRAZENECA PHARMACEUTICALS, LP, WILMINGTON, DE (US); AMYLIN PHARMACEUTICALS, LLC, SAN DIEGO, CA (US)

(72) Inventors: Chengzao Sun, San Diego, CA (US); Behrouz Bruce Forood, San Diego, CA (US)

(73) Assignees: ASTRAZENECA PHARMACEUTICALS LP; AMYLIN PHARMACEUTICALS, LLC

(21) Appl. No.: 14/119,581

(22) PCT Filed: May 24, 2012
(86) PCT No.: PCT/US12/39440

§ 371 Date: Apr. 14, 2014

Related U.S. Application Data
(60) Provisional application No. 61/489,951, filed on May 25, 2011.

(51) Int. Cl. A61K 47/48; A61K 38/22; A61K 38/26

(52) U.S. Cl. CPC A61K 47/48215; A61K 38/26; A61K 38/22
USPC 514/5.3; 530/308; 514/11.7; 514/7.2

(57) ABSTRACT

There are provided compounds having two peptide hormones bound to a central water-soluble polymeric spacer and methods of use thereof.

### CROSS-REFERENCES TO RELATED APPLICATIONS

This application claims the benefit of U.S. Provisional Application No. 61/489,951, filed May 25, 2011, which is incorporated herein by reference in its entirety and for all purposes.

### REFERENCE TO A “SEQUENCE LISTING,” A TABLE, OR A COMPUTER PROGRAM LISTING APPENDIX SUBMITTED AS AN ASCII TEXT FILE

The Sequence Listing written in file 92494-838063_ST25.TXT, created May 24, 2012, 74,177 bytes, machine format IBM-PC, MS-Windows operating system, is hereby incorporated by reference.

### BACKGROUND OF THE INVENTION

The present application relates to compounds comprising a plurality of peptide hormones in combination with a water-soluble polymeric spacer. Specifically, there are provided long-duration dual hormone conjugates (LDDHCs) having good duration of action and high potency, improved safety profile (e.g., low immunogenicity, low kidney vacuole formation) and/or convenient dosing regimens including weekly, twice monthly or monthly administration and oral administration, and methods of use thereof.

Without wishing to be bound by any theory, it is believed that the combination of two or more peptides into a compound, either through covalent or non-covalent attachment and optionally through appropriate spacers and/or linkers, is useful for treating a variety of diseases and disorders. Exemplary compounds and methods of combining peptides are described in PCT Published Appl. No. WO 2007/022123, filed Aug. 11, 2006, and WO 2005/077072, filed Feb. 11, 2005, each of which is incorporated herein by reference in its entirety and for all purposes. Exemplary indications which can be treated by such combinations of peptides include diabetes of types I and II, gestational diabetes, hypoglycemia, obesity, overweight, Paget's disease, osteoporosis and disorders of the skeletal tissues, heart disease, renal failure, acute and chronic coronary artery ischemia, cardiac arrhythmia, peripheral vascular disease, hypertension, pulmonary hypertension, preeclamptic toxemia, dyslipidemia, insulin resistance, cellular apoptosis, atherosclerosis, congestive heart failure, stroke, gallbladder disease, osteoarthritis, sleep apnea, polycystic ovarian syndrome, cancers of the breast, prostate and colon, complications incident to general anesthesia, infections, varicose veins, acanthosis nigricans, eczema, exercise intolerance, hypercholesterolemia, cholelithiasis, thromboembolic disease and Syndrome X.

Each patent, patent application, and publication cited herein is hereby incorporated herein by reference in its entirety and for all purposes.

### BRIEF SUMMARY OF THE INVENTION

There are provided long duration compounds (i.e., “long duration dual hormone conjugates,” “LDDHCs”) which include at least two peptides each having a hormonal activity bound to a water-soluble polymeric spacer, optionally through linkers. Each peptide hormone has a biological activity which can be measured by a suitable biological assay. When conjugated in a LDDHC described herein, each peptide hormone thereof maintains the biological activity, although not necessarily the potency, which can be measured by a suitable biological assay in the absence of conjugation.

In a first aspect, there is provided a long-duration dual hormone conjugate (LDDHC) compound having the structure of Formula I

P₁-L₁-PS-L₂-P₂ (I)

wherein P₁ is a peptide hormone having a first biological activity; P₂ is a peptide hormone having a second biological activity; L₁ and L₂ are independently a bond or a linker; and PS is a water-soluble polymeric spacer having a molecular weight in the range 30-80 kDa; wherein the compound exhibits the first biological activity in a biological assay, and the compound exhibits the second biological activity in a biological assay.

In another aspect, there is provided a pharmaceutical composition which includes a LDDHC described herein in combination with a pharmaceutically acceptable excipient.

In yet another aspect, there is provided a method for treating a disease or disorder in a subject. The method includes administering a LDDHC described herein to a subject in need thereof in an amount effective to treat the disease or disorder. Also included is the use of a LDDHC in the manufacture of a medicament for therapeutic use as described herein.

In another aspect, there is provided a long-duration dual hormone conjugate (LDDHC) compound having the structure of Formula I

P₁-L₁-PS-L₂-P₂ (I)

wherein P₁ has a first biological activity and is an exendin, exendin analog or derivative thereof; P₂ has a second biological activity and is an amylin, amylin analog or derivative thereof; L₁ and L₂ are independently a bond or a linker; and PS is a water-soluble polymeric spacer having a molecular weight in the range 30-80 kDa; wherein the compound exhibits the first biological activity in a biological assay, and the compound exhibits the second biological activity in a biological assay.

In each of the embodiments described above and herein Cmpd 14 is a most preferred compound or polypeptide conjugate.

### BRIEF DESCRIPTION OF THE DRAWINGS

FIGS. 1A-1B. FIG. 1A depicts schematic representations of LDDHC compounds as described herein, showing the topological relationship of P₁, P₂, PS and optional linkers L₁ and L₂. The term “PS” in FIGS. 1A-B is understood to representative all suitable water-soluble polymeric spacers contemplated for the compounds described herein. FIG. 1B depicts schematic representations of the formation of “T” linked and “C-terminal” or “N-terminal” compounds with a water-soluble polymeric spacer, as described below. In both FIGS. 1A-1B, a dashed line represents a linker, if present, or a bond attached to a side chain of either of peptides P₁ or P₂. It is understood that bonding between the backbone of either of P₁ or P₂ with PS can optionally include a linker.

FIGS. 2A-2C. FIG. 2A depicts SDS PAGE electrophoresis results for fractions from the FPLC purification of Cmpd 14 with 15 ml Macrocap™ SP column, as described in the Example section. FIG. 2B depicts the Maldi-TOF mass spectrum of Cmpd 14. Axes: x-axis: 19999.0 to 80002.0 (m/z); y-axis: % intensity. FIG. 2C depicts an SDS PAGE gel (NuPAGE® 4-12% Bis-Tris Gel) profile of Cmpd 14 at 3 μg and 10 μg.

FIGS. 3A-3B. FIG. 3A depicts the percent change in blood glucose with time following administration of compounds as described herein. Legend: vehicle (solid box); Cmpd 1 (exendin-4) (solid diamond); Cmpd 14 at 3 nmol/kg (solid triangle), 8 nmol/kg (open box), 25 nmol/kg (open triangle, tip up), 80 nmol/kg (open triangle, tip down), and 250 nmol/kg (open diamond); “*” p<0.05 vs. vehicle control (ANOVA, Dunnett's test). FIG. 3B depicts the percent change daily body weight (relative to pre-treatment weight) of the subjects of FIG. 3A. Legend: as for FIG. 3A.

FIGS. 4A-4B. FIG. 4A depicts the percent change in blood glucose with time following administration of compounds described herein. Compounds depicted were administered at 250 nmol/kg. Legend: vehicle (solid box); Cmpd 9 (“+”); Cmpd 8 (open diamond); “*” p<0.05 vs. vehicle control (ANOVA, Dunnett's test). FIG. 4B depicts the percent change in daily body weight (relative to pre-treatment weight) of the subjects of FIG. 4A. Legend: as for FIG. 4A.

FIGS. 5A-5B. FIG. 5A depicts the percent change in blood glucose with time following administration of compounds described herein. Legend: Cmpd 11 at 25 nmol/kg (“+”); Cmpd 11 at 80 nmol/kg (diamond); Cmpd 14 at 25 nmol/kg (triangle tip up); Cmpd 14 at 80 nmol/kg (triangle tip down); “*” p<0.05 vs. vehicle control (ANOVA, Dunnett's test). Points represent mean±SD (standard deviation). Peptide was injected SC (subcutaneous) at t=0 immediately following baseline sample into NIH/Swiss female mice. Samples were taken at t=2, 4, and 8 hours during day-1 and then daily through day-5. Blood glucose was measured with a OneTouch® Ultra® (LifeScan, Inc., Milpitas, Calif.). FIG. 5B depicts the percent change in daily body weight (relative to pre-treatment weight) of the subjects of FIG. 5A. Legend: as for FIG. 5A. Points represent mean±SD. Peptide was injected SC (subcutaneous) at t=0 immediately following baseline sample into NIH/Swiss female mice. Samples were taken daily through day-5.

FIGS. 6A-6B. FIG. 6A depicts the percent change in blood glucose with time following administration of compounds described herein. All compounds were administered at 25 nmol/kg with the exception of Cmpd 1 at 2.5 nmol/kg. Points represent mean±SD. Peptide was injected SC (subcutaneous) at t=0 immediately following baseline sample into NIH/Swiss female mice. Samples were taken at t=2, 4, and 8 hours during day-1 and then daily through day-5. Legend: Cmpd 1 (diamond); Cmpd 5 (“+”); Cmpd 7 (box); Cmpd 14 (triangle); “*” p<0.05 vs. vehicle control (ANOVA, Dunnett's test). FIG. 6B depicts the daily percent change in body weight (relative to pre-treatment weight) of the subjects of FIG. 6A. Legend: as for FIG. 6A. Points represent mean±SD. Peptide was injected SC (subcutaneous) at t=0 immediately following baseline sample into NIH/Swiss female mice. Samples were taken daily through day-5.

FIGS. 7A-7B. FIG. 7A depicts cumulative food intake in diet induced obese (DIO) rats after administration of compounds described herein. Dosing of all compounds (32 nmol/kg) was by SC injection on day 0 and day 7. Legend: Vehicle (box); Cmpd 7 (triangle tip up); Cmpd 6 (triangle tip down); combination of Cmpd 6 and Cmpd 7 (diamond); Cmpd 14 (circle). FIG. 7B depicts the percent daily body weight change (vehicle corrected) of the subjects of FIG. 7A. Legend: as for FIG. 7A. The arrows in FIG. 7B indicate the time of injection.

FIG. 8. FIG. 8 depicts the time course of the change in raw body weight for the assay depicted in FIGS. 7A-7B. Legend: as for FIG. 7B. See Example 8.

FIGS. 9A-9B. FIG. 9A depicts the time course of the change in body weight (% vehicle corrected) for Cmpd 14 at the indicated doses in a pharmacokinetic study. Legend: vehicle (box); Cmpd 14 at 0.5 mg/kg (triangle tip up); Cmpd 14 at 1.5 mg/kg (triangle tip down); Cmpd 14 at 3.0 mg/kg (diamond); arrows indicate blood collection. FIG. 9B depicts a histogram of the pharmacodynamic (plasma data) corresponding to the data provided in FIG. 9A. For each histogram group, the concentration of Cmpd 14 (“[Cmpd 14]”) goes in the order 0.5, 1.5 and 3.0 mg/kg. The dashed line indicates the lower limit of quantification (LLOQ). The numbers above the bars at days 15 and 21 indicate the number of positive samples of 6.

FIGS. 10A-10B. FIG. 10A depicts the time course for cumulative food intake (per cage, percent vehicle corrected) for the indicated compounds. All compounds were administered SC on days 0-6. Legend: vehicle (closed circle); Cmpd 6 at 10.95 mg/kg (box); Cmpd 7 at 11.1 mg/kg (“+”); Cmpd 14 at 1.2 mg/kg (triangle tip down); Cmpd 14 at 3.9 mg/kg (triangle tip up); Cmpd 14 at 12.3 mg/kg (diamond). FIG. 10B depicts the percent change in body weight (vehicle corrected) for the test data described in FIG. 10A. Legend: as for FIG. 10A. See Example 10.

FIG. 11. Change in body weight over time upon single injection of test compound. See Example 13. Legend: vehicle (closed box); Cmpd 45 (triangle tip up); Cmpd 46 (triangle tip down); Cmpd 47 (diamond); Cmpd 14a (closed circle); Cmpd 14 (open box). See Example 11.

FIGS. 12A-12B. FIG. 12A depicts percent change in body weight (vehicle corrected) over time upon single injection of test compound. See Example 14. Legend: Vehicle (closed box); Cmpd 48 (0.5 mg/kg) (triangle tip up); Cmpd 48 (1.5 mg/kg) (triangle tip down); Cmpd 48 (3.0 mg/kg) (diamond); Cmpd 14 (1.5 mg/kg) (circle). FIG. 12B depicts histogram of plasma drug levels at 3, 7, 14 and 20 days. For each histogram group, the compounds are presented in order (left to right): Cmpd 48 (0.5 mg/kg); Cmpd 48 (1.5 mg/kg); Cmpd 48 (3.0 mg/kg); and Cmpd 14 (1.5 mg/kg). The dashed line indicates the lower limit of quantification (LLOQ). The numbers above the bars at days 14 and 20 indicate the number of positive samples of 6. See Example 14.

FIGS. 13A-13B. FIG. 13A depicts the daily body weight percent change (vehicle corrected) results as described herein for Cmpds 69, 73, 72, 70, 74 and vehicle. FIG. 13B depicts the daily cumulative food intake results for Cmpds 69, 73, 72, 70, 74 and vehicle. Legend (FIGS. 13A-13B): Cmpd 69 (box); Cmpd 73 (triangle tip up); Cmpd 72 (triangle tip down); Cmpd 70 (diamond); Cmpd 74 (open circle); vehicle (filled circle). See Example 17.

FIGS. 14A-14B. FIG. 14A depicts baseline body weight (vehicle corrected) of comparison of twice weekly SC dosing of Cmpd 74 and continuous dosing of Cmpd 49 for two weeks in DIO rats. Legend: Vehicle (filled circle); Cmpd 74 (triangle); Cmpd 49 (box). FIG. 14B depicts the percent change in baseline body weight (vehicle corrected) of comparison of once weekly SC dosing of Cmpd 71 and continuous infusion of Cmpd 49 for four weeks in DIO rats. Legend: Vehicle (filled circle); Cmpd 71 (triangle); Cmpd 49 (box). See Example 18.

FIGS. 15A-15B. FIG. 15A depicts the daily cumulative body weight gain results (percent change from baseline body weight, vehicle corrected) from a dose response study for Cmpd 71. FIG. 15B depicts the cumulative daily food intake results from the dose response study for Cmpd 71. Legend (FIGS. 15A-15B): vehicle (box); 12 nmol/kg (triangle tip up); 25 nmol/kg (triangle tip down); 50 nmol/kg (diamond); 125 nmol/kg (filled circle); 250 nmol/kg (open box). See Example 18.

FIGS. 16A-16B. FIG. 16A depicts the cumulative percent change in body weight (vehicle corrected) from a dose response study for Cmpd 67. Legend: vehicle (box); Cmpd 67 at 80 nmol/kg (triangle tip up); Cmpd 67 at 160 nmol/kg (triangle tip down); Cmpd 67 at 320 nmol/kg (diamond). FIG. 16B depicts the percent change in daily body weight (vehicle corrected) as described herein for Cmpds 71, 75 and vehicle. Legend: vehicle (dark box); Cmpd 71 (light box); Cmpd 75 (triangle). See Example 19.

FIGS. 17A-17B. FIG. 17A depicts the daily percent change in body weight as described herein for Cmpds 74, 76, 77, 78 and vehicle. FIG. 17B depicts the daily food intake results for Cmpds 74, 76, 77, 78 and vehicle. Legend (FIGS. 17A-17B): Cmpd 74 (triangle tip down); Cmpd 76 (diamond); Cmpd 77 (large filled circle); Cmpd 78 (open box); vehicle (small filled circle). See Example 20.

FIGS. 18A-18B. FIG. 18A depicts the daily percent body weight change (vehicle corrected) results for a dose response study for Cmpd 77, and in comparison to Cmpd 79. FIG. 18B depicts the daily food intake results for Cmpd 77, and in comparison to Cmpd 79. Legend (FIGS. 18A-18B): Cmpd 79, 250 nmol/kg (box); Cmpd 77, 250 nmol/kg (triangle tip up); Cmpd 77, 125 nmol/kg (triangle tip down); Cmpd 77, 62.5 nmol/kg (diamond); Cmpd 77, 31.25 nmol/kg (large filled circle); vehicle (small filled circle). See Example 20.

FIGS. 19A-19B. FIG. 19A depicts the daily percent body weight change (vehicle corrected) in lean rats after a single SC injection at 125 nmol/kg of the test compound as determined over 7-days. FIG. 19B depicts the corresponding cumulative food intake percent change (vehicle corrected) during the test period. Legend: Vehicle (closed circle); Cmpd 77 (closed box); Cmpd 80 (diamond); Cmpd 81 (open circle); Cmpd 82 (open box); Cmpd 83 (triangle tip up); Cmpd 84 (triangle tip down). See Example 21.

### DETAILED DESCRIPTION OF THE INVENTION

### I. Definitions

The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched chain, or combination thereof, which may be fully saturated, mono- or polyunsaturated and can include di- and multivalent radicals, having the number of carbon atoms designated (i.e., C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, (cyclohexyl)methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—).

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene.

The term “heteroalkyl,” by itself or in combination with another term, means, unless otherwise stated, a stable straight or branched chain, or combinations thereof, consisting of at least one carbon atom and at least one heteroatom selected from the group consisting of O, N, P, Si, and S, and wherein the nitrogen and sulfur atoms may optionally be oxidized, and the nitrogen heteroatom may optionally be quaternized. The heteroatom(s) O, N, P, S, and Si may be placed at any interior position of the heteroalkyl group or at the position at which the alkyl group is attached to the remainder of the molecule. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, —O—CH₂—CH₃, and —CN. Up to two heteroatoms may be consecutive, such as, for example, —CH₂—NH—OCH₃.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The terms “cycloalkyl” and “heterocycloalkyl,” by themselves or in combination with other terms, mean, unless otherwise stated, cyclic versions of “alkyl” and “heteroalkyl,” respectively. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring. The term “heteroaryl” refers to aryl groups (or rings) that contain from one to four heteroatoms selected from N, O, and S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively.

For brevity, the term “aryl” when used in combination with other terms (e.g., aryloxy, arylthioxy, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl, and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like).

The term “oxo,” as used herein, means an oxygen that is double bonded to a carbon atom.

The term “alkylsulfonyl,” as used herein, means a moiety having the formula —S(O₂)—R′, where R′ is an alkyl group as defined above. R′ may have a specified number of carbons (e.g., “C₁-C₄ alkylsulfonyl”).

Each of the above terms (e.g., “alkyl,” “heteroalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN, and —NO₂ in a number ranging from zero to (2m′+1), where m′ is the total number of carbon atoms in such radical. R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted alkyl, alkoxy, or thioalkoxy groups, or arylalkyl groups. When a compound described herein includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN, —NO₂, —R′, —N₃, —CH(Ph)₂, fluoro(Cⱼ—C₄)alkoxy, and fluoro(C₁-C₄)alkyl, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound described herein includes more than one R group, for example, each of the R groups is independently selected as are each R′, R″, R′″, and R″″ groups when more than one of these groups is present.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}-U-, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′—(C″R′″)_{d}—, where s and d are independently integers of from 0 to 3, and X′ is —O—, —NR′—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include oxygen (O), nitrogen (N), sulfur (S), phosphorus (P), and silicon (Si).

A “substituent group,” as used herein, means a group selected from the following moieties:
(A) —OH, —NH₂, —SH, —CN, —CF₃, —NO₂, oxo, halogen, unsubstituted alkyl, unsubstituted heteroalkyl, unsubstituted cycloalkyl, unsubstituted heterocycloalkyl, unsubstituted aryl, unsubstituted heteroaryl, and
(B) alkyl, heteroalkyl, cycloalkyl, heterocycloalkyl, aryl, and heteroaryl, substituted with at least one substituent selected from:
   (i) oxo, —OH, —NH₂, —SH, —CN, —CF₃, —NO₂, halogen, unsubstituted alkyl, unsubstituted heteroalkyl, unsubstituted cycloalkyl, unsubstituted heterocycloalkyl, unsubstituted aryl, unsubstituted heteroaryl, and
   (ii) alkyl, heteroalkyl, cycloalkyl, heterocycloalkyl, aryl, and heteroaryl, substituted with at least one substituent selected from:
      (a) oxo, —OH, —NH₂, —SH, —CN, —CF₃, —NO₂, halogen, unsubstituted alkyl, unsubstituted heteroalkyl, unsubstituted cycloalkyl, unsubstituted heterocycloalkyl, unsubstituted aryl, unsubstituted heteroaryl, and
      (b) alkyl, heteroalkyl, cycloalkyl, heterocycloalkyl, aryl, or heteroaryl, substituted with at least one substituent selected from: oxo, —OH, —NH₂, —SH, —CN, —CF₃, —NO₂, halogen, unsubstituted alkyl, unsubstituted heteroalkyl, unsubstituted cycloalkyl, unsubstituted heterocycloalkyl, unsubstituted aryl, and unsubstituted heteroaryl.

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₄-C₈ cycloalkyl, and each substituted or unsubstituted heterocycloalkyl is a substituted or unsubstituted 4 to 8 membered heterocycloalkyl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₅-C₇ cycloalkyl, and each substituted or unsubstituted heterocycloalkyl is a substituted or unsubstituted 5 to 7 membered heterocycloalkyl.

The term “pharmaceutically acceptable salts” is meant to include salts of the active compounds that are prepared with relatively nontoxic acids or bases, depending on the particular substituents found on the compounds described herein. When compounds described herein contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds described herein contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogen carbonic, phosphoric, monohydrogen phosphoric, dihydrogen phosphoric, sulfuric, monohydrogen sulfuric, hydro iodic, or phosphorous acids and the like, as well as the salts derived from relatively nontoxic organic acids like acetic, propionic, is butyric, maleic, masonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds described herein contain both basic and acidic functionalities that allow the compounds to be converted into either base or acid addition salts.

Thus, the compounds described herein may exist as salts, such as with pharmaceutically acceptable acids. In one embodiment, the compounds and compositions described herein includes such salts. Examples of such salts include hydrochlorides, hydrobromides, sulfates, methanesulfonates, nitrates, maleates, acetates, citrates, fumarates, tartrates (e.g., (+)-tartrates, (−)-tartrates, trifluoroacetates, or mixtures thereof including racemic mixtures), succinates, benzoates, and salts with amino acids such as glutamic acid. These salts may be prepared by methods known to those skilled in the art.

The neutral forms of the compounds are preferably regenerated by contacting the salt with a base or acid and isolating the parent compound in the conventional manner. The parent form of the compound differs from the various salt forms in certain physical properties, such as solubility in polar solvents.

In one embodiment the salts are acetate, hydrochloride or trifluoroacetate.

Certain compounds described herein can exist in unsolvated forms as well as solvated forms, including hydrated forms. In general, the solvated forms are equivalent to unsolvated forms and are encompassed within the scope described herein. Certain compounds described herein may exist in multiple crystalline or amorphous forms. In general, all physical forms are equivalent for the uses contemplated herein and are intended to be within the scope described herein.

Certain compounds described herein possess asymmetric carbon atoms (optical centers) or double bonds; and the racemates, diastereomers, tautomers, geometric isomers, and individual isomers are encompassed within the scope described herein. The compounds described herein do not include those compounds known in the art to be too unstable to synthesize and/or isolate.

The compounds described herein may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds described herein, whether radioactive or not, are encompassed within the scope described herein.

The symbol “ [symbol] ” denotes the point of attachment of a chemical moiety to the remainder of a molecule or chemical formula.

“Ortholog” and like terms in the context of peptides refer to two or more peptide gene products wherein the genes coding the ortholog have evolved from a common ancestor, as known in the art.

“Analog” as used herein in the context of polypeptides refers to a compound that has insertions, deletions, additions and/or substitutions of amino acids relative to a parent compound. An analog may have superior stability, solubility, efficacy, half-life, and the like. In one embodiment, an analog is a compound having at least 45%, for example 45%, 50%, 55%, 60%, 65%, 70%, 75%, 80%, 85%, 90%, 95%, 98%, or even higher, sequence identity to the parent compound. In one embodiment the analog has no more than 20, 19, 20, 17, 16, 15, 10, 6, 5, 4, 3, 2, and/or 1 insertions, deletions, additions and/or substitutions relative to the parent compound. Exemplary parent compounds include exendin-4, GLP-1, rat amylin, pramlintide, davalintide, and the other parent compounds described herein. The addition may be an extension such as the exendin-4 “tail” or frog GLP-1 “tail” or fragment or analog thereof, as disclosed in PCT Published Appl. Nos. WO 2007/022123 and WO 2005/077072. Exemplary extensions include KNGGPSSGAPPPS (SEQ ID NO:1), PSSGAPPPS(SEQ ID NO:2). FIEWLKNGGPSSGAPPPS(SEQ ID NO:3) and PKKIRYS(SEQ ID NO:4) and analogs thereof.

The term “derivative” in the context of a polypeptide refers to a molecule having the amino acid sequence of a parent or analog thereof, but additionally having a chemical modification of one or more of its amino acid side groups, α-carbon atoms, terminal amino group, or terminal carboxylic acid group. A chemical modification includes, but is not limited to, adding chemical moieties, creating new bonds, and removing chemical moieties. Modifications at amino acid side groups include, without limitation, acylation of lysine E-amino groups, N-alkylation of arginine, histidine, or lysine, alkylation of glutamic or aspartic carboxylic acid groups, and deamidation of glutamine or asparagine. Modifications of the terminal amino include, without limitation, the desamino, N-lower alkyl, N-di-lower alkyl, constrained alkyls (e.g. branched, cyclic, fused, adamantyl) and N-acyl modifications. Modifications of the terminal carboxy group include, without limitation, the amide, lower alkyl amide, constrained alkyls (e.g. branched, cyclic, fused, adamantyl) alkyl, dialkyl amide, and lower alkyl ester modifications. Furthermore, one or more side groups, or terminal groups, may be protected by protective groups known to the ordinarily-skilled synthetic chemist. The alpha-carbon of an amino acid may be mono- or dimethylated.

Derivatives also contemplate polypeptides in which the stereochemistry of individual amino acids is inverted (i.e., (L)/S to (D)/R) at one or more specific sites. Also contemplated are polypeptides modified by glycosylation (e.g., at Asn, Ser and/or Thr residues). Polypeptide components useful in the compounds and methods described herein may also be biologically active fragments of the parent peptides (native, agonist, analog, and derivative) described herein.

The terms “identity,” “sequence identity” and the like in the context of comparing two or more nucleic acids or polypeptide sequences, refer to two or more sequences or subsequences that are the same or have a specified percentage of amino acid residues or nucleotides that are the same (i.e., about 50% identity, preferably 50%, 55%, 60%, 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a sequence comparison algorithms as known in the art, for example BLAST or BLAST 2.0. This definition includes sequences that have deletions and/or additions, as well as those that have substitutions, as well as naturally occurring, e.g., polymorphic or allelic variants, and man-made variants. In preferred algorithms, account is made for gaps and the like, as known in the art. For sequence comparison, typically one sequence acts as a reference sequence, to which test sequences are compared. When using a sequence comparison algorithm, test and reference sequences are entered into a computer, subsequence coordinates are designated if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters. Optimal alignment of sequences for comparison can be conducted, e.g., by the local homology algorithm of Smith & Waterman, 1981, Adv. Appl. Math. 2:482, by the homology alignment algorithm of Needleman & Wunsch, 1970, J. Mol. Biol. 48:443, by the search for similarity method of Pearson & Lipman, 1988, Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection. See e.g., Current Protocols in Molecular Biology (Ausubel et al., eds. 1995 supplement)). Preferred examples of algorithms that are suitable for determining percent sequence identity and sequence similarity include the BLAST and BLAST 2.0 algorithms, which are described in Altschul et al., 1977, Nuci. Acids Res. 25:3389-3402 and Altschul et al., 1990, J. Mol. Biol. 215:403-410. BLAST and BLAST 2.0 are used, as known in the art, to determine percent sequence identity for the nucleic acids and proteins or peptide described herein. Software for performing BLAST analyses is publicly available through the web site of the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., Id.). These initial neighborhood word hits act as seeds for initiating searches to find longer HSPs containing them. The word hits are extended in both directions along each sequence for as far as the cumulative alignment score can be increased. Cumulative scores are calculated using, e.g., for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, 1989, Proc. Nati. Acad. Sci. USA 89:10915) alignments (B) of 50, expectation (E) of 10, M=5, N=−4, and a comparison of both strands.

To determine the percent identity or similarity of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same or similar amino acid residue or nucleotide as the corresponding position in the second sequence, then the molecules are identical or similar at that position. The percent identity or similarity between the two sequences is a function of the number of identical or similar positions shared by the sequences (i.e., % identity=# of identical positions/total # of positions (e.g., overlapping positions)×100). The similarity of two amino acids can be assessed by a variety of methods known in the art. For example, nonpolar neutral residues (e.g., Ala, Cys, Gly, Ile, Leu, Met, Phe, Pro, Trp, Val) can be considered similar, as can in turn acidic charged polar (e.g., Glu, Asp), basic charged polar (e.g., Arg, His, Lys) and neutral polar (e.g., Asn, Gln, Ser, Thr, Tyr) residues.

Both identity and similarity may be readily calculated. For example, in calculating percent identity, only exact matches may be counted, and global alignments may be performed as opposed to local alignments. Methods commonly employed to determine identity or similarity between sequences include, e.g., those disclosed in Carillo et al., 1988, SIAM J. Applied Math. 48:1073. Exemplary methods to determine identity are designed to give the largest match between the sequences tested. Exemplary methods to determine identity and similarity are also provided in commercial computer programs. A particular example of a mathematical algorithm utilized for the comparison of two sequences is the algorithm of Karlin et al., 1990, Proc. Natl. Acad. Sci. USA 87:2264-2268, and as modified e.g., as in Karlin et al., 1993, Proc. Natl. Acad. Sci. USA 90:5873-5877. Such an algorithm is incorporated into the NBLAST and XBLAST programs of Altschul et al., 1990, J. Mol. Biol. 215:403-410. To obtain gapped alignments for comparison purposes, Gapped BLAST can be utilized as described in Altschul et al., 1997, Nucleic Acids Res. 25:3389-3402. Alternatively, PSI-Blast can be used to perform an iterated search, which detects distant relationships between molecules. When utilizing BLAST, Gapped BLAST, and PSI-Blast programs, the default parameters of the respective programs (e.g., XBLAST and NBLAST) can be used, as known in the art. Additionally, the FASTA method (Atschul et al., 1990, Id.) can be used. Another particular example of a mathematical algorithm useful for the comparison of sequences is the algorithm of Myers et al., 1988, CABIOS 4: 11-17. Such an algorithm is incorporated into the ALIGN program (version 2.0), which is part of the GCG sequence alignment software package (Devereux et al., 1984, Nucleic Acids Res. 12(1):387). Percent identity can be determined by analysis with the AlignX® module in Vector NTI® (Invitrogen; Carlsbad Calif.).

“Obesity” and “overweight” refer to mammals having a weight greater than normally expected, and may be determined by, e.g., physical appearance, body mass index (BMI) as known in the art, waist-to-hip circumference ratios, skinfold thickness, waist circumference, and the like. The Centers for Disease Control and Prevention (CDC) define overweight as an adult human having a BMI of 25 to 29.9; and define obese as an adult human having a BMI of 30 or higher. Additional metrics for the determination of obesity exist. For example, the CDC states that a person with a waist-to-hip ratio greater than 1.0 is overweight.

“Lean body mass” refers to the fat-free mass of the body, i.e., total body weight minus body fat weight is lean body mass. Lean body mass can be measured by methods such as hydrostatic weighing, computerized chambers, dual-energy X-ray absorptiometry, skin calipers, magnetic resonance imaging (MRI) and bioelectric impedance analysis (BIA) as known in the art.

“Mammal” refers to warm-blooded animals that generally have fur or hair, that give live birth to their progeny, and that feed their progeny with milk. Mammals include humans; companion animals (e.g., dogs, cats); farm animals (e.g., cows, horses, sheep, pigs, goats); wild animals; and the like. In one embodiment, the mammal is a female. In one embodiment, the mammal is a female human. In one embodiment, the mammal is a cat or dog. In one embodiment, the mammal is a diabetic mammal, e.g., a human having type 2 or type 1 diabetes. In one embodiment, the mammal is an obese diabetic mammal, e.g., an obese human having type 2 or type 1 diabetes.

“Fragment” in the context of polypeptides refers herein in the customary chemical sense to a portion of a polypeptide. For example, a fragment can result from N-terminal deletion or C-terminal deletion of one or more residues of a parent polypeptide, and/or a fragment can result from internal deletion of one or more residues of a parent polypeptide. The term “parent” in the context of polypeptides refers, in the customary sense, to a polypeptide which serves as a reference structure prior to modification, e.g., insertion, deletion and/or substitution. The term “conjugate” in the context of conjugated polypeptides described herein refers to the formation of covalent linkage between component polypeptides, linkers and water-soluble polymeric spacers.

The terms “long-duration dual-hormone conjugate,” “LDDHC” and the like refer to compounds having the structure of Formula (I) as described herein.

The terms “peptide” and “polypeptide” in the context of the LDDHCs described herein are synonymous. The term “peptide” refers in the customary sense to a polymer of amino acids connected by amide bonds. The terms “des-amino acid,” “des-AA,” “desLys,” “des-Lys” and the like refer to the absence of the indicated amino acid, as customarily used in the art. An amino acid being “absent” means that the residues (or functionalities) formerly attached at the N-terminal and C-terminal side of the absent amino acid (or functionality) have become bonded together. The terms “peptide component” and “polypeptide component” refer to polypeptides included within a LDDHC described herein.

It should be noted that throughout the application that alternatives are written in Markush groups, for example, each amino acid position that contains more than one possible amino acid. It is specifically contemplated that each member of the Markush group should be considered separately, thereby comprising another embodiment, and the Markush group is not to be read as a single unit.

As used herein, the singular form “a”, “an”, and “the” includes plural references unless otherwise indicated or clear from context. For example, as will be apparent from context, “a” analog can include one or more analogs. Unless indicated otherwise, the term “about” in the context of a numeric value refers to +/−10% of the numeric value.

### II. Compounds

It is understood that the terms “compound” and the like in the context of compounds useful for a compound, composition or method described herein refer to long duration dual hormone conjugates (“LDDHCs”). Applicants have made the surprising observation of the problem that certain peptides, when attached directly or optionally through a short linker, or through relatively short spacers (i.e., “short peptide conjugates”), may interfere with each other's biological activities, as judged, for example, by a reduction in potency of one or more of the peptides in a suitable assay. The term “short” in the context of the size of linkers and water-soluble polymeric spacers described herein refers to a size sufficiently small that interference is observed between the peptide hormones included within the short peptide conjugates. The Applicants have also made the surprising discovery of the problem that applying standard pegylation approaches, as known in the art, such as pegylation at an N-terminal or C-terminal amino acid, or at a side-chain residue of an internal or terminal amino acid, to such short hybrid peptide conjugates may additionally or further reduce the potency of one or more peptides contained within the short peptide conjugate. The term “pegylation” refers, as customary in the art, to covalent addition of polyethylene glycol.

The terms “having a first biological activity,” “having a second biological activity” and the like mean that the peptide hormone is capable of eliciting an in vitro biochemical response (e.g., receptor binding, cAMP cyclase activation and the like) and/or an in vivo physiologic (i.e., biological) response (e.g., change in blood glucose, change in body weight and the like). Accordingly, the term “activity” in these contexts refers to specific biochemical or biological responses which are characterized by specific biochemical and/or biological changes. The terms “potency,” “full potency,” “low potency,” “high potency” and the like refer to the capacity of a compound to elicit a response. For example, a highly potent compound elicits a larger response than a lower potency compound when the compounds are administered in equimolar concentrations. The terms “efficacy,” “full efficacy,” “low efficacy,” “high efficacy” and the like refer to the capacity of a compound to elicit a response relative to its maximal response or that of a comparator compound.

An exemplary short peptide conjugate wherein interference, as judged by reduced potency in a suitable assay, can be observed is pegylated exendin-4-beta alanine-beta alanine-amylin short peptide conjugates (e.g., C-terminal amino acid of exendin-4 linked via a beta-alanine-beta-alanine linker to the N-terminal amino acid of amylin). Accordingly, the present disclosure is based in part on the surprising discovery that physically separating such peptides by use of large, e.g., 30 to 80 kDa, water soluble polymer spacers provides a solution to the above described problems and results in surprisingly superior compounds (i.e., LDDHCs). The LDDHCs described herein have surprisingly superior properties including an increased duration of action, high potency, high efficacy, improved safety profile (e.g. low immunogenicity, low kidney vacuole formation) and/or more convenient dosing regimens including weekly, twice monthly or monthly administration and oral administration, and methods of therapeutic use thereof.

In a first aspect, there is provided a long-duration dual-hormone conjugate (LDDHC) having the structure of Formula I:

P₁-L₁-PS-L₂-P₂ (I).

In Formula I, P₁ is a peptide hormone having a first biological activity. P₂ is a peptide hormone having a second biological activity. L₁ and L₂ are independently a bond or a linker. PS is a peptidic or non-peptidic water-soluble polymeric spacer having a molecular weight in the range 30-80 kDa. In a preferred embodiment PS is non-peptidic. In a more preferred embodiment, PS is a polyethylene glycol of the defined size. A most preferred compound of Formula I is Cmpd 14.

In one embodiment, PS is of sufficient size to provide both a long duration of action and a physical or functional separation of the peptide hormones included in the LDDHC such that a biological activity of one or both peptide hormones is improved compared to a reference conjugate. The term “reference conjugate” refers to a peptide conjugate as described herein, which lacks the PS as a spacer, which includes a short PS spacer, or which lacks the PS as a spacer but has the PS attached at a pendant amino acid side chain of the reference conjugate backbone.

In one embodiment, PS has a size in the range of 30-80 kDa, preferably 35-50 kDa. P₁ and P₂ can be independently attached to L₁ or L₂ at an N-terminus, C-terminus or pendant side chain position along P₁ or P₂. Exemplary sites of attachment along P₁ or P₂ include, but are not limited to, the epsilon amino group of a lysine amino acid residue or the sulfhydryl group of a cysteine residue.

In one embodiment, the LDDHC exhibits both the first biological activity and also the second biological activity in suitable biological assays. In one embodiment, the first and second biological activities are the same. In one embodiment, the first and second biological activities are different. Exemplary biological activities include the biological activities of exendin, amylin, pramlintide or davalintide as described herein. In one embodiment, the LDDHC exhibits one of the first biological activity or the second biological activity in suitable biological assays.

In one embodiment, the LDDHC can be administered once daily, once every second or third day, once weekly, twice monthly or monthly, while retaining a desired clinical profile.

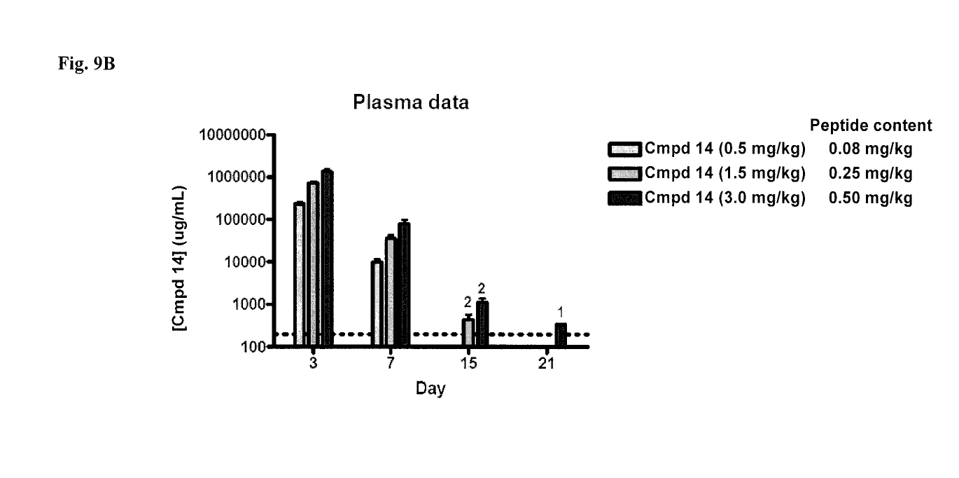

In another aspect, the LDDHC has a significantly improved half-life in a rodent model relative to either parent peptide or to a reference conjugate of the parent peptides, as judged by a suitable assay. Preferred is a half-life longer than 12 hours, preferably at least 1 day, 2 days, 3 days, 4 days or at least 5 days or longer in a mouse or rat model, with most preferred being at least 20 hours as determined in a rat, e.g. a t_{1/2} of a least 22 hours such as for Cmpd 14 in a rat (see FIG. 9, for example). Preferred is a half-life longer than 5 hours in pigs, monkeys or humans using intravenous (IV) or subcutaneous (SC) peripheral dosing, preferably longer than 12 hours, more preferably at least 1 day, 2 days, 3 days, 6 days, 7 days or even longer.

As known in the art and described herein, covalent bonding of peptide hormones to form a multi-peptide compound can result in reduction of potency of one or more of the peptide hormones thereof relative to the potency of the peptide hormones alone, as judged by suitable assays. “Multi-peptide compound” refers to a compound resulting from covalent bonding of a plurality of peptides, optionally through linkers as described herein and known in the art. Multi-peptide compounds can additionally include short water-soluble polymeric spacers, as described herein; e.g., short peptide conjugates. Indeed, such multi-peptide compounds can be devoid (i.e., have no potency) for one or more of the activities of the individual peptides forming the multi-peptide compounds.

In contrast, it has been surprisingly found that LDDHCs described herein can maintain the individual biological activities of the constituent peptides (i.e., P₁ and P₂ of Formula I) as assessed in suitable assays. It has been further found that the size of the water-soluble polymeric spacer (PS) can determine whether a particular biological activity (i.e., of P₁ and/or P₂) is also observed with the LDDHC.

Peptide hormone contemplated as elements of the LDDHCs described herein are understood to include the naturally occurring hormone, and analogs and derivatives thereof. Exemplary peptide hormones include those following described.

Exendins.

In certain embodiments, P₁ is an exendin, exendin analog or a derivative thereof. In one embodiment, P₁ is an exendin. In one embodiment, P₁ is an exendin analog. In one embodiment, P₁ is a derivative of exendin. Exendin, exendin analogs and derivatives thereof suitable for use in the LDDHCs and methods described herein include the compounds described in WO 2007/022123 (PCT/US2006/031724, filed Aug. 11, 2006), incorporated herein by reference and for all purposes. The exendins are peptides that are found in the salivary secretions of the Gila monster and the Mexican Bearded Lizard, reptiles that are endogenous to Arizona and Northern Mexico. Exendin-3 is present in the salivary secretions of Heloderma horridum (Mexican Beaded Lizard), and exendin-4 is present in the salivary secretions of Heloderm suspectum (Gila monster). See Eng et al, 1990, J. Biol. Chem., 265:20259-62; Eng et al, 1992, J. Biol. Chem., 267:7402-7405. The sequences of exendin-3 and exendin-4, respectively, follow:

(SEQ ID NO: 5)
HSDGTFTSDLSKQMEEEAVRLFIEWLKNGGPSSGAPPPS-NH₂;
(SEQ ID NO: 6)
HGEGTFTSDLSKQMEEEAVRLFIEWLKNGGPSSGAPPPS-NH₂.

Hargrove et al. (Regulatory Peptides, 2007, 141:113-119) reported an exendin-4 peptide analog that is a full-length C-terminally amidated exendin-4 peptide analog with a single nucleotide difference at position 14 compared to native exendin-4. The sequence of [Leu¹⁴]Exendin-4 is: HGEGTFTSDLSKQLEEEAVRLFIEWLKNGGPSSGAPPPS-NH₂ (SEQ ID NO:7). Another exendin-4 peptide analog is a chimera of the first 32 amino acids of exendin-4 having amino acid substitutions at positions 14 and 28 followed by a 5 amino acid sequence from the C-terminus of a non-mammalian (frog) GLP1. This compound has the sequence: HGEGTFTSDLSKQLEEEAVRLFIEWLKQGGPSKEIIS (SEQ ID NO:8). Also known in the art are C-terminally truncated, biologically active forms of exendin-4, such as exendin-4(1-28), exendin-4(1-29) and exendin-4(1-30) and amidated forms thereof. All of these exendin analogs are suitable as polypeptide components of the LDDHCs described herein. It is understood that in some embodiments a C-terminal amide, or other C-terminal capping moiety can be present in polypeptide components described herein.

The exendins have some sequence similarity to several members of the glucagon-like peptide family, with the highest homology, 53%, being to GLP-1 [7-36]NH₂ (Goke et al, 1993, J. Biol. Chem., 268:19650-55), having sequence HAEGTFTSDVSSYLEGQAAKEFIAWLVKGRG (SEQ ID NO:9), also sometimes referred to as “GLP-1”, which has an insulinotropic effect, stimulating insulin secretion from pancreatic beta-cells. GLP-1 has also been reported to inhibit glucagon secretion from pancreatic alpha-cells. See e.g., Orskov et al, 1993, Diabetes, 42:658-61; D'Alessio et al, 1996, J. Clin. Invest., 97:133-38. GLP-1 has been reported to inhibit gastric emptying (Willms B, et al., 1996, J. Clin. Endocrinol. Metab., 81:327-32; Wettergren A, et al., 1993, Dig. Dis. Sci. 38:665-73) and gastric acid secretion (Schjoldager B T, et al, 1989, Dig. Dis. Sci., 34:703-8; O'Halloran D J, et al., 1990, J. Endocrinol., 126:169-73; Wettergren A, et al., 1993, Dig. Dis. Sci., 38:665-73). Peptide GLP-1[7-37], which has an additional glycine residue at its carboxy terminus, is reported to stimulate insulin secretion in humans. See Orskov, et al., 1993, Diabetes, 42:658-61. Other reports relate to the inhibition of glucagon secretion (Creutzfeldt W O C, et al., 1996, Diabetes Care, 19:580-6), and a purported role in appetite control (Turton M D, et al., 1996, Nature, 379(6560):69-72). A transmembrane G-protein adenylate-cyclase-coupled receptor, said to be responsible at least in part for the insulinotropic effect of GLP-1, has reportedly been cloned from a beta-cell line (Thorens, 1992, Proc. Natl. Acad. Sci. USA 89:8641-45). GLP-1 has been the focus of significant investigation in recent years due to its reported action on the amplification of stimulated insulin production (Byrne, M. M., Goke, B., “Lessons from human studies with glucagon-like peptide-1: Potential of the gut hormone for clinical use”. In: Fehmann, H. C., Goke, B., 1997, Insulinotropic Gut Hormone Glucagon-Like Peptide 1. Basel, Switzerland: Karger, 1997:219-33).

Pharmacological studies have led to reports that exendin-4 can act at GLP-1 receptors in vitro on certain insulin-secreting cells, at dispersed acinar cells from guinea pig pancreas, and at parietal cells from stomach; the peptide is also reported to stimulate somatostatin release and inhibit gastrin release in isolated stomachs. See e.g., Goke, et al., 1993, J. Biol. Chem. 268:19650-55; Schepp, et al., 1994, Eur. J. Pharmacol. 69:183-91; Eissele, et al., 1994, Life Sci. 55:629-34. Exendin-3 and exendin-4 were reportedly found to stimulate cAMP production in, and amylase release from, pancreatic acinar cells (Malhotra, R. et al., 1993, Regulatory Peptides 41:149-56; Raufman, et al., 1992, J. Biol. Chem. 267:21432-37; Singh, et al., 1994, Regul. Pept. 53:47-59). Exendin-4 has a significantly longer duration of action than GLP-1. For example, in one experiment, glucose lowering by exendin-4 in diabetic mice was reported to persist for several hours, and, depending on dose, for up to 24 hours (Eng, J. 1996 Diabetes 45 (Suppl 2):152A (abstract 554)). Based on the insulinotropic activities of these molecules, the use of exendin-3 and exendin-4 for the treatment of diabetes mellitus and the prevention of hyperglycemia has been proposed (Eng, U.S. Pat. No. 5,424,286, incorporated herein by reference in its entirety and for all purposes).

The results of an investigation which showed that exendins are not the species homolog of mammalian GLP-1 was reported by Chen & Drucker who cloned the exendin gene from the Gila monster (J. Biol. Chem. 272:4108-15 (1997)). The observation that the Gila monster also has separate genes for proglucagons (from which GLP-1 is processed), that are more similar to mammalian proglucagon than exendin, indicated that exendins are not merely species homologs of GLP-1.

Methods for regulating gastrointestinal motility using exendin agonists are described in U.S. Pat. No. 6,858,576 (i.e., based on U.S. application Ser. No. 08/908,867 filed Aug. 8, 1997, which is a continuation-in-part of U.S. application Ser. No. 08/694,954 filed Aug. 8, 1996). Methods for reducing food intake using exendin agonists are described in U.S. Pat. No. 6,956,026 (i.e., based on U.S. application Ser. No. 09/003,869, filed Jan. 7, 1998, which claims the benefit of U.S. Application Nos. 60/034,905 filed Jan. 7, 1997, 60/055,404 filed Aug. 7, 1997, 60/065,442 filed Nov. 14, 1997, and 60/066,029 filed Nov. 14, 1997.

Novel exendin agonist compounds are described in WO 99/07404 (i.e., PCT/US98/16387 filed Aug. 6, 1998, which claims the benefit of U.S. patent application Ser. No. 60/055,404, filed Aug. 8, 1997). Other novel exendin agonists are described in WO 99/25727 (i.e., PCT/US98/24210, filed Nov. 13, 1998, which claims the benefit of U.S. Provisional Application No. 60/065,442 filed Nov. 14, 1997). Still other novel exendin agonists are described in WO 99/25728 (i.e., PCT/US98/24273, filed Nov. 13, 1998, which claims the benefit of U.S. Provisional Application No. 60/066,029 filed Nov. 14, 1997). Other recent advances in exendin related technology are described in WO 99/40788 (i.e., which claims priority to U.S. Application No. 60/075,122, filed Feb. 13, 1998); and in WO 00/41546, and WO 00/41548 (i.e., which claim priority to U.S. Application No. 60/116,380, filed Jan. 14, 1998). instability, toxicity, and reactivity. Id.

In certain embodiments, P₁ includes from 1 to 39 residues. In certain embodiments, P₁ includes from 1 to 28 residues. In certain embodiments, P₁ is exendin-4. In certain embodiments, P₁ is exendin-4(1-28). In certain embodiments, P₁ is exendin-4(1-29). In certain embodiments, P₁ is exendin-4(1-30). In certain embodiments, P₁ is exendin-4(1-31). In certain embodiments, P₁ is exendin-4(1-32). In certain embodiments, P₁ is an exendin-4 analog. In certain embodiments, P₁ is a derivative of exendin-4 or an exendin-4 analog.

Exendins and exendin analogs useful for the LDDHCs described herein include compounds described in U.S. Pat. No. 7,223,725 (incorporated herein by reference and for all purposes), and compounds of Formula (IV) following:

(IV)
Xaa₁-Xaa₂-Xaa₃-Gly₄-Xaa₅-Xaa₆-Xaa₇-Xaa₈-Xaa₉-
Xaa₁₀-Xaa₁₁-Xaa₁₂-Xaa₁₃-Xaa₁₄-Xaa₁₅-Xaa₁₆-Xaa₁₇-
Ala₁₈-Xaa₁₉-Xaa₂₀-Xaa₂₁-Xaa₂₂-Xaa₂₃-Xaa₂₄-Xaa₂₅-
Xaa₂₆-Xaa₂₇-Xaa₂₈

wherein Xaa₁ is absent, His, Arg or Tyr; Xaa₂ is absent, Ser, Gly, Ala, Thr, Val, Leu, Lys, or Aib; Xaa₃ is Ala, Asp or Glu; Xaa₅ is Ala or Thr; Xaa₆ is Ala, Phe, Tyr; Xaa₇ is Thr or Ser; Xaa₈ is Ala, Ser or Thr; Xaa₉ is Asp or Glu; Xaa₁₀ is Ala, Leu, Ile, Val, or Met; Xaa₁₁ is Ala or Ser; Xaa₁₂ is Ala or Lys; Xaa₁₃ is Ala or Gln; Xaa₁₄ is Ala, Leu, Ile, Val or Met; Xaa₁₅ is Ala or Glu; Xaa₁₆ is Ala or Glu; Xaa₁₇ is Ala or Glu; Xaa₁₉ is Ala or Val; Xaa₂₀ is Ala or Arg; Xaa₂₁ is Ala or Leu; Xaa₂₂ is Ala, Phe, Tyr; Xaa₂₃ is Ile, Val, Leu, or Met; Xaa₂₄ is Ala, Glu or Asp; Xaa₂₅ is Ala, Trp, Phe, Tyr; Xaa₂₆ is Ala or Leu; Xaa₂₇ is Ala or Lys; Xaa₂₈ is Ala or Asn. In one embodiment, the peptide component of Formula (IV) further includes a moiety Z₁ bonded at the C-terminal, wherein Z₁ is Gly, Gly-Gly (SEQ ID NO:10), Gly-Gly-Xaa₃₁ (SEQ ID NO:11), Gly-Gly-Xaa₃₁-Ser (SEQ ID NO:12), Gly-Gly-Xaa₃₁-Ser-Ser (SEQ ID NO:13), Gly-Gly-Xaa₃′-Ser-Ser-Gly (SEQ ID NO:14), Gly-Gly-Xaa₃₁-Ser-Ser-Gly-Ala (SEQ ID NO:15), Gly-Gly-Xaa₃′-Ser-Ser-Gly-Ala-Xaa₃₆ (SEQ ID NO:16), Gly-Gly-Xaa₃₁-Ser-Ser-Gly-Ala-Xaa₃₆-Xaa₃₇ (SEQ ID NO:17), or Gly-Gly-Xaa₃ᵢ-Ser-Ser-Gly-Ala-Xaa₃₆-Xaa₃₇-Xaa₃₈ (SEQ ID NO:18), and wherein Xaa₃₁, Xaa₃₆, Xaa₃₇ and Xaa₃₈ are independently Pro or are absent. It is expressly contemplated that each exendin analog agonist can be a C-terminal acid or C-terminal amine. In any and each of the exendin analogs described herein, also specifically contemplated are those wherein a replacement for the histidine corresponding to Xaa₁ is made with any of D-histidine, desamino-histidine, 2-amino-histidine, beta-hydroxy-histidine, homohistidine. N-alpha-acetyl-histidine, alpha-fluoromethyl-histidine, alpha-methyl-histidine, 3-pyridylalanine, 2-pyridylalanine, 4-pyridylalanine, 4-imidazoacetyl, des-amino-histidyl (imidazopropionyl), beta-hydroxy-imidazopropionyl, N-dimethyl-histidyl or beta-carboxy-imidazopropionyl. Further specifically contemplated herein are exendin analogs described herein wherein a replacement for the glycine at Xaa2 is made with any of D-Ala, Val, Leu, Lys, Aib, (1-amino cyclopropyl) carboxylic acid, (1-aminocyclobutyl) carboxylic acid, 1-aminocyclopentyl)carboxylic acid, (1-aminocyclohexyl)carboxylic acid, (1-aminocycloheptyl)carboxylic acid, or (1-amino cyclooctyl)carboxylic acid.

In one embodiment, exemplary compounds include those of the above formula wherein: Xaa₁ is His or Arg; Xaa₂ is Gly or Ala; Xaa₃ is Asp or Glu; Xaa₅ is Ala or Thr; Xaa₆ is Ala or Phe; Xaa₇ is Thr or Ser; Xaa₈ is Ala, Ser or Thr; Xaa₉ is Asp or Glu; Xaa₁₀ is Ala, or Leu; Xaa₁₁ is Ala or Ser; Xaa₁₂ is Ala or Lys; Xaa₁₃ is Ala or Gln; Xaa₁₄ is Ala or Leu; Xaa₁₅ is Ala or Glu; Xaa₁₆ is Ala or Glu; Xaa₁₇ is Ala or Glu; Xaa₁₉ is Ala or Val; Xaa₂₀ is Ala or Arg; Xaa₂₁ is Ala or Leu; Xaa₂₂ is Phe; Xaa₂₃ is Ile, Val; Xaa₂₄ is Ala, Glu or Asp; Xaa₂₅ is Ala, Trp or Phe; Xaa₂₆ is Ala or Leu; Xaa₂₇ is Ala or Lys; Xaa₂₈ is Ala or Asn; Z₁ is absent, Gly, Gly-Gly, Gly-Gly-Xaa₃₁, Gly-Gly-Xaa₃₁-Ser, Gly-Gly-Xaa₃₁-Ser-Ser, Gly-Gly-Xaa₃₁-Ser-Ser-Gly, Gly-Gly-Xaa₃₁-Ser-Ser-Gly-Ala, Gly-Gly-Xaa₃₁-Ser-Ser-Gly-Ala-Xaa₃₆, Gly-Gly-Xaa₃₁-Ser-Ser-Gly-Ala-Xaa₃₆-Xaa₃₇, Gly-Gly-Xaa₃₁-Ser-Ser-Gly-Ala-Xaa₃₆-Xaa₃₇-Xaa₃₈; wherein Xaa₃₁, Xaa₃₆, Xaa₃₇ and Xaa₃₈ being independently Pro or is absent, provided that no more than three of Xaa₃, Xaa₅, Xaa₆, Xaa₈, Xaa₁₀, Xaa₁₁, Xaa₁₂, Xaa₁₃, Xaa₁₄, Xaa₁₅, Xaa₁₆, Xaa₁₇, Xaa₁₉, Xaa₂₀, Xaa₂₁, Xaa₂₄, Xaa₂₅, Xaa₂₆, Xaa₂₇ and Xaa₂₈ are Ala. It is expressly contemplated that each exendin analog agonist can be a C-terminal acid or C-terminal amine. In any and each of the exendin analogs described above, also specifically contemplated are those wherein a replacement for the histidine corresponding to position Xaa1 is made with any of D-histidine, desamino-histidine, 2-amino-histidine, beta-hydroxy-histidine, homohistidine. N-alpha-acetyl-histidine, alpha-fluoromethyl-histidine, alpha-methyl-histidine, 3-pyridylalanine, 2-pyridylalanine, 4-pyridylalanine, 4-imidazoacetyl, des-amino-histidyl (imidazopropionyl), beta-hydroxy-imidazopropionyl, N-dimethyl-histidyl or beta-carboxy-imidazopropionyl. Further specifically contemplated herein are exendin analogs described herein wherein a replacement for the glycine at Xaa₂ is made with any of D-Ala, Val, Leu, Lys, Aib, (1-aminocyclopropyl) carboxylic acid, (1-amino cyclobutyl)carboxylic acid, 1-aminocyclopentyl)carboxylic acid, (1-aminocyclohexyl) carboxylic acid, (1-amino cycloheptyl)carboxylic acid, or (1-aminocyclooctyl)carboxylic acid.

Other exemplary exendin analogs include those set forth in WO 99/25727 identified therein as compounds 2-23. According to another embodiment, provided are compounds where Xaa₁₄ is Leu, Ile, or Val, more preferably Leu, and/or Xaa₂₅ is Trp, Phe or Tyr, more preferably Trp or Phe. It is believed that these compounds will be less susceptive to oxidative degradation, both in vitro and in vivo, as well as during synthesis of the compound.

Additional examples of exendin analogs suitable for the LDDHCs described herein include those described in U.S. Pat. No. 6,528,486 published Mar. 4, 2003 (incorporated herein by reference and for all purposes). Specifically, exendin analogs include those consisting of an exendin or exendin analog having at least 90% homology to Exendin-4 having optionally between one and five deletions at positions 34-39, and a C-terminal extension of a peptide sequence of 4-20 amino acid units covalently bound to said exendin wherein each amino acid unit in said peptide extension sequence is selected from the group consisting of Ala, Leu, Ser, Thr, Tyr, Asn, Gln, Asp, Glu, Lys, Arg, His, and Met. More preferably the extension is a peptide sequence of 4-20 amino acid residues, e.g., in the range of 4-15, more preferably in the range of 4-10 in particular in the range of 4-7 amino acid residues, e.g., of 4, 5, 6, 7, 8 or 10 amino acid residues, where 6 amino acid residues are preferred. Most preferably, according to U.S. Pat. No. 6,528,486, the extension peptide contains at least one Lys residue, and is even more preferably from 3 to 7 lysines and even most preferably 6 lysines.

Further exemplary exendin analogs useful for the LDDHCs described herein include: IIGEGTFTSDLSKQMEEEAVRLFIEWLKNGGPSSGAPPSKKKKKK [des-³⁶Pro]Exendin-4(1-39)-Lys₆) (SEQ ID NO:19); KKKKKKHGEGTFTSDLSKQMEEEAVRLFIEWLKNGGPSSGAPPSKKKKKK (H-Lys₆-[des ³⁶Pro]Exendin-4(1-39)-Lys₆) (SEQ ID NO:20); HGEGTFTSDLSKQMEEEAVRLFIEWLWLKNGGPSSGAS (H-[des ³⁶Pro, ^{37,38}Pro]Exendin-4(1-39)-NH₂) (SEQ ID NO:21); KKKKKKHGEGTFTSDLSKQMEEEAVRLFIEWLKNGGPSSGAS (H-(Lys)₆-[des ³⁶Pro, ^{37,38}Pro]Exendin-4(1-39) (SEQ ID NO:22); NEEEEEHGEGTFTSDLSKQMEEEAVRLFIEWLKNGGPSSGAS (H-Asn-(Glu)₅-[des ³⁶Pro, ^{37,38}Pro]Exendin-4(1-39) (SEQ ID NO:23); HGEGTFTSDLSKQMEEEAVRLFIEWLKNGGPSSGASKKKKKK ([des ³⁶Pro, ^{37,38}Pro]Exendin-4(1-39)-(Lys)₆) (SEQ ID NO:24); KKKKKKHGEGTFTSDLSKQMEEEAVRLFIEWLKNGGPSSGASKKKKKK (H-(Lys)₆-[des ³⁶Pro, ^{37,38}Pro]Exendin-4(1-39)-(Lys)₆) (SEQ ID NO:25); and DEEEEEHGEGTFTSDLSKQMEEEAVRLFIEWLKNGGPSSGASKKKKKK (Asn-(Glu)₅-[des ³⁶Pro, ^{37,38}Pro]Exendin-4(1-39)-(Lys)₆) (SEQ ID NO:26).

As customary in the art, repetition of an amino acid can be indicated by a subscripted number setting forth the number of repetitions; i.e., Lys₆, (Lys)₆ and the like refer to hexylysyl (SEQ ID NO:27). As will be clear to the skilled artisan, in some contexts a subscripted number can also indicate the position of a residue within a sequence; e.g., “AA₁ AA₂ AA₃” refers to amino acids 1-3 of a polypeptide sequence. In any and each of the exendin analogs described above, specifically contemplated are those wherein a replacement for the histidine corresponding to position 1 is made with any of D-histidine, desamino-histidine, 2-amino-histidine, beta-hydroxy-histidine, homohistidine. N-alpha-acetyl-histidine, alpha-fluoromethyl-histidine, alpha-methyl-histidine, 3-pyridylalanine, 2-pyridylalanine, 4-pyridylalanine, 4-imidazoacetyl, des-amino-histidyl (or imidazopropionyl), beta-hydroxy-imidazopropionyl, N-dimethyl-histidyl or beta-carboxy-imidazopropionyl. Further specifically contemplated herein are exendin analogs described herein wherein a replacement for the glycine at position 2 is made with any of D-Ala, Val, Leu, Lys, Aib, (1-aminocyclopropyl)carboxylic acid, (1-aminocyclobutyl)carboxylic acid, 1-aminocyclopentyl)carboxylic acid, (1-amino cyclohexyl)carboxylic acid, (1-aminocycloheptyl) carboxylic acid, or (1-aminocyclooctyl) carboxylic acid.

Further examples of exendin analogs useful for the LDDHCs described herein are those described in published PCT application WO2004035623 (incorporated herein by reference and for all purposes), particularly those comprised of naturally-occurring amino acids, which describes exendin analogs having at least one modified amino acid residue particularly at positions ¹³Gln, ¹⁴Met, ²⁵Trp or ²⁸Asn with reference to the corresponding positions of Exendin-4(1-39). Additional such analogs further include a 1-7 amino acid C-terminal extension that includes at least one Lys amino acid and more preferably at least five Lys amino acid units such as six or seven Lys amino acid units.

Yet further examples of exendin analogs useful for the LDDHCs described herein are those described in published PCT application WO/2010/120476, (incorporated herein by reference and for all purposes), which describes exendin analogs having modified amino acid residues in the N-terminal portion of an exendin or exendin analog to create a high beta-turn characteristic in that region. For example, analogs are designed to mimic amino acid residues His₁ Gly₂ Glu₃ by creating a conformationally constrained region, include exendin analogs containing a thiazolidine-proline peptide mimetic at His₁ Gly₂ Glu₃, which can be used as a modification in Exendin-4 or other analogs described herein.

In any and each of the exendins, exendin analogs and formulae described herein, specifically contemplated are those wherein a replacement for the histidine corresponding to position 1 is made with any of D-histidine, desamino-histidine, 2-amino-histidine, beta-hydroxy-histidine, homohistidine. N-alpha-acetyl-histidine, alpha-fluoromethyl-histidine, alpha-methyl-histidine, 3-pyridylalanine, 2-pyridylalanine, 4-pyridylalanine, 4-imidazoacetyl, des-amino-histidyl (imidazopropionyl), beta-hydroxy-imidazopropionyl, N-dimethyl-histidyl or beta-carboxy-imidazopropionyl. For example, contemplated exendin analogs include compounds wherein the His¹ position is modified are (4-imidazoacetyl) Exendin-4, (des-amino-histidyl) Exendin-4 (or (imidazopropionyl) Exendin-4), (beta-hydroxy-imidazopropionyl) Exendin-4, (N-dimethyl-histidyl) Exendin-4 and (beta-carboxy-imidazopropionyl) Exendin-4. Further specifically contemplated herein are exendins or exendin analogs described herein wherein a replacement for the glycine at position 2 is made with any of D-Ala, Val, Leu, Lys, Aib, (1-aminocyclopropyl)carboxylic acid, (1-aminocyclobutyl)carboxylic acid, 1-aminocyclopentyl)carboxylic acid, (1-amino cyclohexyl)carboxylic acid, (1-aminocycloheptyl)carboxylic acid, or (1-aminocyclooctyl) carboxylic acid.

Amylin Agonist Compounds.

“Amylin agonist compounds” include native amylin peptides, amylin analog peptides, and other compounds (e.g., small molecules) that have amylin agonist activity. The “amylin agonist compounds” can be derived from natural sources, can be synthetic, or can be derived from recombinant DNA techniques. Amylin agonist compounds have amylin agonist receptor binding activity and may comprise amino acids (e.g., natural, unnatural, or a combination thereof), peptide mimetics, chemical moieties, and the like. The terms “mimetic,” peptidomimetic,” “peptide mimetic” and the like refer in the customary sense to a compound containing non-peptidic structural elements that is capable of agonizing or antagonizing the biological action(s) of a natural parent peptide. The skilled artisan will recognize amylin agonist compounds using amylin receptor binding assays or by measuring amylin agonist activity in soleus muscle assays. Amylin agonist compounds can have an IC₅₀ of about 200 nM or less, about 100 nM or less, or about 50 nM or less, in an amylin receptor binding assay, such as that described herein, in U.S. Pat. No. 5,686,411, and US Publication No. 2008/0176804, the disclosures of which are incorporated by reference herein in their entireties and for all purposes. The term “IC₅₀” refers in the customary sense to the half maximal inhibitory concentration of a compound inhibiting a biological or biochemical function. Accordingly, in the context of receptor binding studies, IC₅₀ refers to the concentration of a test compound which competes half of a known ligand from a specified receptor. Amylin agonist compounds can have an EC₅₀ of about 20 nM or less, about nM 15 or less, about nM 10 or less, or about nM 5 or less in a soleus muscle assay, such as that described herein and in U.S. Pat. No. 5,686,411. The term “EC₅₀” refers in the customary sense to the effective concentration of a compound which induces a response halfway between a baseline response and maximum response, as known in the art. Amylin agonist compound can have at least 90% or 100% sequence identity to [^{25,28,29}Pro] human-amylin (pramlintide). The amylin agonist compound can be a pep tide chimera of amylin (e.g., human amylin, rat amylin, and the like) and calcitonin (e.g., human calcitonin, salmon calcitonin, and the like), such as davalintide. Further suitable and exemplary amylin agonist compounds are also described in US Publication No. 2008/0274952, the disclosure of which is incorporated by reference herein in its entirety and for all purposes.

Amylins.

In certain embodiments, P₂ is an amylin, amylin analog or derivative thereof. In one embodiment, P₂ is an amylin. In one embodiment, P₂ is an amylin analog. In one embodiment, P₂ is a derivative of an amylin. Amylins, amylin analogs and derivatives thereof suitable for use in the compounds and methods described herein include the compounds described in WO 2007/022123 (PCT/US2006/031724, filed Aug. 11, 2006), incorporated herein by reference and for all purposes. Amylin is a peptide hormone synthesized by pancreatic 13-cells that is co-secreted with insulin in response to nutrient intake. The sequence of amylin is highly preserved across mammalian species, with structural similarities to calcitonin gene-related peptide (CGRP), the calcitonins, the intermedins, and adrenomedullin, as known in the art. The glucoregulatory actions of amylin complement those of insulin by regulating the rate of glucose appearance in the circulation via suppression of nutrient-stimulated glucagon secretion and slowing gastric emptying. In insulin-treated patients with diabetes, pramlintide, a synthetic and equipotent analogue of human amylin, reduces postprandial glucose excursions by suppressing inappropriately elevated postprandial glucagon secretion and slowing gastric emptying. The sequences of rat amylin, human amylin and pramlintide follow:

(SEQ ID NO: 28)
KCNTATCATQRLANFLVRSSNNLGPVLPPTNVGSNTY;
(SEQ ID NO: 29)
KCNTATCATQRLANFLVHSSNNFGAILSSTNVGSNTY;
(SEQ ID NO: 30)
KCNTATCATQRLANFLVHSSNNFGPILPPTNVGSNTY.

In one embodiment, the P₂ polypeptide component includes an amino acid sequence of residues 1-37 (SEQ ID NO:31) of Formula (II) following, wherein up to 55% of the amino acids set forth in Formula (II) may be deleted or substituted with a different amino acid:

(II)
X′-Xaa¹-Cys²-Asn³-Thr⁴-Ala⁵-Thr⁶-Cys⁷-Ala⁸-Thr⁹-
Gln¹⁰-Arg¹¹-Leu¹²-Ala¹³-Asn¹⁴-Phe¹⁵-Leu¹⁶-Val¹⁷-
His¹⁸-Ser¹⁹-Ser²⁰-Xaa²¹-Asn²²-Phe²³-Xaa²⁴-Xaa²⁵-
Xaa²⁶-Xaa²⁷-Xaa²⁸-Xaa²⁹-Thr³⁰-Xaa³¹-Val³²-Gly³³-
Ser³⁴-Asn³⁵-Thr³⁶-Tyr³⁷-X.

In Formula (II), X′ is hydrogen, an N-terminal capping group, a bond to a peptidic or non-peptidic water-soluble polymeric spacer, or a linker to a peptidic or non-peptidic water-soluble polymeric spacer. Xaa¹ is Lys or a bond, Xaa²¹ is Lys, Cys, or Asn, Xaa²⁴ is Lys, Cys, or Gly, Xaa²⁵ is Lys, Cys, or Pro, Xaa²⁶ is Lys, Cys, or Ile, Xaa²⁷ is Lys, Cys, or Leu, Xaa²⁸ is Lys, Cys, or Pro, Xaa²⁹ is Lys, Cys, or Pro and Xaa³¹ is Lys, Cys, or Asn.

Further regarding Formula (II), the variable X represents a C-terminal functionality (e.g., a C-terminal cap). Accordingly, X is substituted or unsubstituted amino, substituted or unsubstituted alkylamino, substituted or unsubstituted dialkylamino, substituted or unsubstituted cycloalkylamino, substituted or unsubstituted arylamino, substituted or unsubstituted aralkylamino, substituted or unsubstituted alkyloxy, substituted or unsubstituted aryloxy, substituted or unsubstituted aralkyloxy, hydroxyl, a bond to a peptidic or non-peptidic water-soluble polymeric spacer, or a linker to a peptidic or non-peptidic water-soluble polymeric spacer. In one embodiment, the peptidic or non-peptidic water-soluble polymeric spacer is covalently linked, optionally through a linker, to a side chain of a linking amino acid residue, X′ or X. In one embodiment, the peptidic or non-peptidic water-soluble polymeric spacer is covalently linked, optionally through a linker, to a backbone atom of the polypeptide component. If the C-terminal of the polypeptide component with the sequence of residues 1-37 of Formula (II) is capped with a functionality X, then X is preferably amine thereby forming a C-terminal amide. The N-terminal of polypeptide components described herein, including the polypeptide component according to Formula (II), can be covalently linked to a variety of functionalities including, but not limited to, the acetyl group. The term “N-terminal capping group” refers in the customary sense to a moiety covalently bonded to the N-terminal nitrogen of a polypeptide, e.g., substituted or unsubstituted acyl, substituted or unsubstituted acyloxy, Schiff's bases, and the like, as known in the art. In one embodiment, the N-terminal functionality X′ is an amine-protecting group as known in the art, preferably Fmoc.

In one embodiment, up to 5%, 10%, 15%, 20%, 25%, 30%, 35%, 40%, 45%, 50% or even 55% of the amino acids of residues 1-37 of Formula (II) are deleted or substituted in a polypeptide component according to Formula (II). In one embodiment, the polypeptide component has 0, 1, 2, 3, 4, 5, 6, 7, 8, 9, 10, 11, 12, 13, 14, 15, 16, 17, 18, 19 or even 20 amino acid substitutions relative to the amino acid sequence set forth in Formula (II). In one embodiment, the polypeptide component has 0, 1, 2, 3, 4, 5, 6, 7, 8, 9, 10, 11, 12, 13, 14, 15, 16, 17, 18 or even 19 deletions relative to the amino acid sequence set forth in Formula (II).

In one embodiment, the polypeptide component of the polypeptide conjugate has a sequence which has a defined sequence identity with respect to the residues 1-37 of the amino acid sequence according to Formula (II).

In one embodiment, the sequence identity between a polypeptide component described herein and residues 1-37 of Formula (II) is 45%, 50%, 55%, 60%, 65%, 70%, 75%, 80%, 85%, 90%, 95% or even higher. In one embodiment, the sequence identity between a polypeptide component described herein and residues 1-37 of Formula (II) is in the range 45%-50%, 50%-60%, 60%-70%, 70%-80%, 80%-90%, or 90%-100%. In one embodiment, up to 55%, 50%, 45%, 40%, 35%, 30%, 25%, 20%, 15%, 10%, 5% or even less of the amino acids set forth in residues 1-37 of Formula (II) may be deleted or substituted with a different amino acid. In one embodiment, the sequence identity is within the range 75%-100%. In one embodiment, the sequence identity is within the range 75%-90%. In one embodiment, the sequence identity is within the range 80%-90%. In one embodiment, the sequence identity is at least 75%. In one embodiment, the sequence identity is at least 90%. In one embodiment, the polypeptide component of the conjugate has the sequence of residues 1-37 of Formula (II).

In one embodiment, the polypeptide component has the sequence of Cmpd 12. In one embodiment, the polypeptide component has the sequence of Cmpd 6. In one embodiment, the polypeptide component has one or more conservative amino acid substitutions with respect to the sequence of Formula (II). “Conservative amino acid substitution” refers in the customary sense to substitution of amino acids having similar biochemical properties at the side chain (e.g., hydrophilicity, hydrophobocity, charge type, van der Waals radius, and the like). “Non-conservative amino acid substitution” refers in the customary sense to substitution of amino acids having dissimilar biochemical properties at the side chain.

It is understood that in the calculation of sequence identity with respect to any of the polypeptide components set forth herein (e.g., as found in residues 1-37 of Formula (II)), the sequence to be compared is taken over the amino acids disclosed therein, irrespective of any N-terminal (i.e., X′) or C-terminal (i.e., X) functionality present. It is further understood that the presence of a peptidic or non-peptidic water-soluble polymeric spacer covalently linked to the side chain of an amino acid is immaterial to the calculation of sequence identity. For example, a lysine substituted at any position of Formula (I) and additionally bonded, optionally through a linker with a peptidic or non-peptidic water-soluble polymeric spacer, is a lysine for purposes of sequence identity calculation.

Polypeptides including the sequence of residues 1-37 of Formula (II) can be considered to be chimeric combinations of amylin and calcitonin, or analogs thereof.

In one embodiment, there is provided a polypeptide conjugate, which includes a derivative of pramlintide (SEQ ID NO:30) or an analog thereof, wherein the amino acid residue in position 1 is absent (i.e., des-Lys¹) and an amino acid residue in position 2 to 37 has been substituted with a lysine residue or cysteine residue, and wherein the lysine residue or cysteine residue is linked to a peptidic or non-peptidic water-soluble polymeric spacer, e.g., a polyethylene glycol polymer, optionally via a linker, wherein the amino acid numbering conforms with the amino acid numbering of pramlintide. In one embodiment, the peptidic or non-peptidic water soluble polymeric spacer is a polyethylene glycol polymer.

In another embodiment, there is provided a polypeptide conjugate, which includes a derivative of pramlintide or an analog thereof, wherein the amino acid residue in position 1 is absent (i.e., des-Lys¹) and wherein an amino acid residue in any one of position 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 31, 32, 33, 34, 35, 36, or 37 is substituted with a lysine residue, and wherein the lysine residue is linked to a peptidic or non-peptidic water-soluble polymeric spacer, e.g., a polyethylene glycol polymer, optionally via a linker. In one embodiment, the peptidic or non-peptidic water soluble polymeric spacer is a polyethylene glycol polymer.

In another embodiment, there is provided a polypeptide conjugate, which includes a derivative of pramlintide or an analog thereof, wherein the amino acid residue in position 1 is absent (i.e., des-Lys¹) and wherein an amino acid residue in any one of position 21, 24-29, or 31 is substituted with a lysine residue, and wherein the lysine residue is linked to a peptidic or non-peptidic water-soluble polymeric spacer, e.g., a polyethylene glycol polymer, optionally via a linker. In one embodiment, the peptidic or non-peptidic water soluble polymeric spacer is a polyethylene glycol polymer.

In another embodiment, there is provided a polypeptide conjugate, which includes a derivative of pramlintide or an analog thereof, wherein the amino acid residue in position 1 is absent (i.e., des-Lys¹) and wherein an amino acid residue in position 21 is substituted with a lysine residue, and wherein the lysine residue is linked to a peptidic or non-peptidic water-soluble polymeric spacer, e.g., a polyethylene glycol polymer, optionally via a linker. In one embodiment, the peptidic or non-peptidic water soluble polymeric spacer is a polyethylene glycol polymer.

In another embodiment, there is provided a polypeptide conjugate, which includes a derivative of pramlintide or an analog thereof, wherein the amino acid residue in position 1 is absent (i.e., des-Lys¹) and wherein an amino acid residue in position 24 is substituted with a lysine residue, and wherein the lysine residue is linked to a peptidic or non-peptidic water-soluble polymeric spacer, e.g., a polyethylene glycol polymer, optionally via a linker. In one embodiment, the peptidic or non-peptidic water soluble polymeric spacer is a polyethylene glycol polymer.

In another embodiment, there is provided a polypeptide conjugate, which includes a derivative of pramlintide or an analog thereof, wherein the amino acid residue in position 1 is absent (i.e., des-Lys¹) and wherein an amino acid residue in position 25 is substituted with a lysine residue, and wherein the lysine residue is linked to a peptidic or non-peptidic water-soluble polymeric spacer, e.g., a polyethylene glycol polymer, optionally via a linker. In one embodiment, the peptidic or non-peptidic water soluble polymeric spacer is a polyethylene glycol polymer.

In another embodiment, there is provided a polypeptide conjugate, which includes a derivative of pramlintide or an analog thereof, wherein the amino acid residue in position 1 is absent (i.e., des-Lys¹) and wherein an amino acid residue in position 26 is substituted with a lysine residue, and wherein the lysine residue is linked to a peptidic or non-peptidic water-soluble polymeric spacer, e.g., a polyethylene glycol polymer, optionally via a linker. In one embodiment, the peptidic or non-peptidic water soluble polymeric spacer is a polyethylene glycol polymer.

In another embodiment, there is provided a polypeptide conjugate, which includes a derivative of pramlintide or an analog thereof, wherein the amino acid residue in position 1 is absent (i.e., des-Lys¹) and wherein an amino acid residue in position 27 is substituted with a lysine residue, and wherein the lysine residue is linked to a peptidic or non-peptidic water-soluble polymeric spacer, e.g., a polyethylene glycol polymer, optionally via a linker. In one embodiment, the peptidic or non-peptidic water soluble polymeric spacer is a polyethylene glycol polymer.

In another embodiment, there is provided a polypeptide conjugate, which includes a derivative of pramlintide or an analog thereof, wherein the amino acid residue in position 1 is absent (i.e., des-Lys¹) and wherein an amino acid residue in position 28 is substituted with a lysine residue, and wherein the lysine residue is linked to a peptidic or non-peptidic water-soluble polymeric spacer, e.g., a polyethylene glycol polymer, optionally via a linker. In one embodiment, the peptidic or non-peptidic water soluble polymeric spacer is a polyethylene glycol polymer.

In another embodiment, there is provided a polypeptide conjugate, which includes a derivative of pramlintide or an analog thereof, wherein the amino acid residue in position 1 is absent (i.e., des-Lys¹) and wherein an amino acid residue in position 29 is substituted with a lysine residue, and wherein the lysine residue is linked to a peptidic or non-peptidic water-soluble polymeric spacer, e.g., a polyethylene glycol polymer, optionally via a linker. In one embodiment, the peptidic or non-peptidic water soluble polymeric spacer is a polyethylene glycol polymer.

In another embodiment, there is provided a polypeptide conjugate, which includes a derivative of pramlintide or an analog thereof, wherein the amino acid residue in position 1 is absent (i.e., des-Lys¹) and wherein an amino acid residue in position 31 is substituted with a lysine residue, and wherein the lysine residue is linked to a peptidic or non-peptidic water-soluble polymeric spacer, e.g., a polyethylene glycol polymer, optionally via a linker. In one embodiment, the peptidic or non-peptidic water soluble polymeric spacer is a polyethylene glycol polymer.

In another aspect, there is provided a long-duration dual hormone conjugate (LDDHC) compound having the structure of Formula I

P₁-L₁-PS-L₂-P₂ (I)

wherein P₁ has a first biological activity and is an exendin, exendin analog or derivative thereof; P₂ has a second biological activity and is an amylin, amylin analog or derivative thereof; L₁ and L₂ are independently a bond or a linker; and PS is a water-soluble polymeric spacer having a molecular weight in the range 30-80 kDa; wherein the compound exhibits the first biological activity in a biological assay, and the compound exhibits the second biological activity in a biological assay.

In one embodiment, the exendin, exendin analog or derivative thereof is exendin-4, exendin-4 analog or derivative thereof. In one embodiment, the exendin, exendin analog or derivative thereof is exendin-4. In one embodiment, the exendin, exendin analog or derivative thereof is an exendin-4 analog. In one embodiment, the exendin, exendin analog or derivative thereof is an exendin-4 derivative. A most preferred compound of Formula I is Cmpd 14.

In one embodiment, P₂ includes an amino acid sequence of residues 1-37 (SEQ ID NO:31) of Formula (II):

(II)
X′-Xaa¹-Cys²-Asn³-Thr⁴-Ala⁵-Thr⁶-Cys⁷-Ala⁸-Thr⁹-
Gln¹⁰-Arg¹¹-Leu¹²-Ala¹³-Asn¹⁴-Phe¹⁵-Leu¹⁶-Val¹⁷-
His¹⁸-Ser¹⁹-Ser²⁰-Xaa²¹-Asn²²-Phe²³-Xaa²⁴-Xaa²⁵-
Xaa²⁶-Xaa²⁷-Xaa²⁸-Xaa²⁹-Thr³⁶-Xaa³¹-Val³²-Gly³³-
Ser³⁴-Asn³⁵-Thr³⁶-Tyr³⁷-X

wherein up to 55% of the amino acids set forth in Formula (II) may be deleted or substituted with a different amino acid; wherein X′ is hydrogen, an N-terminal capping group, a bond to PS, or a linker to PS; Xaa¹ is Lys or a bond; Xaa²¹ is Lys, Cys, or Asn; Xaa²⁴ is Lys, Cys, or Gly; Xaa²⁵ is Lys, Cys, or Pro; Xaa²⁶ is Lys, Cys, or Ile; Xaa²⁷ is Lys, Cys, or Leu; Xaa²⁸ is Lys, Cys, or Pro; Xaa²⁹ is Lys, Cys, or Pro; Xaa³¹ is Lys, Cys, or Asn.; and X is optional, and if present is substituted or unsubstituted amino, substituted or unsubstituted alkylamino, substituted or unsubstituted dialkylamino, substituted or unsubstituted cycloalkylamino, substituted or unsubstituted arylamino, substituted or unsubstituted aralkylamino, substituted or unsubstituted alkyloxy, substituted or unsubstituted aryloxy, substituted or unsubstituted aralkyloxy, hydroxyl, a bond to PS, or a linker to PS; wherein the PS is covalently linked, optionally through a linker, to a side chain of a linking amino acid residue, X′ or X.

In one embodiment, P₂ includes an amino acid sequence of residues 1-37 (SEQ ID NO:31) of Formula (III):

(III)
Xaa¹-Cys²-Asn³-Thr⁴-Ala⁵-Thr⁶-Cys⁷-Ala⁸-Thr⁹-
Gln¹⁰-Arg¹¹-Leu¹²-Ala¹³-Asn¹⁴-Phe¹⁵-Leu¹⁶-Val¹⁷-
His¹⁸-Ser¹⁹-Ser²⁰-Xaa²¹-Asn²²-Phe²³-Xaa²⁴-Xaa²⁵-
Xaa²⁶-Xaa²⁷-Xaa²⁸-Xaa²⁹-Thr³⁰-Xaa³¹-Val³²-Gly³³-
Ser³⁴-Asn³⁵-Thr³⁶-Tyr³⁷

wherein up to 55% of the amino acids set forth in Formula (III) may be deleted or substituted with a different amino acid. Further to Formula (III), Xaa¹ is Lys or a bond; Xaa²¹ is Lys, Cys, or Asn; Xaa²⁴ is Lys, Cys, or Gly; Xaa²⁵ is Lys, Cys, or Pro; Xaa²⁶ is Lys, Cys, or Ile; Xaa²⁷ is Lys, Cys, or Leu; Xaa²⁸ is Lys, Cys, or Pro; Xaa²⁹ is Lys, Cys, or Pro; and Xaa³¹ is Lys, Cys, or Asn. In one embodiment, the N-terminal of Formula (III) is covalently bonded to a moiety X′, wherein X′ is an N-terminal capping group, a bond to PS, or a linker to PS. In one embodiment, the C-terminal of Formula (III) is covalently bonded to a moiety X, wherein X is substituted or unsubstituted amino, substituted or unsubstituted alkylamino, substituted or unsubstituted dialkylamino, substituted or unsubstituted cycloalkylamino, substituted or unsubstituted arylamino, substituted or unsubstituted aralkylamino, substituted or unsubstituted alkyloxy, substituted or unsubstituted aryloxy, substituted or unsubstituted aralkyloxy, a bond to PS, or a linker to PS. In one embodiment, PS is covalently linked, optionally through a linker, to a side chain of a linking amino acid residue, X′ or X.

In one embodiment, PS has a molecular weight in the range 30-80 kDa, 30-70 kDa, 30-60 kDa, 35-60 kDa, preferably 35-50 kDa. In one embodiment, PS has a molecular weight of about 40 kDa.

Davalintide.

In certain embodiments, P₂ is davalintide, a davalintide analog or derivative thereof. In one embodiment, P₂ is davalintide. In one embodiment, P₂ is a davalintide analog. In one embodiment, P₂ is a derivative of davalintide. Davalintide, also known as “AC-2307” is a potent amylin agonist useful in the treatment of a variety of disease indications. See WO 2006/083254 and WO 2007/114838, each of which is incorporated by reference herein in its entirety and for all purposes, for davalintide analogs and derivatives suitable for use in the compounds and methods described herein. Davalintide is a chimeric peptide, having an N-terminal loop region of amylin or calcitonin and analogs thereof, an alpha-helical region of at least a portion of an alpha-helical region of calcitonin or analogs thereof or an alpha-helical region having a portion of an amylin alpha-helical region and a calcitonin alpha-helical region or analog thereof, and a C-terminal tail region of amylin or calcitonin. The sequences of human calcitonin, salmon calcitonin and davalintide follow:

(SEQ ID NO: 32)
CGNLSTCMLGTYTQDFNKFHTFPQTAIGVGAP;
(SEQ ID NO: 33)
CSNLSTCVLGKLSQELHKLQTYPRTNTGSGTP;
(SEQ ID NO: 34)
KCNTATCVLGRLSQELHRLQTYPRTNTGSNTY.

In certain embodiments, P₂ includes from 1 to 37 residues. In certain embodiments, P₂ is an amylin, pramlintide or davalintide. In certain embodiments, P₂ is an amylin analog or an analog of pramlintide or davalintide. In certain embodiments, P₂ is a derivative of amylin or an amylin analog, of pramlintide or a pramlintide analog, or of davalintide or a davalintide analog.

Linkers. The terms “linker” and the like, in the context of attachment of polypeptide components and water-soluble polymeric spacer components in LDDHCs described herein, means a divalent species (-L-) covalently bonded in turn to a polypeptide component (e.g., P₁, P₂) having a valency available for bonding and to a water-soluble polymeric spacer (e.g., PS) having a valency available for bonding. The available bonding site on the polypeptide component is conveniently a side chain residue (e.g., lysine, cysteine, aspartic acid, and homologs thereof). In one embodiment, the available bonding site on the polypeptide component is the side chain of a lysine or a cysteine residue. In one embodiment, the available bonding site on the polypeptide component is the N-terminal amine. In one embodiment, the available bonding site on the polypeptide component is the C-terminal carboxylate. As used herein, the term “linking amino acid residue” means an amino acid to which a water-soluble polymeric spacer is attached, optionally through a linker.

In certain embodiments, either one or both of L₁ and L₂ are bonds. When not a bond, the chemical structure of L₁ or L₂ is not critical, since L₁ and L₂ serve primarily as spacers, which can be useful in optimizing pharmacological activity of some embodiments of the LDDHCs described herein. Linkers L₁ and L₂ can be independently the same or different.

In one embodiment, linkers L₁ and L₂ are independently a bond, —C(O)—, —NH—, —O—, —S—, —S—S—, —COO—, —OCONH—, —NHCONH—, substituted or unsubstituted alkylene, substituted or unsubstituted alkenylene, substituted or unsubstituted urethane, substituted or unsubstituted alkylamide, substituted or unsubstituted alkylsulfone, substituted or unsubstituted heteroalkylene, substituted or unsubstituted cycloalkylene, substituted or unsubstituted heterocycloalkylene, substituted or unsubstituted arylene, or substituted or unsubstituted heteroarylene, and the like, as known in the art.

In one embodiment, linkers L₁ and L₂ are independently a bond, R¹-substituted or unsubstituted alkylene, R¹-substituted or unsubstituted alkenylene, R¹-substituted or unsubstituted urethane, R¹-substituted or unsubstituted alkylamide, R¹-substituted or unsubstituted alkylsulfone, R¹-substituted or unsubstituted heteroalkylene, R¹-substituted or unsubstituted cycloalkylene, R¹-substituted or unsubstituted heterocycloalkylene, substituted or unsubstituted arylene, or substituted or unsubstituted heteroarylene. R¹ is R²-substituted or unsubstituted alkyl, R²-substituted or unsubstituted heteroalkyl, R²-substituted or unsubstituted cycloalkyl, R²-substituted or unsubstituted heterocycloalkyl, R²-substituted or unsubstituted aryl, or R²-substituted or unsubstituted heteroaryl. R² is R³-substituted or unsubstituted alkyl, R³-substituted or unsubstituted heteroalkyl, R³-substituted or unsubstituted cycloalkyl, R³-substituted or unsubstituted heterocycloalkyl, R³-substituted or unsubstituted aryl, or R³-substituted or unsubstituted heteroaryl. R³ is unsubstituted alkyl, unsubstituted heteroalkyl, unsubstituted cycloalkyl, unsubstituted heterocycloalkyl, unsubstituted aryl or unsubstituted heteroaryl.

In one embodiment, the linker is a polyfunctional amino acid, for example but not limited to, beta-alanine, lysine and homologs thereof, and aspartic acid and homologs thereof. The term “polyfunctional” in the context of an amino acid refers to a side chain functionality which can react to form a bond, in addition to the amino and/or carboxyl functionalities of the amino acid. Exemplary functionalities of polyfunctional amino acids include amine, carboxyl and sulfhydryl functionalities.

In one embodiment, the linker includes a divalent heteroatom. In one embodiment, the linker is, or includes, —O—, —S—, —S—S—, —COO—, —OCONH—, and —NHCONH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. Representative linkers include —O—, —S—, —S—S—, —COO—, —OCONH—, and —NHCONH—, amide and/or urethane linkage attached to the peptidic or non-peptidic water-soluble polymeric spacer and the polypeptide component.

In one embodiment, the linker results from direct chemical conjugation between an amino acid side chain of a backbone functionality (moiety) of the polypeptide component and a functionality on the water-soluble polymeric spacer. Exemplary of this type of conjugation is the formation of an amide bond achieved by standard solid-phase synthetic methods, as well known in the art. The linkers described herein are exemplary, and linkers within the scope of this invention may be much longer and may include other residues.

In one embodiment, linkers L₁ and L₂ independently have the structure -L_{A}-L₁-, wherein linking elements L_{A} and L_{B} are each independently a divalent heteroatom, —O—, —S—, —S—S—, —COO—, —OCONH—, and —NHCONH—, substituted or unsubstituted alkylene, substituted or unsubstituted heteroalkylene, substituted or unsubstituted cycloalkylene, substituted or unsubstituted heterocycloalkylene, substituted or unsubstituted arylene, substituted or unsubstituted heteroarylene, or a substituted or unsubstituted PEG. In one embodiment, L^{A} and L^{B} are independently —OCO—(CH₂)ₙ—CO—, —O—(CH₂)ₙ—NHCO—, —O—(CH₂)ₙ—, —O—(CH₂)ₙ—CONH—(CH₂)ₙ—, —O—(CH₂)ₙ—, —SO₂—(CH₂)ₙ—, —SO₂—(CH₂)ₙ—,S—, wherein “n” is independently 1-5 at each occurrence. “Linking element” refers to covalently bonded elements of a linker (e.g., L_{A} and L_{B}).

In one embodiment, the linker includes two or more of substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

In one embodiment, linking elements L_{A} and L_{B} are independently a bond, R⁴-substituted or unsubstituted alkylene, R⁴-substituted or unsubstituted alkenylene, R⁴-substituted or unsubstituted urethane, R⁴-substituted or unsubstituted alkylamide, R⁴-substituted or unsubstituted alkylsulfone, R⁴-substituted or unsubstituted heteroalkylene, R⁴-substituted or unsubstituted cycloalkylene, R⁴-substituted or unsubstituted heterocycloalkylene, substituted or unsubstituted arylene, or substituted or unsubstituted heteroarylene. R⁴ is R⁵-substituted or unsubstituted alkyl, R⁵-substituted or unsubstituted heteroalkyl, R⁵-substituted or unsubstituted cycloalkyl, R⁵-substituted or unsubstituted heterocycloalkyl, R⁵-substituted or unsubstituted aryl, or R⁵-substituted or unsubstituted heteroaryl. R⁵ is R⁶-substituted or unsubstituted alkyl, R⁶-substituted or unsubstituted heteroalkyl, R⁶-substituted or unsubstituted cycloalkyl, R⁶-substituted or unsubstituted heterocycloalkyl, R⁶-substituted or unsubstituted aryl, or R⁶-substituted or unsubstituted heteroaryl. R⁶ is unsubstituted alkyl, unsubstituted heteroalkyl, unsubstituted cycloalkyl, unsubstituted heterocycloalkyl, unsubstituted aryl or unsubstituted heteroaryl.

In one embodiment, linkers L₁ and L₂ independently have the structure —OCO—(CH₂)ₙ—CO—, —O—(CH₂)ₙ—NHCO—, —O—(CH₂)ₙ—, —O—(CH₂)ₙ—CONH—(CH₂)ₙ—, —O—(CH₂)ₙ—, —SO₂—(CH₂)ₙ—, —SO₂—(CH₂)ₙ—,S—, wherein “n” is independently 1-5 at each occurrence.

In one embodiment, a substituted group described herein is substituted with at least one substituent group. More specifically, in one embodiment, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, or substituted heteroarylene within a linker or linking element described herein is substituted with at least one substituent group. In other embodiments, at least one or all of these groups are substituted with at least one size-limited substituent group. Alternatively, at least one or all of these groups are substituted with at least one lower substituent group.

In other embodiments of the linkers described herein, each substituted or unsubstituted alkyl is a substituted or unsubstituted C₁-C₂₀) alkyl, each substituted or unsubstituted heteroalkyl is a substituted or unsubstituted 2 to 20 membered heteroalkyl, each substituted or unsubstituted cycloalkyl is a substituted or unsubstituted C₄-C₈ cycloalkyl, each substituted or unsubstituted heterocycloalkyl is a substituted or unsubstituted 4 to 8 membered heterocycloalkyl, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene substituted or unsubstituted C₄-C₈ cycloalkylene, and each substituted or unsubstituted heterocycloalkylene is a substituted or unsubstituted 4 to 8 membered heterocycloalkylene. In one embodiment, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₅-C₇ cycloalkyl, each substituted or unsubstituted heterocycloalkyl is a substituted or unsubstituted 5 to 7 membered heterocycloalkyl, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene substituted or unsubstituted C₅-C₆ cycloalkylene, and each substituted or unsubstituted heterocycloalkylene is a substituted or unsubstituted 5 to 7 membered heterocycloalkylene.

In one embodiment, a water-soluble polymeric spacer is attached to a LDDHC described herein via linkers known in the art, for example but not limited to, the cysteine linked water-soluble polymeric spacer (PS) as shown following.

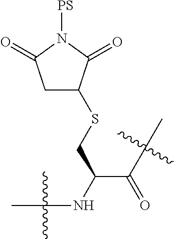

In certain embodiments, a linker may be one or more amino acid residues, typically from about 1 to about 50 amino acid residues, preferably 1-20 amino acid residues, and more preferably about 1-10 amino acid residues. Preferably, but not necessarily, the amino acid residues in the linker are from among the twenty canonical (i.e., physiologic) amino acids, more preferably, cysteine, glycine, alanine, proline, asparagine, glutamine, and/or serine. Even more preferably, a peptidyl linker is made up of a majority of amino acids that are sterically unhindered, such as glycine, serine, and alanine linked by a peptide bond. It is also desirable that, if present, a peptidyl linker be selected that avoids rapid proteolytic turnover in circulation in vivo. Some of these amino acids may be glycosylated, as is well understood by those in the art.

In other embodiments, the amino acids of the linker are selected from glycine, alanine, proline, asparagine, glutamine, and lysine. Preferably, a linker is made up of a majority of amino acids that are sterically unhindered, such as glycine and alanine. Thus, preferred linkers include polyglycines, polyserines, and polyalanines, or combinations of any of these. In certain embodiments, beta-amino acids (e.g., β-ala) as known in the art are included in the linker. Exemplary linkers include (β-ala)ₙ, where n is 1 to 20, preferably 1 to 10, more preferably 1 to 4.

Some exemplary peptidyl linkers are poly(Gly)₁₋₈, particularly (Gly)₃, (Gly)₄(SEQ ID NO:35), (Gly)₅ (SEQ ID NO:36), (Gly)₆ (SEQ ID NO:37) and (Gly), (SEQ ID NO: 38), as well as polymers of (Gly)₄Ser (SEQ ID NO:39), polymers of (Gly-Ala)₂₋₄ (SEQ ID NO:40) and poly(Ala)₂₋₈ (SEQ ID NO:41). Other specific examples of peptidyl linkers include (Gly)₅Lys (SEQ ID NO: 42), and (Gly)₅LysArg (SEQ ID NO: 43).

Other specific examples of linkers include: (Gly)₃Lys(Gly)₄ (SEQ ID NO:44); (Gly)₃AsnGlySer(Gly)₂ (SEQ ID NO:45); (Gly)₃Cys(Gly)₄ (SEQ ID NO:46); and GlyProAsnGlyGly (SEQ ID NO: 47).

Other preferred linkers include GGGGS (SEQ ID NO:39), GGGGSGGGGS (SEQ ID NO:48), GGGGSGGGGSGGGGSGGGGSGGGGS (SEQ ID NO:49) and any linkers used in the working examples hereinafter.

In one embodiment, the linkers include the following peptide linker sequences:

(SEQ ID NO: 50)
GGEGGG,
(SEQ ID NO: 51)
GGEEEGGG,
(SEQ ID NO: 52)
GEEEG,
(SEQ ID NO: 53)
GEEE,
(SEQ ID NO: 54)
GGDGGG,
(SEQ ID NO: 55)
GGDDDGG,
(SEQ ID NO: 56)
GDDDG,
(SEQ ID NO: 57)
GDDD,
(SEQ ID NO: 58)
GGGGSDDSDEGSDGEDGGGGS,
(SEQ ID NO: 59)
WEWEW,
(SEQ ID NO: 60)
FEFEF,
(SEQ ID NO: 61)
EEEWWW,
(SEQ ID NO: 62)
EEEFFF,
(SEQ ID NO: 63)
WWEEEWW,
or
(SEQ ID NO: 64)
FFEEEFF.

In other embodiments, the linker constitutes a potential phosphorylation site, e.g., X₁X₂YX₄X₅G, wherein X_{S}, X₂, X₄ and X₅ are each independently any amino acid residue; X₁X₂SX₄X₅G, wherein X₁, X₂, X₄ and X₅ are each independently any amino acid residue; or X₁X₂TX₄X₅G, wherein X₁, X₂, X₄ and X₅ are each independently any amino acid residue.

In another embodiment, the linker contains a cysteine or homocysteine residue, or other 2-amino-ethanethiol or 3-amino-propanethiol moiety for conjugation to maleimide, iodoacetaamide or thioester, functionalized half-life extending moiety.

Another useful peptidyl linker is a large, flexible linker comprising a random Gly/Ser/Thr sequence, for example: GSGSATGGSGSTASSGSGSATH (SEQ ID NO:65) or HGSGSATGGSGSTASSGSGSAT (SEQ ID NO:66), that is estimated to be about the size of a 1 kDa polyethylene glycol (PEG) molecule. Alternatively, a useful peptidyl linker may be comprised of amino acid sequences known in the art to form rigid helical structures (e.g., Rigid linker: -AEAAAKEAAAKEAAAKAGG-, SEQ ID NO:67). Additionally, a peptidyl linker can also comprise a non-peptidyl segment such as a 6 carbon aliphatic molecule of the formula —(CH₂)₆—. The peptidyl linkers can be altered to form derivatives as described herein.

Optionally, non-peptidyl linkers are also useful for conjugating the PS moiety to a peptide portion of a LDDHC described herein. For example, alkylene linkers such as —NH—(CH₂)ₛ—C(O)—, wherein s=2-20 can be used. In one embodiment, alkylene linkers useful for LDDHCs described herein are R¹-substituted or unsubstituted alkylene, where R¹ is as described herein.

These alkylene linkers may further be substituted by any non-sterically hindering group such as lower alkyl (e.g., C₁-C₆) lower acyl, halogen (e.g., Cl, Br), CN, NH₂, phenyl, etc. Exemplary non-peptidyl linkers are PEG linkers, as known in the art and/or described herein. Preferably such non-peptidyl linkers are independently no more than 0.5 kDa, no more than 1 kDa, and no more than 2 kDa, and are preferably linear.

In one embodiment, the water-soluble polymeric spacer PS in combination with L₁ and L₂ (i.e., L₁-PS-L₂) in Formula (I) has a combined molecular weight in the range 35-85 kDa, 35-75 kDa, 35-60 kDa, preferably 35-50 kDa. In one embodiment, L₁-PS-L₂ has a molecular weight of about 35, 36, 37, 38, 39, 40, 41, 42, 43, 44, 45, 46, 47, 48, 49 or 50 kDa.

Water Soluble Polymer Spacer.

The term “water-soluble polymer spacer” (PS) in the context of LDDHCs of Formula (I) refers to a peptidic or non-peptidic polymer which is sufficiently soluble in aqueous solution to be useful for the methods described herein (e.g., suitable for injection). In one embodiment, the PS is selected such that the LDDHC does not form a depot upon injection, as known in the art. In one embodiment, the PS is selected such that the LDDHC forms a depot upon injection.

In one embodiment, the size range for the water-soluble polymer spacer is 30-80 kDa, 30-70 kDa, 30-60 kDa, 35-60 kDa, and even 35-50 kDa. In one embodiment, the spacer is 20 kDa, 25 kDa, 30 kDa, 35 kDa, 40 kDa, 45 kDa, 50 kDa, 60 kDa, 70 kDa and even 80 kDa. Non-peptidic polymers suitable for use as moiety PS in Formula (I) include a variety of compounds known in the art including, but not limited to, polyethylene glycol (PEG), monomethoxy-polyethylene glycol (mPEG), dextran, cellulose, poly-(N-vinyl pyrrolidone) polyethylene glycol, propylene glycol homopolymers, polypropylene oxide/ethylene oxide co-polymers, polyoxyethylated polyols and polyvinyl alcohol. A linear PEG is a most preferred PS. A linear PEG has provided superior effects compared to a comb-like PEG and a branched PEG of the same molecular weight as the linear PEG (data not shown). A combined molecular weight for PS and L¹ and L², when both or either L¹ or L² are present, is 15-85 kDa, preferably 25-85 kDa, 25-75 kDa, 30-65 kDa and even 30-60 kDa.

PEG molecules useful for derivatization of polypeptides are typically classified into linear, branched and Warwick (i.e., PoIyPEG®) classes of PEGs, as known in the art. Unless expressly indicated to the contrary, the PEG moieties described herein are linear PEGs. The terms “two arm branched,” “Y-shaped” (yPEG) and the like refer to branched PEG moieties, as known in the art. The term “Warwick” in the context of PEGs, also known as “comb” or “comb-type” PEGs, refers to a variety of multi-arm PEGs attached to a backbone, typically poly(methacrylate), as known in the art.

In one embodiment, non-peptidic polymers suitable for use as moiety PS in Formula I include substituted or unsubstituted PEG, substituted or unsubstituted monomethoxy-polyethylene glycol, substituted or unsubstituted dextran, substituted or unsubstituted cellulose, substituted or unsubstituted poly-(N-vinyl pyrrolidone) polyethylene glycol, substituted or unsubstituted propylene glycol homopolymers, substituted or unsubstituted polypropylene oxide/ethylene oxide co-polymers, substituted or unsubstituted polyoxyethylated polyols and substituted or unsubstituted polyvinyl alcohol.

In one embodiment, PS is monovalent. In one embodiment, non-peptidic polymers suitable for use as moiety PS in Formula (I) include substituted alkyl, substituted heteroalkyl, substituted cycloalkyl, substituted heterocycloalkyl, substituted aryl, and substituted heteroaryl, which are in turn substituted with substituted or unsubstituted PEG, substituted or unsubstituted monomethoxy-polyethylene glycol, substituted or unsubstituted dextran, substituted or unsubstituted cellulose, substituted or unsubstituted poly-(N-vinyl pyrrolidone) polyethylene glycol, substituted or unsubstituted propylene glycol homopolymers, substituted or unsubstituted polypropylene oxide/ethylene oxide co-polymers, substituted or unsubstituted polyoxyethylated polyols and/or substituted or unsubstituted polyvinyl alcohol.

In one embodiment, PS is divalent. In one embodiment, non-peptidic polymers suitable for use as moiety PS in Formula I include unsubstituted or substituted alkylene, unsubstituted or substituted heteroalkylene, unsubstituted or substituted cycloalkylene, unsubstituted or substituted heterocycloalkylene, unsubstituted or substituted arylene, and unsubstituted or substituted heteroarylene, which if substituted are in turn substituted with substituted or unsubstituted PEG, substituted or unsubstituted monomethoxy-polyethylene glycol, substituted or unsubstituted dextran, substituted or unsubstituted cellulose, substituted or unsubstituted poly-(N-vinyl pyrrolidone) polyethylene glycol, substituted or unsubstituted propylene glycol homopolymers, substituted or unsubstituted polypropylene oxide/ethylene oxide co-polymers, substituted or unsubstituted polyoxyethylated polyols and substituted or unsubstituted polyvinyl alcohol.

Additional suitable water-soluble polymers or mixtures thereof include N-linked or O-linked carbohydrates, sugars (e.g. various polysaccharides such as chitosan, xanthan gum, cellulose and its derivatives, acacia gum, karaya gum, guar gum, carrageenan, and agarose) and phosphates.

Polyethylene glycol (including the forms of PEG that have been used to derivatize proteins) include mono-(C₁-C₁₈ alkyl)-, alkoxy-, or aryloxy-polyethylene glycol and monomethoxy-polyethylene glycol.

A non-peptidic spacer PS can be non-immunogenic, biologically inert and hydrophilic. In addition, the preferred linkers are capable of conveying desirable properties to the biologically active polypeptidic groups. Such properties include reduced immunogenicity, increased solubility, and/or reduced clearance rate from the body without significantly reducing the affinity of P₁ and/or P₂ to their respective receptors, or without significantly reducing in vivo potency.

Without wishing to be bound by any theory, it is believed that the water-soluble moiety PS will function to sufficiently separate the peptide hormones in the LDDHC to improve, restore or maintain a biological activity (or potency or efficacy) of either or both peptides at least comparable to that of either or both unconjugated parent peptides or superior to that of either or both peptides in a reference conjugate without the PS as a spacer (as disclosed herein) or to the reference conjugate without the PS as a spacer but with the PS attached at a pendant amino acid side chain of the reference conjugate's backbone (as disclosed herein).

Without further wishing to be bound by any theory, it is believed that the water-soluble moiety PS will function as a half-life extending moiety. “Half-life extending moiety” refers to a moiety which increases the duration of biological activity of a conjugate to which it is bound. Measurement of duration of biological activity can be conducted by any suitable method or assay known in the art. Specifically, this is a moiety that prevents or mitigates in vivo degradation by proteolysis or other activity-diminishing chemical modification, increases in vivo half-life or other pharmacokinetic properties such as but not limited to increasing the rate of absorption, reduces toxicity, reduces immunogenicity, improves solubility, increases biological activity and/or target selectivity of the fusion protein with respect to a target of interest, and/or increases manufacturability, compared to an unconjugated form of the peptides included in a compound as set forth for Formula (I). In one embodiment, the half-life extending moiety is one that is pharmaceutically acceptable. The half-life extending moiety should be selected such that the LDDHC achieves a sufficient hydrodynamic size to reduce clearance by renal filtration in vivo. For example, a half-life extending moiety can be selected that is a polymeric macromolecule, which is substantially straight chain, branched-chain, or dendritic in form. Alternatively, a half-life extending moiety can be selected such that, in vivo, the inventive composition of matter will bind to a plasma protein to form a complex, such that the complex thus formed avoids or reduces substantial renal clearance. Exemplary half-life extending moieties that can be used include a polyalkylene glycol compound, such as a PEG or a polypropylene glycol. Other appropriate polyalkylene glycol compounds include charged or neutral polymers of the following types: dextran, colominic acids or other carbohydrate based polymers, polymers of amino acids, and biotin derivatives.

Surprisingly, the LDDHC can also have a reduced tendency to form undesirable vacuoles in kidney in vivo as disclosed herein.

Other examples of the half-life extending moieties useful as a PS in the compounds described herein include a copolymer of ethylene glycol, a copolymer of propylene glycol, a carboxymethylcellulose, a polyvinyl pyrrolidone, a poly-1,3-dioxolane, a poly-1,3,6-trioxane, an ethylene/maleic anhydride copolymer, a polyaminoacid (e.g., polylysine or polyornithine), a dextran n-vinyl pyrrolidone, a poly n-vinyl pyrrolidone, a propylene glycol homopolymer, a propylene oxide polymer, an ethylene oxide polymer, a polyoxyethylated polyol, a polyvinyl alcohol, a linear or branched glycosylated chain, a polyacetal, a long chain fatty acid, a long chain hydrophobic aliphatic group, where the moieties are 30 to 80 kDa, while being water soluble. In being conjugated, the half-life extending moiety, as described herein, is covalently bound directly to an amino acid residue of a compound described herein, or optionally, to a peptidyl or non-peptidyl linker (including but not limited to aromatic or aryl linkers and linkers described herein) that is covalently bound to an amino acid residue of the peptide compound.

In one embodiment, the water soluble polymer spacer of Formula I is a polyethylene glycol (PEG) molecule with molecular weight in the range 30-80 kDa. In one embodiment, the size range is 30-80 kDa, 30-70 kDa, 30-60 kDa, 35-60 kDa or 35-50 kDa. In one embodiment, the PEG water soluble spacer is 30 kDa, 35 kDa, 40 kDa, 45 kDa, 50 kDa, 60 kDa, 70 kDa, and even 80 kDa.

In one embodiment, the polydispersity of water soluble spacers is less than about 1.2, as measured by methods known in the art. In one embodiment, the polydispersity lies in the range of about 1.01 to about 1.20. In one embodiment, the polydispersity is about 1.01, 1.02, 1.03, 1.04, 1.05, 1.06, 1.07, 1.08, 1.09, 1.10, 1.11, 1.12, 1.13, 1.14, 1.15, 1.16, 1.17, 1.18, 1.19 or even 1.20.

Techniques for the preparation of activated PEG and its conjugation to biologically active peptides are well known in the art. (e.g., see U.S. Pat. Nos. 5,643,575, 5,919,455, 5,932,462, and 5,990,237; Thompson et al., PEGylation of polypeptides, EP 0575545 B1; Petit, Site specific protein modification, U.S. Pat. Nos. 6,451,986, and 6,548,644; S. Herman et al., Poly(ethylene glycol) with reactive endgroups: I. Modification of proteins, J. Bioactive Compatible Polymers, 10:145-187 (1995); Y. Lu et al., Pegylated peptides III: Solid-phase synthesis with PEGylating reagents of varying molecular weight: synthesis of multiply PEGylated peptides, Reactive Polymers, 22:221-229 (1994); A. M. Felix et al., PEGylated Peptides IV: Enhanced biological activity of site-directed PEGylated GRF analogs, Int. J. Peptide Protein Res., 46:253-264 (1995); A. M. Felix, Site-specific polyethylene glycol)ylation of peptides, ACS Symposium Series 680(poly(ethylene glycol)): 218-238 (1997); Y. Ikeda et al., Polyethylene glycol derivatives, their modified peptides, methods for producing them and use of the modified peptides, EP 0473084 B1; G. E. Means et al., Selected techniques for the modification of protein side chains, in: CHEMICAL MODIFICATION OF PROTEINS, Holden Day, Inc., 219 (1971)).

Activated PEG, such as PEG-aldehydes or PEG-aldehyde hydrates, can be chemically synthesized by known means or obtained from commercial sources, e.g., Shearwater Polymers, (Huntsville, Ala.) or Enzon, Inc. (Piscataway, N.J.).

An example of an activated PEG useful in the synthesis of LDDHCs described herein is a PEG-aldehyde compound (e.g., a methoxy PEG-aldehyde), such as PEG-propionaldehyde, which is commercially available from Shearwater Polymers (Huntsville, Ala.). PEG-propionaldehyde is represented by the formula PEG-CH₂CH₂CHO. See, e.g., U.S. Pat. No. 5,252,714. Also included within the meaning of “PEG aldehyde compound” are PEG aldehyde hydrates, e.g., PEG acetaldehyde hydrate and PEG bis aldehyde hydrate, which latter yields a bifunctionally activated structure. (See., e.g., Bentley et al., Poly(ethylene glycol) aldehyde hydrates and related polymers and applications in modifying amines, U.S. Pat. No. 5,990,237) (See., e.g., Bentley et al., Poly(ethylene glycol) aldehyde hydrates and related polymers and applications in modifying amines, U.S. Pat. No. 5,990,237). An activated multi-branched PEG-aldehyde compound can be used to afford PEG derivatives including multiple arms to give divalent, trivalent, tetravalent, octavalent constructs and the like, as known in the art.

In one embodiment, on being incorporated into a LDDHC, the PEG can be covalently bound by reductive amination directly to at least one solvent-exposed free amine moiety of an amino acid residue of a polypeptide component described herein. In one embodiment, the LDDHC of Formula (I) is bonded, through L₁ and L₂, to a PEG at one or more primary or secondary amines on the recombinant fusion protein, or to two PEG groups at a single primary amine site on the fusion protein. For example, this can occur when the reductive amination reaction involves the presence of excess PEG-aldehyde compound. In one embodiment, when PEGylated product with a single PEG per PEGylation product molecule is desired, PEGylation using secondary amines can be conducted wherein only one PEG group per molecule will be transferred in the reductive animation reaction.

Amino acid residues that can provide a primary amine moiety include residues of lysine, homolysine, ornithine, α,β-diaminopropionic acid (Dap), α,β-diaminopropionoic acid (Dpr), and α,γ-diaminobutyric acid (Dab), aminobutyric acid (Abu), and α-amino-isobutyric acid (Aib). The polypeptide N-terminus also provides a useful α-amino group for PEGylation. Amino acid residues that can provide a secondary amine moiety include ε-N-alkyl lysine, α-N-alkyl lysine, δ-N-alkyl ornithine, α-N-alkyl ornithine, or an N-terminal proline, where the alkyl is C₁ to C₆ alkyl.

Another useful activated PEG for synthesis of compounds described herein is a PEG-maleimide compound, such as a methoxy PEG-maleimide. See e.g., U.S. Pat. No. 6,602,498; C. Delgado et al., Crit. Rev. Therap. Drug Carrier Systems 9:249-304 (1992); S. Zalipsky et al., in: Poly(ethylene glycol) chemistry: Biotechnical and biomedical applications (J. M. Harris, Editor, Plenum Press: New York, 347-370 (1992); S. Herman et al., J. Bioactive Compatible Polymers 10:145-187 (1995); U.S. Pat. No. 4,847,325; U.S. Pat. No. 5,166,322; EP 0469074 B1; EP 0668353 A1; EP 0668354 A1; U.S. Pat. No. 5,206,344; R. J. Goodson and N. V. Katre, Biotechnolog, 8:343-346 (1990).

A poly(ethylene glycol) vinyl sulfone is another useful activated PEG for generating the PEG-conjugated compounds described herein by conjugation at thiolated amino acid residues, e.g., at C residues. See e.g., M. Morpurgo et al., Bioconj. Chem. 7:363-368 (1996); U.S. Pat. Nos. 5,446,090; 5,739,208; 5,900,461; 6,610,281 and 6,894,025; WO 95/13312 A1.

Another activated form of PEG that is useful in synthesis of compounds described herein is a PEG-N-hydroxysuccinimide ester compound, for example, methoxy PEG-N-hydroxysuccinimidyl (NHS) ester.

Heterobifunctionally activated forms of PEG are also useful. See e.g., U.S. Pat. No. 6,552,170.

Also useful are thiol-activated PEG compounds including a diol-activated PEG compound, a PEG-hydrazide compound, a PEG-oxyamine compound, or a PEG-bromoacetyl compound. See, e.g., S. Herman, J. Bioactive and Compatible Polymers 10:145-187 (1995); S. Zalipsky, Advanced Drug Delivery Reviews 16:157-182 (1995); R. Greenwald et al., Critical Reviews in Therapeutic Drug Carrier Systems 17:101-161 (2000).

The smallest practical size of PEG is about 500 Daltons (Da), below which PEG becomes toxic. Above about 500 Da, any molecular mass for a PEG can be used as practically desired, e.g., from about 1,000 Daltons (Da) to 100,000 Da (n is 20 to 2300). The number of PEG monomers (n) is approximated from the average molecular mass using a MW=44 Da for each monomer. It is preferred that the combined molecular mass of PEG on an activated linker is suitable for pharmaceutical use.

In still other embodiments, component peptides of compounds described herein are reacted by known chemical techniques with an activated multi-branched PEG compound (PEG derivatives comprising multiple arms to give divalent, trivalent, tetravalent, octavalent constructs), such as pentaerythritol tetra-polyethyleneglycol ether. Functionalization and activated derivatives, such as N-succinimidyloxycarbonyl)propyl, p-nitrophenyloxycarbonyl, (—CO₂-p-C₆H₄NO₂), 3-(N-maleimido)propanamido, 2-sulfanylethyl, and 3-aminopropyl.

### Exemplary Compounds

### Nomenclature

Regarding nomenclature of compounds (e.g., LDDHCs and other compounds) described herein, square brackets (“[ ]”) indicate separate fragments and crosshatch (“#”) indicates linking positions. As is customary in the art, square brackets also indicate peptide substitutions, and the use of this nomenclature will be clear from context. For example, in Cmpd 35, with nomenclature “[Cmpd 3-Lys(Cys(Mal-PEG35K-Mal#)), amide] [Cmpd 3-Lys(Cys(#)), amide]” the pendant ε-amino group of a lysine attached at the C-terminal of Cmpd 3 is in turn bonded to cysteine via an amide linkage, which cysteine is in turn bonded to maleimide, which moiety in turn is bonded to a PEG moiety. The PEG moiety is in turn bonded to a maleimide, which in turn is bonded to a cysteine, which in turn is bonded to the side chain ε-amino group of a lysine at the C-terminal of Cmpd 3. The chemical nature of the bond indicated by the “#” symbol will be clear to one of skill in the art from context. For example, for Cmpd 35, the “#” symbol indicates linkage through a thioether bond between Cys and maleimide. Further regarding nomenclature including conventions employed in the tables provided herein, absent indication to the contrary a PEG moiety is attached to the backbone of the peptide. For example, Cmpd 67 is the result of the conjugation of mPEG40 KD to the N-terminal nitrogen of Cmpd 49. Similarly, Cmpd 68 is the result of conjugation of mPEG40 KD to the N-terminal nitrogen of Cmpd 50. Standard single letter abbreviations for amino acids can be used, as can standard three-letter abbreviations. For example, Cmpd 72 is an analog of Cmpd 54 wherein the pendant amine functionality of lysine 26 (i.e., Nε of K²⁶) is conjugated with a PEG40 KD moiety. Exemplary compounds are provided in Table 1b below. The term “PEGXXKD” refers to a polyethylene glycol moiety having nominal molecular weight of “XX” kDa; e.g., PEG40 KD refers to a polyethylene glycol having nominal molecular weight 40 kDa. The term “mPEG” refers, as customary in the art, to methoxyl-PEG.

Further exemplary of the use of crosshatches to indicate linking position is Cmpd 14 with formula “Cmpd 3-Lys(Cys(#)][#Mal-PEG40 KD-[des-Lys¹]Cmpd 4” having the structure following, wherein “n” is of sufficient size to afford a PEG40 KD moiety; e.g., n is about 900 for PEG40 KD.

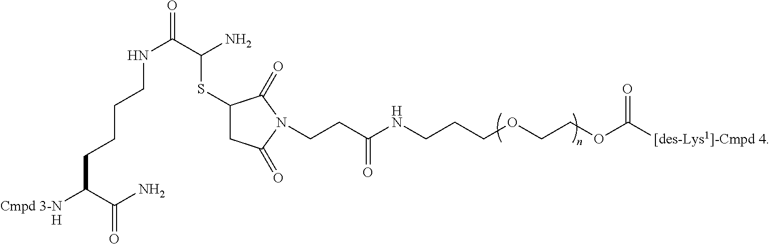

In one embodiment, compounds contemplated herein include an exendin as P₁ and a P₂ selected from an exendin, an amylin, pramlintide, davalintide, or analogs or derivatives thereof. P₁, P₂ and PS are as described herein. Representative compounds include compounds with the structure exendin-L₁-PS-L₂-amylin, exendin-L₁-PS-L₂-pramlintide, and exendin-L₁-PS-L₂-davalintide. Further representative compounds include an amylin or analog or derivative thereof as P₁, including e.g., amylin-L₁-PS-L₂-amylin, amylin-L₁-PS-L₂-pramlintide, and amylin-L₁-PS-L₂-davalintide. Yet further representative compounds include a davalintide or analog or derivative thereof as P 1, including e.g., davalintide-L₁-PS-L₂-amylin or davalintide-L₁-PS-L₂-davalintide. Exemplary peptides, peptide derivatives, short peptide conjugates and reagents described herein are provided in Table 1a following.

TABLE 1a
Exemplary peptides, peptide derivatives, short peptide conjugates
and reagents

| Cmpd | Com pound Description |
|---|---|
| 1 | Exendin-4: HGEGTFTSDLSKQMEEEAVRLFIEWLKNGGPSSGAPPPS-NH₂ |
| | (SEQ ID NO: 6) |
| 2 | Exendin-4 (1-28): |
| | HGEGTFTSDLSKQMEEEAVRLFIEWLKN (SEQ ID NO: 68) |
| 3 | [Leu¹⁴]Exendin-4: |
| | HGEGTFTSDLSKQLEEEAVRLFIEWLKNGGPSSGAPPPS-NH₂ (SEQ ID NO: 7) |
| 4 | davalintide: cyclo²⁻⁷ KCNTATCVLGRLSQELHRLQTYPRTNTGSNTY-NH₂ |
| | (SEQ ID NO: 34). |
| 5 | [Lys¹-N_{ε}-mPEG40KD]-Cmpd 4 (SEQ ID NO: 34) |
| 6 | des Lys¹[(N_{α}-mPEG40KD)-Cys²]-Cmpd 4 (SEQ ID NO: 69) |
| 7 | Cmpd 3-[N_{ε}-Cys(mPEG 40KD)]Lys: |
| | HGEGTFTSDLSKQLEEEAVRLFIEWLKNGGPSSGAPPPSK(N_{ε}-C(mPEG40KD))- |
| | NH₂ (SEQ ID NO: 70) |
| 15 | HGEGTFTSDLSKQMEEEAVRLFIEWLKN-(bAla)-(bAla)- |
| | KCNTATCVLGRLSQELHRLQTYPRTNTGSNTY-NH₂ (SEQ ID NO: 71) |
| 16 | HGEGTFTSDLSKQLEEEAVRLFIEWLKQGGPSKEIISGGGKCNTATCVLGRLSQE |
| | LHRLQTYPRTNTGSNTY-NH₂ (SEQ ID NO: 72) |
| 17 | MeO-PEG 20K |
| 34 | HGEGTFTSDLSKQLEEEAVRLFIEWLKQGGPSKEIIS-OH (SEQ ID NO: 73) |
| 49 | KCNTATCATQRLANFLVRSSNNLGPVLPPTNVGSNTY-NH₂ (SEQ ID NO: 28) |
| 50 | ([des-Lys¹]-Cmpd 49): CNTATCATQRLANFLVRSSNNLGPVLPPTNVGSNTY-NH₂ |
| | (SEQ ID NO: 75) |
| 51 | KCNTATCATQRLANFLVRSSKNLGPVLPPTNVGSNTY-NH₂ (SEQ ID NO: 76) |
| 52 | ([des-Lys¹]-Cmpd 51): CNTATCATQRLANFLVRSSKNLGPVLPPTNVGSNTY-NH₂ |
| | (SEQ ID NO: 77) |
| 53 | KCNTATCATQRLANFLVRSSNNLGPKLPPTNVGSNTY-NH₂ (SEQ ID NO: 78) |
| 54 | ([des-Lys¹]-Cmpd 53): CNTATCATQRLANFLVRSSNNLGPKLPPTNVGSNTY-NH₂ |
| | (SEQ ID NO: 79) |
| 55 | KCNTATCATQRLANFLVRSSNNLGPVLPPTKVGSNTY-NH₂ (SEQ ID NO: 80) |
| 56 | ([des-Lys¹]-Cmpd 55): CNTATCATQRLANFLVRSSNNLGPVLPPTKVGSNTY-NH₂ |
| | (SEQ ID NO: 81) |
| 57 | KCNTATCATQRLANFLVHSSNNFGPILPPTNVGSNTY-NH₂ (SEQ ID NO: 82) |
| 58 | ([des-Lys¹]-Cmpd 57): CNTATCATQRLANFLVHSSNNFGPILPPTNVGSNTY-NH₂ |
| | (SEQ ID NO: 83) |
| 59 | CNTATCATQRLANFLVHSSKNFGPILPPTNVGSNTY-NH₂ (SEQ ID NO: 84) |
| 60 | CNTATCATQRLANFLVHSSNNFGPKLPPTNVGSNTY-NH₂ (SEQ ID NO: 85) |
| 61 | CNTATCATQRLANFLVHSSNNFGPILPPTKVGSNTY-NH₂ (SEQ ID NO: 86) |
| 62 | CNTATCATQRLANFLVHSSNNFKPILPPTNVGSNTY-NH₂ (SEQ ID NO: 87) |
| 63 | CNTATCATQRLANFLVHSSNNFGKILPPTNVGSNTY-NH₂ (SEQ ID NO: 88) |
| 64 | CNTATCATQRLANFLVHSSNNFGPIKPPTNVGSNTY-NH₂ (SEQ ID NO: 89) |
| 65 | CNTATCATQRLANFLVHSSNNFGPILKPTNVGSNTY-NH₂ (SEQ ID NO: 90) |
| 66 | CNTATCATQRLANFLVHSSNNFGPILPKTNVGSNTY-NH₂ (SEQ ID NO: 91) |

TABLE 1b
Exemplary pegylated peptides and peptide derivatives

| Cmpd | Compound Description |
|---|---|
| 67 | mPEG40KD-Cmpd 49 (SEQ ID NO: 92) |
| 68 | mPEG40KD-Cmpd 50 (SEQ ID NO: 93) |
| 69 | [K²¹(mPEG40KD)]-Cmpd 51 (SEQ ID NO: 94) |
| 70 | [K²¹(mPEG40KD)]-Cmpd 52 (SEQ ID NO: 95) |
| 71 | [K²⁶(mPEG40KD)]-Cmpd 53 (SEQ ID NO: 96) |
| 72 | [K²⁶(mPEG40KD)]-Cmpd 54 (SEQ ID NO: 97) |
| 73 | [K³¹(mPEG40KD)]-Cmpd 55 (SEQ ID NO: 98) |
| 74 | [K³¹(mPEG40KD)]-Cmpd 56 (SEQ ID NO: 99) |
| 75 | [K²⁶(Y-shaped-mPEG40KD)]-Cmpd 53 (SEQ ID NO: 100) |
| 76 | [K²¹(mPEG40KD)]-Cmpd 59 (SEQ ID NO: 101) |
| 77 | [K²⁶(mPEG40KD)]-Cmpd 60 (SEQ ID NO: 102) |
| 78 | [K³¹(mPEG40KD)]-Cmpd 61 (SEQ ID NO: 103) |
| 79 | [K²⁶(Y-shaped-mPEG40KD)]-Cmpd 60 (SEQ ID NO: 104) |
| 80 | [K²⁴(mPEG40KD)]-Cmpd 62 (SEQ ID NO: 105) |
| 81 | [K²⁵(mPEG40KD)]-Cmpd 63 (SEQ ID NO: 106) |
| 82 | [K²⁷(mPEG40KD)]-Cmpd 64 (SEQ ID NO: 107) |
| 83 | [K²⁸(mPEG40KD)]-Cmpd 65 (SEQ ID NO: 108) |
| 84 | [K²⁹(mPEG40KD)]-Cmpd 66 (SEQ ID NO: 109) |
| 85 | [K²²(mPEG40KD)]-Cmpd 58 (SEQ ID NO: 110) |
| 86 | [K²³(mPEG40KD)]-Cmpd 58 (SEQ ID NO: 111) |
| 87 | [K³⁰(mPEG40KD)]-Cmpd 58 (SEQ ID NO: 112) |
| 88 | [K²⁶(mPEG40KD)]-Cmpd 58 (SEQ ID NO: 113) |
| 89 | [K¹⁷(mPEG40KD)]-Cmpd 58 (SEQ ID NO: 114) |
| 90 | [K¹⁸(mPEG40KD)]-Cmpd 58 (SEQ ID NO: 115) |
| 91 | [K²⁰(mPEG40KD)]-Cmpd 58 (SEQ ID NO: 116) |
| 92 | [K³²(mPEG40KD)]-Cmpd 58 (SEQ ID NO: 117) |
| 93 | [K³³(mPEG40KD)]-Cmpd 58 (SEQ ID NO. 118) |
| 94 | [K³⁴(mPEG40KD)]-Cmpd 58 (SEQ ID NO: 119) |
| 95 | [K³⁵(mPEG40KD)]-Cmpd 58 (SEQ ID NO: 120) |
| 96 | [K³⁶(mPEG40KD)]-Cmpd 58 (SEQ ID NO: 121) |

A most preferred compound or polypeptide conjugate is Cmpd 14.

Exemplary compounds (LDDHCs) useful for the compounds, compositions and methods described herein include compounds disclosed in Table 2 following.

TABLE 2
Exemplary LDDHCs

| Cmpd | Compound Description |
|---|---|
| 8 | Cmpd 2-GGG-Cmpd 4 hybrid (linear) (SEQ ID NO: 122) |
| 9 | Cmpd 3-GGG-Cmpd 4 hybrid (SEQ ID NO: 123) |
| 10 | Cmpd 2-GC(mPEG40KD)G-Cmpd 4 (hybrid (“T” 40KD PEG)) |
| | (SEQ ID NO: 124) |
| 11 | Cmpd 3-GC(mPEG40KD)G-Cmpd 4 (hybrid (“T” 40K PEG)) |
| | (SEQ ID NO: 74) |
| 12 | Cmpd 2-PEG 12KD-[des-Lys¹]-Cmpd 4 |
| 13 | [Cmpd 3-Lys(Cys(#)][#Mal-PEG12KD-[des-Lys¹]-Cmpd 4] |
| 14 | [Cmpd 3-Lys(Cys(#)][#Mal-PEG40KD-[des-Lys¹]-Cmpd 4] |
| 35 | [Cmpd 3-Lys(Cys(Mal-PEG35KD-Mal#)), amide] [Cmpd 3- |
| | Lys(Cys(#)), amide] |
| 45 | Cmpd 3-PEG 30KD-[des-Lys¹]-Cmpd 4 |
| 46 | Cmpd 3-PEG 60KD-[des-Lys¹]-Cmpd 4 |
| 47 | Cmpd 3-PEG 80KD-[des-Lys¹]-Cmpd 4 |
| 48 | [Cmpd 3-Lys-Cys(#)-Gly-acid][#Mal-PEG40K-des-Lys1- |
| | Cmpd 4] |

### Strategies for Compound Design

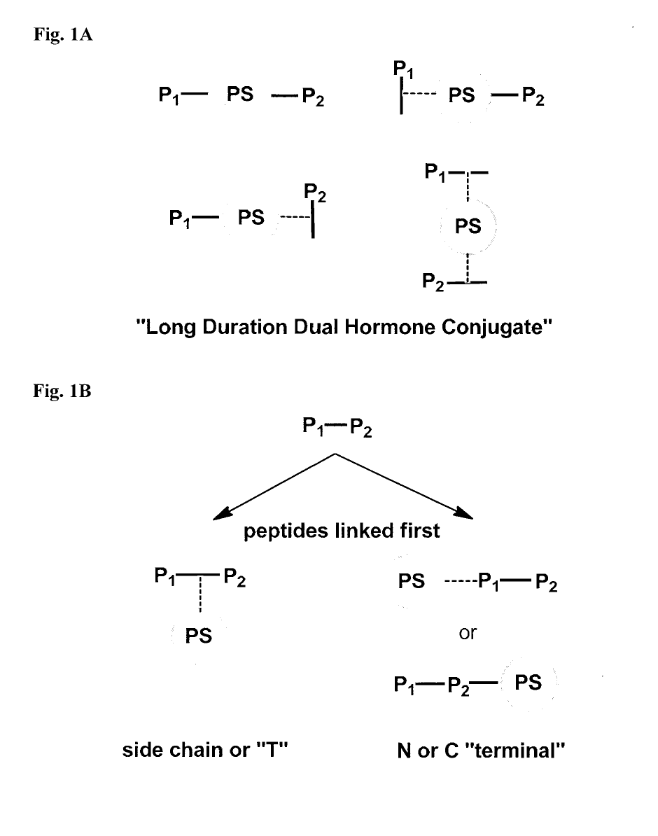

Compounds incorporating a plurality of peptide hormones in combination with one or more water-soluble polymeric spacers, including LDDHCs and other compounds described herein, may be described by a variety of general structural features which define the interconnection of the peptides, linkers and spacers contained therein. Without wishing to be bound by any theory, it is believed useful to conceive of such interconnections as depicted in the theoretical schemes shown in FIGS. 1A-B. In FIG. 1A, peptide hormone P₁ is bound at the C-terminal, optionally through a linker, to a water-soluble polymeric spacer. It is understood that in FIGS. 1A-B the symbol “PEG” is a surrogate for all suitable water-soluble polymeric spacers useful in the compounds described herein. Peptide hormone P₂ is bound at the N-terminal, optionally through a linker, to a water-soluble polymeric spacer. Conceptually, the process of forming a LDDHC can be envisaged as the coalescence of the water-soluble polymeric spacers to a single entity.

Complementary to the scheme depicted in FIG. 1A, in FIG. 1B peptide hormones P₁ and P₂ can be envisaged to be initially covalently bonded, optionally through a linker as described herein. Attachment of a water-soluble polymeric spacer can then occur via the linker (i.e., a so-called side chain or “T” motif), or at the C- or N-terminii of the linked P₁—P₂ moiety.

Biological results disclosed herein demonstrate that the activities of compounds described herein show an unexpected and surprising dependence on the properties (e.g., size) of the water-soluble polymeric spacer moiety of the compounds. Again without wishing to be bound by any theory, it is believed that a variety of explanations may be available for these observations. For example, when bound together, optionally through a linker of sufficiently small size, peptide hormones P₁ and P₂ may give rise to mutual interactions which are unfavorable for biological activity. Exemplary unfavorably interactions include, for example, steric, electrostatic, hydrophilic or hydrophobic interactions, as known in the art, which prevent proper activity (e.g., binding) at the biological receptor. Accordingly, as the size of the water-soluble polymeric spacer separating peptide hormones P₁ and P₂ increases, the mutual interaction between P₁ and P₂ decreases, and the individual biological activities of each peptide hormone with its biological receptor can then occur unimpeded.

### III. Exemplary Syntheses

General Methods of Polypeptide Synthesis.

The polypeptide components of the compounds described herein may be prepared using biological, chemical, and/or recombinant DNA techniques that are known in the art. Exemplary methods are described herein and in U.S. Pat. No. 6,872,700; WO 2007/139941; WO 2007/140284; WO 2008/082274; WO 2009/011544; and US Publication No. 2007/0238669, the disclosures of which are incorporated herein by reference in their entireties and for all purposes. Other methods for preparing the compounds are set forth herein and/or known in the art.

For example, the polypeptide components of the compounds described herein may be prepared using standard solid-phase peptide synthesis techniques, such as an automated or semi-automated peptide synthesizer. Typically, using such techniques, an alpha-N-carbamoyl protected amino acid and an amino acid attached to the growing peptide chain on a resin are coupled at room temperature in an inert solvent (e.g., dimethylformamide, N-methylpyrrolidinone, methylene chloride, and the like) in the presence of coupling agents (e.g., dicyclohexylcarbodiimide, 1-hydroxybenzo-triazole, and the like) in the presence of a base (e.g., diisopropylethylamine, and the like). The alpha-N-carbamoyl protecting group is removed from the resulting peptide-resin using a reagent (e.g., trifluoroacetic acid, piperidine, and the like) and the coupling reaction repeated with the next desired N-protected amino acid to be added to the peptide chain. Suitable N-protecting groups are well known in the art, such as t-butyloxycarbonyl (tBoc) fluorenylmethoxycarbonyl (Fmoc), and the like. The solvents, amino acid derivatives and 4-methylbenzhydryl-amine resin used in the peptide synthesizer may be purchased from a variety of commercial sources, including for example Applied Biosystems Inc. (Foster City, Calif.).

For chemical synthesis solid phase peptide synthesis can be used for the polypeptide conjugates, since in general solid phase synthesis is a straightforward approach with excellent scalability to commercial scale, and is generally compatible with relatively long polypeptide conjugates. Solid phase peptide synthesis may be carried out with an automatic peptide synthesizer (Model 430A, Applied Biosystems Inc., Foster City, Calif.) using the NMP/HOBt (Option 1) system and tBoc or Fmoc chemistry (See APPLIED BIOSYSTEMS USER′S MANUAL FOR THE ABI 430A PEPTIDE SYNTHESIZER, Version 1.3B Jul. 1, 1988, section 6, pp. 49-70, Applied Biosystems, Inc., Foster City, Calif.) with capping. Boc-peptide-resins may be cleaved with HF (−5° C. to 0° C., 1 hour). The peptide may be extracted from the resin with alternating water and acetic acid, and the filtrates lyophilized. The Fmoc-peptide resins may be cleaved according to standard methods (e.g., INTRODUCTION TO CLEAVAGE TECHNIQUES, Applied Biosystems, Inc., 1990, pp. 6-12). Peptides may also be assembled using an Advanced Chem Tech Synthesizer (Model MPS 350, Louisville, Ky.).

Compounds described herein can be assembled from component peptides (e.g., P₁, P₂), linkers (e.g., L₁, L₂) and water-soluble polymeric spacers by a variety of methods known in the art and described herein. For example, P₁-L₁-PS or P₁-L₁-PS-L₂ can be synthesized prior to chemical linkage with P2 to form P₁-L₁-PS-L₂-P₂.

Methods for Pegylation.

Covalent attachment of PEG can be conveniently achieved by a variety of methods available to one skilled in the synthetic chemical arts. For pegylation at backbone or side chain amine, PEG reagents are typically reacted under mild conditions to afford the pegylated compound. Optionally, additional steps including but not limited to reduction are employed. In a typical peptide-mPEG conjugation scheme, N-hydroxylsuccinimide (NHS) functionalized mPEG can be mixed with peptide having a free amine in a suitable solvent (e.g., dry DMF) under nitrogen in the presence of DIPEA (e.g., 3 equivalents per TFA counterion) for a suitable time (e.g., 24 hrs). The conjugate can be precipitated by the addition of a precipitation reagent (e.g., cold diethyl ether). The precipitate can be isolated by centrifugation and dissolved in water followed by lyophilization. Purification can be afforded by a variety of chromatographic procedures (e.g., MacroCap™ SP cation exchange column using gradient 0.5 M NaCl). Purity can be checked by SDS-PAGE. Mass spectrometry (e.g., MALDI) can be used to characterize the conjugate after dialysis against water.

PEG-SS (Succinimidyl Succinate).

PEG-SS reacts with amine groups under mild conditions to form the amide, as shown in Scheme 1. NHS functionalization provides amino reactive PEG derivatives that can react with primary amine groups at pH 7˜9 to form stable amide bonds. Reaction can be finished in 1 hour or even less time. Exemplary reactions follow in Schemes 1 and 2.

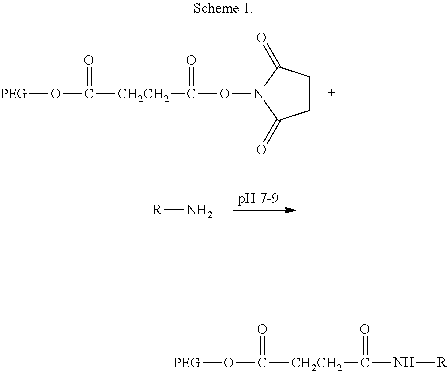

PEG-SG (Succinimidyl Glutarate).

Similarly, PEG-SG reacts with amine groups to form the corresponding amide, as shown in Scheme 2.

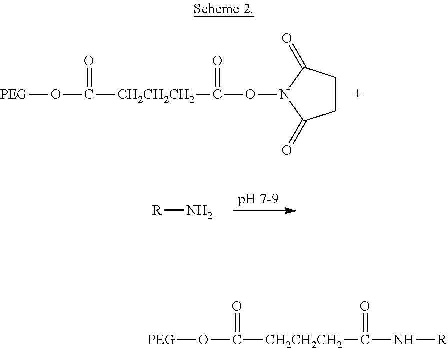

PEG-NPC (p-Nitrophenyl Carbonate).

PEG-NPC reacts with amine functionalities to form the relatively stable urethane functionality, as shown in Scheme 3.

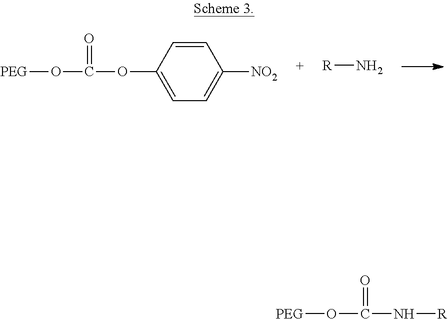

PEG-Isocyanate.

As shown in Scheme 4, PEG-isocyanate can react with amine to form the resultant relatively stable urethane linkage.

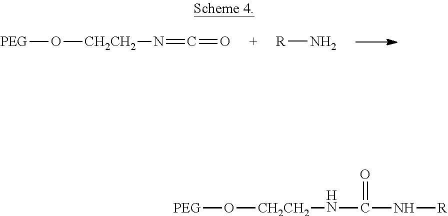

PEG-Aldehyde.

A variety of PEG-aldehyde reactions with amine can afford the imine, which can be further reduced to afford the pegylated amine. The reaction pH may be important for target selectivity. N-terminal amine pegylation may be at around pH 5. For example, reaction of mPEG-propionaldehyde with peptide amine, followed by reduction affords the compound depicted in Scheme 5 following.

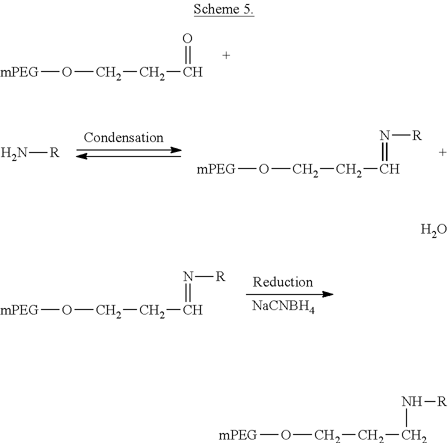

Similarly, condensation of mPEG-amide-propionaldehyde with amine and subsequent reduction can afford the compounds depicted in Scheme 6 following.

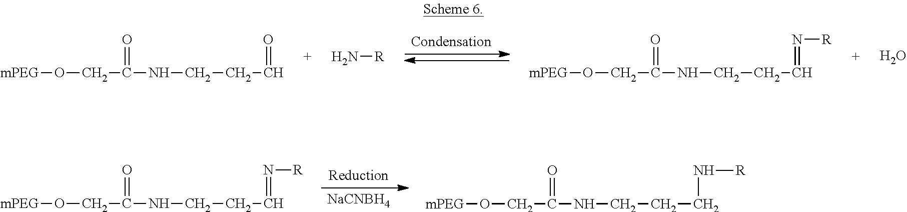

Reaction of mPEG-urethane-propionaldehyde with amine and subsequent reduction can afford the compounds depicted in Scheme 7 following.

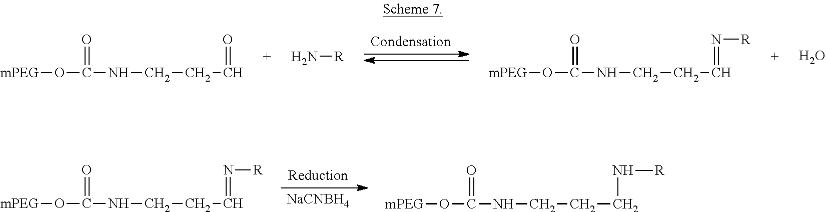

Furthermore, reaction of mPEG-butylaldehyde with amine and subsequent reduction can afford the compounds depicted in Scheme 8 following.

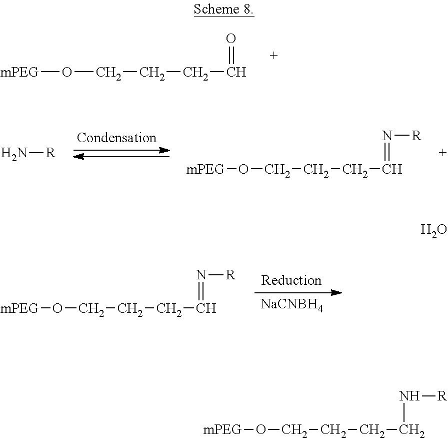

Thiol Pegylation: PEG-Maleimide.

Pegylation is conveniently achieved at free thiol groups by a variety of methods known in the art. For example, as shown in Scheme 9 following, PEG-maleimide pegylates thiols of the target compound in which the double bond of the maleimic ring breaks to connect with the thiol. The rate of reaction is pH dependent and best conditions are found around pH 8.

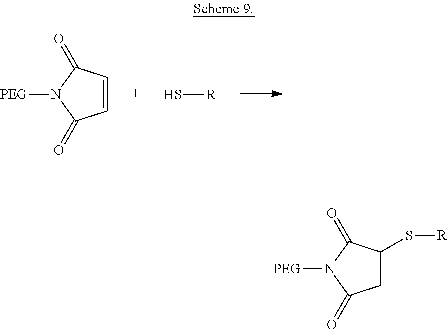

PEG-Vinylsulfone.

Additionally, as depicted in Scheme 10 following, PEG-vinylsulfone is useful for the pegylation of free thiol.

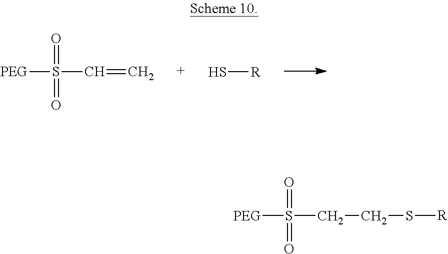

PEG-Orthopyridyl-Disulfide (OPSS).

Formation of disulfide linked PEG to a polypeptide is achieved by a variety of methods known in the art, including the reaction depicted in Scheme 11 following. In this type of linkage, the resulting PEG conjugate can be decoupled from the polypeptide by reduction with, for example but not limited to, borohydride, small molecule dithiol (e.g., dithioerythritol) and the like.

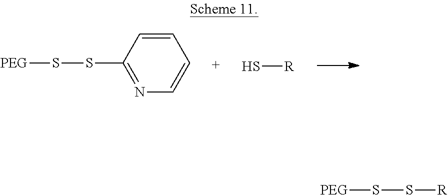

PEG-Iodoacetamide.

PEG-iodoacetamide pegylates thiols to form stable thioether bonds in mild basic media. This type of conjugation presents an interesting aspect in that by strong acid analysis the pegylated cysteine residue of the protein can give rise to carboxymethylcysteine which can be evaluated by a standard amino acid analysis (for example, amino acid sequencing), thus offering a method to verify the occurrence of the reaction. A typical reaction scheme is depicted in Scheme 12 following.

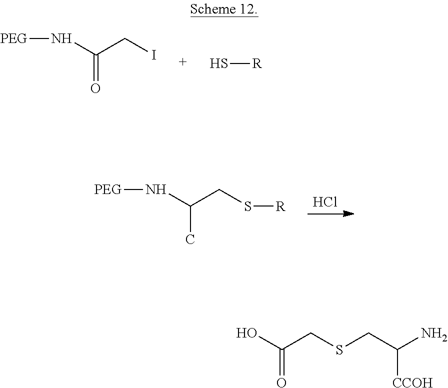

Transglutamination.

Transglutaminase (EC 2.3.1.13) catalyses the aminolysis of the γ-carboxamide group of the glutamine side chains of peptide and protein substrates. A typical reaction is disclosed in Scheme 13, wherein R—CONH₂ represents the acceptor, and R′—NH₂ is the donor alkylamine.

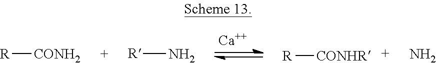

The reaction proceeds via an acyl-transfer mechanism in which the γ-carboxamide group acts as an acyl donor and suitably unbranched primary amines act as acyl acceptors. Accordingly, the reaction catalyzed by transglutaminase offers a method for selective introduction of functional groups into proteins under mild conditions. See e.g., Coussons et al., 1992, Biochem J. 283:803-806.

Modification of PEG at the carboxylic acid terminus to afford the alkylamine can proceed via a variety of routes, for example but not limited to the reaction shown in Scheme 14 following. In this scheme, alpha-carboxymethyl-ω-methoxypolyoxyethylene can be dissolved in N,N-dimethylformamide, to which 1-hydroxybenzotriazole (HOBO and dicyclohexyl carbodiimde (CDD) are added, and the reaction can proceed with time (e.g., 5-hrs) under a nitrogen atmosphere. To the reaction mixture, N-Boc-1,5-diaminopentane can be added, and the reaction can proceed with time, (e.g., 36-hrs) under nitrogen. The solvent can be removed, and the residue can be purified by column chromatography to afford the PEG-Boc compound. The Boc group can be removed with trifluoroacetic acid (TFA) to afford the PEG-alkylamine, suitable for reaction with transglutaminase. See Sato, 2002, Advanced Drug Delivery Reviews, 54:487-504.

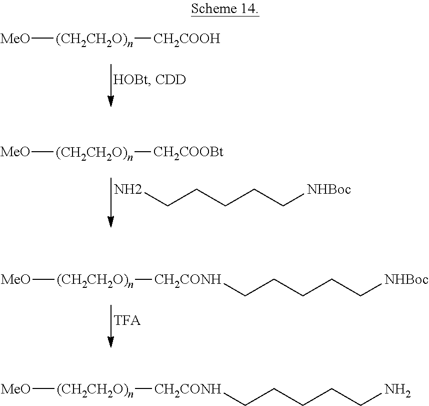

Bifunctional PEGylation Reagents.

A variety of bifunctional reagents useful for pegylation of the compounds described herein is available to the skilled artisan. Specific methods of pegylation are determined by the reactive groups on the pegylation reagent and the specific side chain and/or backbone conjugation desired. The pegylation reaction is as described herein, or as otherwise known in the art. In one embodiment, the pegylation reagent is a maleimide-PEG-carbonate NHS bifunctional reagent having nominal MW=40 kDa:

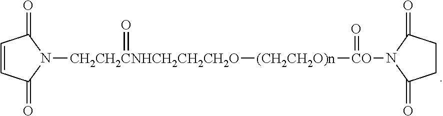

Additional Inert Conjugations.

A variety of additional water-soluble polymeric spacers suitable for use in the compounds described herein are available to the skilled artisan. In one embodiment, the water-soluble polymeric spacer is a hydroxyalkyl starch (HAS), preferably hydroxy ethyl starch (HES). See e.g., PCT Published Appl. Nos. WO 2005/014050, WO 2005/014655, WO 2005/092390, WO 2007/031266, WO 2005/092928 and WO 2005/092391, each of which is incorporated by reference herein and for all purposes. The terms “hasylation” and “hesylation” refer to conjugation of HAS and HES, respectively.

Purification of Compounds.

Purification of compounds described herein generally follows methods available to the skilled artisan. In a typical purification procedure, a crude peptide-PEG conjugate is initially purified via ion exchange chromatography, e.g., Macro Cap SP cation exchanger column. A typical purification procedure employs Buffer A (20 mM sodium acetate buffer, pH 5.0) and Buffer B (20 mM sodium acetate buffer, pH 5.0, 0.5 M sodium chloride) in a gradient elution program, e.g., 0-0% Buffer B (20 min), followed by 0-50% Buffer B (50 min), then 100% Buffer B (20 min). The flow rate is typically 3 mL/min. SDS polyacrylamide gel visualization of the collected fractions is conducted, followed by dialysis against water of the suitable fraction pool and lyophilization of the resultant. Analytical characterization typically employs MALDI mass spectroscopy.

### IV. Methods of Use

In one aspect, there is provided a method for the treatment of a disease or disorder. The method includes administering to a subject in need of treatment an effective amount of a compound or pharmaceutical composition described herein.

As used herein, a “subject” may include any mammal, including but not limited to rats, mice and humans. A “subject” also includes domestic animals (e.g., dogs, cats, horses), as well as other animals. As used herein, and as well-understood in the art, “treatment” is an approach for obtaining beneficial or desired results, including clinical results. “Treating,” “palliating,” or “ameliorating” a disease, disorder, or condition means that the extent, undesirable clinical manifestations of a condition, or both, of a disorder or a disease state are lessened and/or the time course of the progression is slowed (i.e., lengthened in time), as compared to not treating the disorder. For purposes of the methods disclosed herein, beneficial or desired clinical results include, but are not limited to, alleviation or amelioration of one or more symptoms, diminishment of extent of disorder, stabilized (i.e., not worsening) state of disorder, delay or slowing of disorder progression, amelioration or palliation of the disorder, and remission (whether partial or total), whether detectable or undetectable. “Treatment” can also mean prolonging survival as compared to expected survival if not receiving treatment. Further, treating does not necessarily occur by administration of one dose, but often occurs upon administration of a series of doses. Thus, a “therapeutically effective amount,” an amount sufficient to palliate, or an amount sufficient to treat a disease, disorder, or condition may be administered in one or more administrations.

In certain embodiments, the disease or disorder is diabetes, type 1 diabetes, type 2 diabetes, obesity, hypertension, atherosclerosis, dyslipidemia, congestive heart failure, stroke, hypercholesterolemia, cardiovascular disease, myocardial ischemia, myocardial reperfusion, an eating disorder, gestational diabetes, diabetic neuropathy, pulmonary hypertension or insufficient pancreatic beta cell mass. In certain embodiments, the subject is in need of treatment for regulating food intake, regulating body weight or regulating hematopoiesis.

In one embodiment, the disease or disorder is diabetes, type 1 diabetes, type 2 diabetes or gestational diabetes. In one embodiment, the disease or disorder is obesity. In one embodiment, the disease or disorder is hypertension, atherosclerosis, congestive heart failure, stroke, cardiovascular disease, myocardial ischemia, myocardial reperfusion or pulmonary hypertension. In one embodiment, the disease or disorder is dyslipidemia or hypercholesterolemia.

In one embodiment, the disease or disorder is diabetes, type I diabetes, type 2 diabetes, hypertension, atherosclerosis, dyslipidemia, congestive heart failure, stroke, hypercholesterolemia, cardiovascular disease, myocardial ischemia, myocardial reperfusion, gestational diabetes, diabetic neuropathy, pulmonary hypertension or insufficient pancreatic beta cell mass, and the subject in need thereof is overweight, obese, extremely obese or in need of body weight reduction. In one embodiment, the disease or disorder is diabetes, type 2 diabetes, diabetic neuropathy or insufficient pancreatic beta cell mass, and the subject in need thereof is overweight, obese, extremely obese or in need of body weight reduction.

Further to any method described herein, in one embodiment P₁ or the exendin analog of the compound of Formula (I) is [Leu¹⁴]exendin-4 or [Leu¹⁴,Lys⁴⁰]exendin-4. In one embodiment, P₂ or the amylin analog of the compound of Formula (I) is davalitide or [des-Lys¹]-davalintide. In one embodiment, PS of the compound of Formula (I) is a polyethylene glycol or a derivative thereof, and the polyethylene glycol or derivative thereof is linear. In one embodiment, the compound is Cmpd 14.

### V. Assays

The dual conjugate compounds described herein may be tested in a variety of receptor binding assays using binding assay methodologies generally known to those skilled in the art. Such assays include those described herein.

Food Intake.

A variety of food intake assays are available to one of skill in the art. For example, in the so-called “home cage model” of food intake, subjects (e.g., rats) are maintained in their home cage, and food intake along with total weight of the subject is measured following injection of test compound. In the so-called “feeding patterns model” of food intake assay, subjects (e.g., rats) are habituated to a feeding chamber and to injections prior to testing. After test compound administration, the subjects are immediately placed into the feeding chamber, and food intake is automatically determined as a function of time (e.g., 1-min intervals). For both tests, the food is standard chow or any of a variety of chows (e.g., high fat) known in the art. In the so-called “mouse food intake” assay, a test compound may be tested for appetite suppression, or for an effect on body weight gain in diet-induced obesity (DIO) mice. In a typical mouse food intake assay, female NIH/Swiss mice (8-24 weeks old) are group housed with a 12:12 hour light:dark cycle with lights on at 0600. Water and a standard pelleted mouse chow diet are available ad libitum, except as noted. Animals are fasted starting at approximately 1500 hrs, I day prior to experiment. The morning of the experiment, animals are divided into experimental groups. In a typical study, n=4 cages with 3 mice/cage. At time=0 min, all animals are given an intraperitoneal injection of vehicle or compound, typically in an amount ranging from about 10 nmol/kg to 75 nmol/kg, and immediately given a pre-weighed amount (10-15 g) of the standard chow. Food is removed and weighed at various times, typically 30, 60, and 120 minutes, to determine the amount of food consumed. See e.g., Morley et al., 1994, Am. J. Physiol. 267:R178-R184). Food intake is calculated by subtracting the weight of the food remaining at the e.g. 30, 60, 120, 180 and/or 240 minute time point, from the weight of the food provided initially at time=0. Significant treatment effects are identified by ANOVA (p<0.05). Where a significant difference exists, test means are compared to the control mean using Dunnett's test (Prism v. 2.01, GraphPad Software Inc., San Diego, Calif.). For any test described herein, administration of test compound can be by any means, including injection (e.g., subcutaneous, intraperitoneal, and the like), oral, or other methods of administration known in the art.

An additional exemplary assay involves the use of a diet-induced obese (DIO) mouse model for metabolic disease. Prior to the treatment period, male C57BL/6J, mice can be fed a high-fat diet (#DI2331, 58% of calories from fat; Research Diets, Inc.,) for 6 weeks beginning at 4 weeks of age. During the study; the mice can continue to eat their high-fat diet. Water can be provided ad libitum throughout the study. One group of similarly-aged non-obese mice can be fed a low-fat diet (#DI2329, 11% of calories from fat) for purposes of comparing metabolic parameters to DIO groups. DIO mice can be implanted with subcutaneous (SC) intrascapular osmotic pumps to deliver either vehicle (e.g., 50% dimethylsulfoxide (DMSO) in water) or a compound described herein. The pumps of the latter group can be set to deliver any amount, e.g. 1000 μg/kg/d of a compound for 7-28 days.

Blood Glucose.

Blood glucose can be measured by any of a variety of commercially available test kits, e.g., OneTouch® Ultra® (LifeScan, Inc. Milpitas, Calif.).

Calcitonin Adenylate Cyclase Assay (Functional Assay).

The calcitonin receptor mediated adenylate cyclase activation can be measured using an HTRF (Homogeneous Time-Resolved Fluorescence) cell-based cAMP assay kit from CisBio (Bedford, Mass.). This kit is a competitive immunoassay that uses cAMP labeled with the d2 acceptor fluorophore and an anti-cAMP monoclonal antibody labeled with donor Europium Cryptate. Increase in cAMP levels is registered as decrease in time-resolved fluorescence energy transfer between the donor and acceptor. Peptides can be serially diluted with buffer and transferred to, for example, a 384-well compound plate. C1a-HEK cells stably expressing the rat C1a calcitonin receptor can be detached from cell culture flasks and resuspended at 2×10⁶ cell/ml in stimulation buffer containing 500 μM IBMX, and d2 fluorophore at 1:40. Cells can be added to the compound plate at a density of 12,500 per well and incubated in the dark for 30 minutes at room temperature for receptor activation. Cells can be subsequently lysed by the addition of anti-cAMP Cryptate solution diluted with the kit conjugate/lysis buffer (1:40). After 1 to 24 hours incubation in the dark, the plate can be counted on a Tecan Ultra (Tecan Group, Ltd. Mannedorf, Switzerland) capable of measuring time-resolved fluorescence energy transfer.

GLP-1 Adenylate Cyclase Assay (Functional Assay).

The GLP-1 receptor mediated adenylate cyclase activation can be measured using an HTRF (Homogeneous Time-Resolved Fluorescence) cell based cAMP assay kit from CisBio. This kit is a competitive immunoassay that uses cAMP labeled with the d2 acceptor fluorophore and an anti-cAMP monoclonal antibody labeled with donor Europium Cryptate. Increase in cAMP levels is registered as decrease in time-resolved fluorescence energy transfer between the donor and acceptor. Peptides can be serially diluted with buffer and transferred to a 384-well compound plate. The rat thyroid C-cell line 6-23 cells endogenously expressing the rat GLP-1 receptor can be detached from cell culture flasks and resuspended at 2.5×106 cell/ml in stimulation buffer containing 500 μM IBMX, and d2 fluorophore at 1:40. Cells can be added to the compound plate at a density of 12,500 per well and incubated in the dark for 30 minutes at room temperature for receptor activation. Cells can be subsequently lysed by the addition of anti-cAMP Cryptate solution diluted with the kit conjugate/lysis buffer (1:40). After 1 to 24 hours incubation in the dark, the plate can be counted on a Tecan Ultra capable of measuring time-resolved fluorescence energy transfer.

Amylin Binding Assay.

Evaluation of the binding of exemplary compounds to amylin receptors can be carried out as follows in nucleus accumbens membranes prepared from rat brain. Male Sprague-Dawley® rats (200-250) grams can be sacrificed by decapitation. Brains can be removed and place in cold phosphate-buffered saline (PBS). From the ventral surface, cuts can be made rostral to the hypothalamus, bounded laterally by the olfactory tracts and extending at a 45° angle medially from these tracts. This basal forebrain tissue, containing the nucleus accumbens and surrounding regions, can be weighed and homogenized in ice cold 20 mM HEPES buffer (20 mM HEPES acid, pH adjusted to 7.4 with NaOH at 23° C.). Membranes can be washed three times in fresh buffer by centrifugation for 15 minutes at 48,000×G. The final membrane pellet can be resuspended in 20 mM HEPES buffer containing 0.2 mM phenylmethylsulfonyl fluoride (PMSF).

To measure ¹²⁵I-amylin binding (see e.g., Beaumont K et al., 1995, Can J Physiol Pharmacol. 73(7): 1025-9), membranes from 4 mg original wet weight of tissue can be incubated with ¹²⁵I-amylin at 12-16 μM in 20 mM HEPES buffer containing 0.5 mg/ml bacitracin, 0.5 mg/ml bovine serum albumin, and 0.2 mM PMSF. Solutions can be incubated for 60 minutes at 2° C. Incubations can be terminated by filtration through GF/B glass fiber filters (Whatman Inc., Clifton, N.J.) that are presoaked for 4 hours in 0.3% poylethyleneimine in order to reduce nonspecific binding of radiolabeled peptides. Filters can be washed immediately before filtration with 5 ml cold PBS, and immediately after filtration with 15 ml cold PBS. Filters can be removed and radioactivity assessed in a gamma-counter at a counting efficiency of e.g., 77%. Competition curves can be generated by measuring binding in the presence of 10-12 to 10-6 M unlabeled test compound and can be analyzed by nonlinear regression using a 4-parameter logistic equation (Inplot program; GraphPAD Software, San Diego). In another amylin receptor binding assay, ¹²⁵I-amylin (rat) is displaced from human amylin receptor 3 (AMY3) ectopically expressed in a cell line, e.g., a Codex ACTOne™ cell line. This cell line can be generated using ACTOne™ HEK293-CNG-hCalcR cell line (CB-80200-258) stably expressing human RAMP3 (NCBI protein database CAA04474) to produce the human AMY3 receptor.

CGRP Receptor Binding Assay.

Evaluation of the binding of compounds described herein to CGRP receptors can be essentially as described for amylin except using membranes prepared from SK-N-MC cells, known to express CGRP receptors. See e.g., Muff, R et al., Ann NY Acad. Sci. 1992, 657:106-16. Binding assays can be performed as described for amylin except using 13,500 cpm ¹²⁵I-hCGRP/well or 21.7 pM/well (Amersham).

Adrenomedullin Binding Assay.

Binding to the adrenomedullin receptor can be investigated using HUVECs that contain the adrenomedullin receptor (Kato Jet. al., Eur J. Pharmacol. 1995, 289:383-5) using the Perkin Elmer AlphaScreen™ assay for cyclic AMP using an optimum of 25-30,000 cells per well. Elevation of cAMP levels is not large for HUVEC compared to CRO cells. As such, CRO cells may be chosen as a negative control since they do not express the adrenomedullin receptor if desired.

Calcitonin Receptor Binding Assay.

Binding to the calcitonin receptor may be investigated using CRO cells or T47Dcells, which also express the calcitonin receptor. See e.g., Muff R. et. al, Id.; Kuestner R E. et. al. Mol. Pharmacol. 1994, 46:246-55.

GLP-1 Receptor Binding Assay.

GLP-1 receptor binding activity and affinity may be measured using a binding displacement assay in which the receptor source is RINm5F cell membranes, and the ligand is [¹²⁵I]GLP-1. Homogenized RINm5F cell membranes can be incubated in 20 mM HEPES buffer with 40,000 cpm e25I]GLP-1 tracer, and varying concentrations of test compound for 2 hours at 23° C. with constant mixing. Reaction mixtures can be filtered through glass filter pads presoaked with 0.3% PEI solution and rinsed with ice-cold phosphate buffered saline. Bound counts can be determined using a. scintillation counter. Binding affinities can be calculated using GraphPad Prism software (GraphPad Software, Inc., San Diego, Calif.).

Kidney Vacuolation Assay.

The kidney vacuolation assay is useful for measuring the filtering load by the glomerulus, as known in the art. The presence of vacuoles in the cytoplasm of epithelial cells lining the proximal convoluted tubules is observed by microscopic methods known in the art. Without wishing to be bound by any theory, it is believed that pegylated proteins are slowly filtered by the glomerulus, and are taken up via pinocytosis into lysosomes of the epithelial cells lining the proximal tubules. Lysosomal enzymes are able to process (e.g., hydrolyze) the protein component but not the PEG component. The hygroscopic nature of the PEG then causes fluid distention of the lysosomes, which are observed.

### VI. Pharmaceutical Compositions

In one aspect, there is provided a pharmaceutical composition which includes a peptide conjugate as described herein in combination with a pharmaceutically acceptable excipient.

A. Formulations

The compounds described herein can be prepared and administered in a wide variety of oral, parenteral, and topical dosage forms. Thus, the compounds described herein can be administered by injection (e.g. intravenously, intramuscularly, intracutaneously, subcutaneously, intraduodenally, or intraperitoneally). Also, the compounds described herein can be administered by inhalation, for example, intranasally. Additionally, the compounds can be administered transdermally. It is also envisioned that multiple routes of administration (e.g., intramuscular, oral, transdermal) can be used to administer the compounds. Accordingly, the present description also provides pharmaceutical compositions comprising a pharmaceutically acceptable carrier or excipient and one or more compounds described herein.

For preparing pharmaceutical compositions from the compounds described herein, pharmaceutically acceptable carriers can be either solid or liquid. Solid form preparations include powders, tablets, pills, capsules, cachets, suppositories, and dispersible granules. A solid carrier can be one or more substance that may also act as diluents, flavoring agents, binders, preservatives, tablet disintegrating agents, or an encapsulating material.

In powders, the carrier is a finely divided solid in a mixture with the finely divided active component. In tablets, the active component is mixed with the carrier having the necessary binding properties in suitable proportions and compacted in the shape and size desired.

The powders and tablets preferably contain from 5% to 70% of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

For preparing suppositories, a low melting wax, such as a mixture of fatty acid glycerides or cocoa butter, is first melted and the active component is dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions. For parenteral injection, liquid preparations can be formulated in solution in aqueous polyethylene glycol solution.

When parenteral application is needed or desired, particularly suitable admixtures for the compounds described herein are injectable, sterile solutions, preferably oily or aqueous solutions, as well as suspensions, emulsions, or implants, including suppositories. In particular, carriers for parenteral administration include aqueous solutions of dextrose, saline, pure water, ethanol, glycerol, propylene glycol, peanut oil, sesame oil, polyoxyethylene-block polymers, and the like. Ampoules are convenient unit dosages. The compounds described herein can also be incorporated into liposomes or administered via transdermal pumps or patches. Pharmaceutical admixtures suitable for use in the methods described herein include those described, for example, in PHARMACEUTICAL SCIENCES (17th Ed., Mack Pub. Co., Easton, Pa.) and WO 96/05309, the teachings of both of which are hereby incorporated by reference.

Aqueous solutions suitable for oral use can be prepared by dissolving the active component in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, and other well-known suspending agents.

Also included are solid form preparations that are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The quantity of active component in a unit dose preparation may be varied or adjusted, e.g., from 1 μg to 300 mg, 10 μg to 300 mg, 0.1 mg to 300 mg, 0.1 mg to 100 mg, 1.0 mg to 300 mg, 1.0 mg to 100 mg, more typically 0.1 mg to 10 mg, even more typically 0.1 mg to 5 mg, according to the particular application and the potency of the active component. In one embodiment, the daily dose is 1 μg to 1000 μg, e.g., 10 μg to 500 μg, 50 μg to 500 μg, or 100 μg to 400 μg. In one embodiment, the weekly dose is 1 μg to 7000 μg, e.g., 10 μg to 3500 μg, 50 μg to 3500 μg, or 100 μg to 2800 μg. In one embodiment, the weekly dose is 7 μg to 7000 μg, e.g., 70 μg to 3500 μg, 350 μg to 3500 μg, and 700 μg to 2800 μg. In one embodiment, the weekly dose is 100 μg to 350 m. In one embodiment, the lower end of the daily dosage range is 10 μg, 20 μg, 30 μg, 40 μg, 50 μg, 60 μg, 70 μg, 80 μg, 90 μg, 100 μg, 150 μg, 200 μg, 250 μg, 300 μg, 350 μg, or even greater. In one embodiment, the higher end of the daily dosage range is 1 mg, 2 mg, 3 mg, 4 mg, 5 mg, 6 mg, 7 mg, 8 mg, 9 mg, 10, mg, 20 mg, 30 mg, 60 mg, 100 mg, 200 mg, 300 mg or even greater. In one embodiment, the preferred daily dosage range is 0.1 mg to 60 mg, preferably 0.15 mg to 30 mg, more preferably 0.15 mg to 10 mg, even more preferably 0.15 to 0.3 mg. In one embodiment, the daily dose is 0.1 mg, 0.2 mg, 0.3 mg, 0.4 mg, 0.5 mg, 1.0 mg, 1.5 mg, 2.0 mg, 2.5 mg, 3.0 mg, 4.0 mg or even 5.0 mg. In one embodiment, the weekly dose is 0.1 mg, 0.2 mg, 0.3 mg, 0.4 mg, 0.5 mg, 1.0 mg, 1.5 mg, 2.0 mg, 3.0 mg, 4.0 mg, 5.0 mg, 6.0 mg, 7.0 mg, 8.0 mg, 9.0 mg, 10 mg, 20 mg, 30 mg, 35 mg or even greater. The composition can, if desired, also contain other compatible therapeutic agents.

Some compounds may have limited solubility in water and therefore may require a surfactant or other appropriate co-solvent in the composition. Such co-solvents include: Polysorbate 20, 60, and 80; Pluronic F-68, F-84, and P-103; cyclodextrin; and polyoxyl 35 castor oil. Such co-solvents are typically employed at a level between about 0.01% and about 2% by weight.

Viscosity greater than that of simple aqueous solutions may be desirable to decrease variability in dispensing the formulations, to decrease physical separation of components of a suspension or emulsion of formulation, and/or otherwise to improve the formulation. Such viscosity building agents include, for example, polyvinyl alcohol, polyvinyl pyrrolidone, methyl cellulose, hydroxy propyl methylcellulose, hydroxyethyl cellulose, carboxymethyl cellulose, hydroxy propyl cellulose, chondroitin sulfate and salts thereof, hyaluronic acid and salts thereof, and combinations of the foregoing. Such agents are typically employed at a level between about 0.01% and about 2% by weight.

The compositions described herein may additionally include components to provide sustained release and/or comfort. Such components include high molecular weight, anionic mucomimetic polymers, gelling polysaccharides, and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes.

B. Effective Dosages

Pharmaceutical compositions provided herein include compositions wherein the active ingredient is contained in a therapeutically effective amount, i.e., in an amount effective to achieve its intended purpose. The actual amount effective for a particular application will depend, inter alia, on the condition being treated. For example, when administered in methods to treat a neuropsychiatric disease or disorder, such compositions will contain an amount of active ingredient effective to achieve the desired result (e.g. relieving the symptoms of the neuropsychiatric disease or disorder).

The dosage and frequency (single or multiple doses) of compound administered can vary depending upon a variety of factors, including route of administration; size, age, sex, health, body weight, body mass index, and diet of the recipient; nature and extent of symptoms of the disease being treated (e.g., the neuropsychiatric disease responsive to amelioration); presence of other diseases or other health-related problems; kind of concurrent treatment; and complications from any disease or treatment regimen. Other therapeutic regimens or agents can be used in conjunction with the methods and compounds described herein.

For any compound described herein, the therapeutically effective amount can be initially determined from a variety of assays, including but not limited to cell culture assays and behavioral assays. Target concentrations will be those concentrations of active compound(s) that are capable of eliciting a biological response in cell culture assay, or eliciting a behavioral response.

Therapeutically effective amounts for use in humans may be determined from animal models. For example, a dose for humans can be formulated to achieve a concentration that has been found to be effective in animals. The dosage in humans can be adjusted by monitoring the underlying neuropsychiatric disease or disorder and adjusting the dosage upwards or downwards, as known in the art and/or as described herein.

Dosages may be varied depending upon the requirements of the patient and the compound being employed. The dose administered to a patient, in the context of the methods described herein, should be sufficient to affect a beneficial therapeutic response in the patient over time. The size of the dose also will be determined by the existence, nature, and extent of any adverse side effects. Generally, treatment is initiated with smaller dosages, which are less than the optimum dose of the compound. Thereafter, the dosage is increased by small increments until the optimum effect under circumstances is reached. In one embodiment, the dosage range is 0.001% to 10% w/v. In another embodiment, the dosage range is 0.1% to 5% w/v.

Dosage amounts and intervals can be adjusted individually to provide levels of the administered compound effective for the particular clinical indication being treated. This will provide a therapeutic regimen that is commensurate with the severity of the individual's disease state.

Utilizing the teachings provided herein, an effective prophylactic or therapeutic treatment regimen can be planned that does not cause substantial toxicity and yet is entirely effective to treat the clinical symptoms demonstrated by the particular patient. This planning should involve the careful choice of active compound by considering factors such as compound potency, relative bioavailability, patient body weight, presence and severity of adverse side effects, preferred mode of administration, and the toxicity profile of the selected agent.

C. Toxicity

The ratio between toxicity and therapeutic effect for a particular compound is its therapeutic index and can be expressed as the ratio between LD₅₀ (the amount of compound lethal in 50% of the population) and ED₅₀ (the amount of compound effective in 50% of the population). Compounds that exhibit high therapeutic indices are preferred. Therapeutic index data obtained from cell culture assays and/or animal studies can be used in formulating a range of dosages for use in humans. The dosage of such compounds preferably lies within a range of plasma concentrations that include the ED₅₀ with little or no toxicity. The dosage may vary within this range depending upon the dosage form employed and the route of administration utilized. See, e.g. Fingl et al., In: THE PHARMACOLOGICAL BASIS OF THERAPEUTICS, Ch. 1, p. 1, 1975. The exact formulation, route of administration, and dosage can be chosen by the individual physician in view of the patient's condition and the particular method in which the compound is used.

### VII. Examples

### Example 1

### Representative Synthesis of Compounds Described Herein

Peptides were synthesized on ABI 433A synthesizer with Fmoc chemistry. Preparative reverse-phase HPLC was performed on a Waters HPLC/MS system consisting of Waters 2525 Prep HPLC Pump, 2767 sample manager, 2487 dual absorbance detector and Micromass® ZQ™ mass spectrometer. The crude peptide was purified using Waters preparative HPLC/MS instrument with Kromasil C4 columns using a linear gradient (25-45%) of buffer B in buffer A over 30 min (buffer A=0.05% TFA in water; buffer B=0.05% TFA in ACN) and a flow rate of 20 mL/min. Analytical reverse-phase HPLC was performed on an Agilent 1100 system equipped with a 6120 quadrupole LC/MS.

Cmpd 14 (Cmpd 3-Lys(Cys(#)][#Mal-PEG40 KD-[des-Lys¹]-Cmpd 4).

Des-Lys¹-Cmpd 4 was prepared with regular Fmoc-amino acids on rink amide resin. Cmpd 3-Lys(Cys) amide was prepared with regular Fmoc-amino acids on rink amide resin except that the first residue from the C-terminal, Fmoc-Lys(Boc-Cys(trt))-OH, was purchased from EMD chemicals. Both peptides were purified and used in TFA salt form. The structure of Cmpd 3-Lys(Cys) amide is shown following:

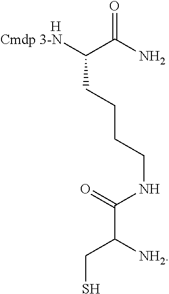

40K bifunctional PEG MA-400Ts was purchased from NOF corporation. The structure of MA-400Ts follows:

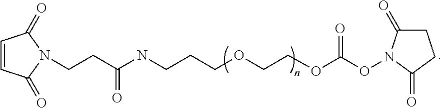

MA-400Ts (7.5 nmol) was dissolved in 10 mL anhydrous DMF. [Des-Lys¹]-Cmpd 4 (7.4 nmol) was added followed by 50 uL of diisopropylethylamine (DIEA). The reaction was left to sit overnight before tert-butyl methyl ether (TBDME) was added to precipitate the conjugate. The crude compound was washed twice with TBDME, dried, reconstituted in 15 ml of 20 mM pH 4.0 NaOAc buffer, and then purified by FPLC (Akta Explorer 100, GE) at pH 4.0 with a self-packed 15 mL Macrocap™ SP column (GE, gradient 0-30%-100%, 13CV each, 3 ml/min). The fall through fractions was recycled through a 5 ml HiTrap™ SP HP column (GE, gradient 0-20%-100%, 15 CV each, 2 ml/min). Fractions of the conjugate Des-Lys 1-Cmpd 4-PEG40K-Mal were pooled and used immediately for the next reaction except a small fraction of the pool was dialyzed against water and lyophilized to determine that the overall yield was 4.8 nmol (64.5% yield).

To the Des-Lys1-Cmpd 4-PEG40K-Mal (3.2 nmol) pool was added Cmpd 3-Lys(Cys) (3.2 nmol) and the mixture was left to sit overnight. The crude resultant was purified by FPLC at pH 4.0 with a self-packed 15 mL Macrocap™ SP column (from GE, gradient 0-100%, 30 CV, 3 ml/min). Fractions of the final product were pooled according to Maldi-TOF MS and SDS page Gel results, dialyzed and lyophilized to give 2.3 nmol (71.8% yield) of Cmpd 14.

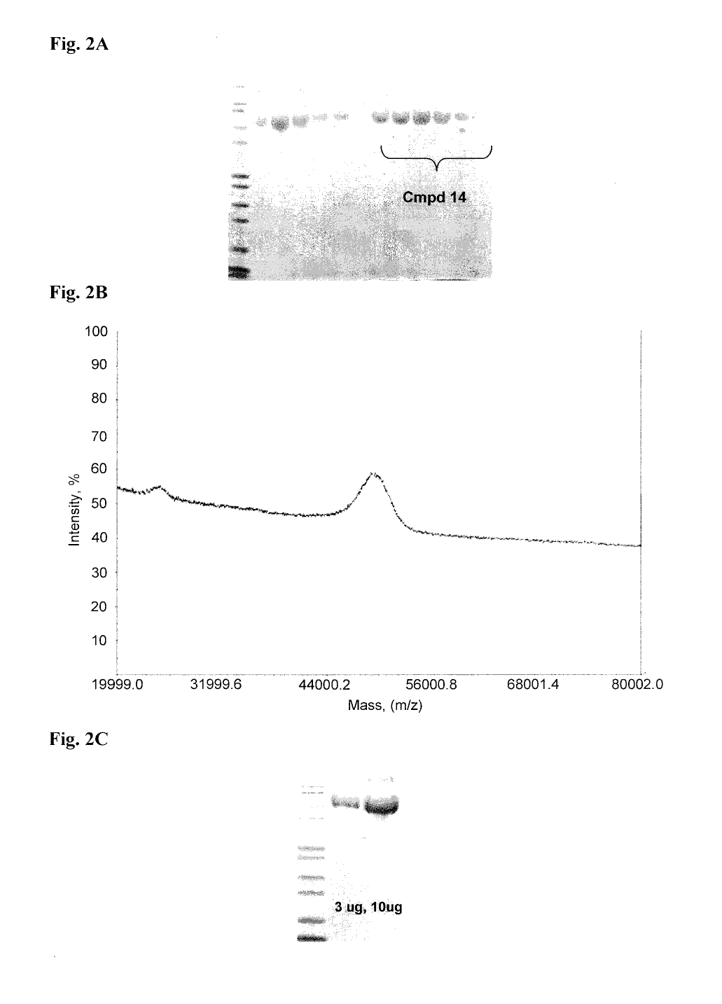

SDS polyacrylamide electrophoresis (SDS PAGE) employing NuPAGE® 4-12% Bis-Tris Gel was conducted on fractions from the FPLC purification of Cmpd 14 with 15 ml Macrocap™ SP column, the results of which are shown in FIG. 2A.

Mass spectrometry using the Maldi-TOF MS instrument was conducted on Cmpd 14, with results shown in FIG. 2B. Peaks are observed at 24929.73 and 49534.56 (m/z).

Another SDS PAGE electropherogram (NuPAGE® 4-12% Bis-Tris Gel) was conducted to demonstrate the effect on concentration of Cmpd 14, the results of which are shown in FIG. 2C.

### Example 2

### In Vitro Functional Assay: Cmpds 12-14

LDDHC compounds described herein, and appropriate control compounds, were investigated for activity in functional assays for GLP-1 and calcitonin as described herein. As shown in Table 3 following, LDDHCs (Cmpds 12-14) demonstrate activity in the nanomolar range in both GLP-1 and calcitonin functional assay. Furthermore and surprisingly, the size of the water-soluble polymeric spacer PS affects the in vitro activity, as evidence by a comparison of Cmpd 13 (10 kDa PEG spacer) with Cmpd 14 (40 kDa PEG spacer). GLP-1 cyclase assay data are normalized against control peptide: Exendin-4 EC₅₀=4 pM, GLP-1 EC₅₀=10 pM. Calcitonin cyclase assay data are normalized against control peptide: Davalintide EC₅₀=40 pM.

TABLE 3
In vitro GLP-1R and Calcitonin functional assay

| | GLP-1 function | Calcitonin function |
|---|---|---|
| Cmpd | (nmol) | (nmol) |
| 2 | 0.010 | |
| 3 | 0.011 | |
| 4 | | 0.040 |
| 5 | | 15.4 |
| 6 | | 1.23 |
| 7 | 0.073 | |
| 8 | 0.060 | 1.31 |
| 9 | 0.044 | 3.63 |
| 10 | 6.2 | 64.5 |
| 11 | 0.47 | 7.13 |
| 12 | 9.6 | 2.65 |
| 13 | 1.6 | 6.9 |
| 14 | 0.18 | 0.65 |

### Example 3

### In Vivo Investigations: Cmpd 14

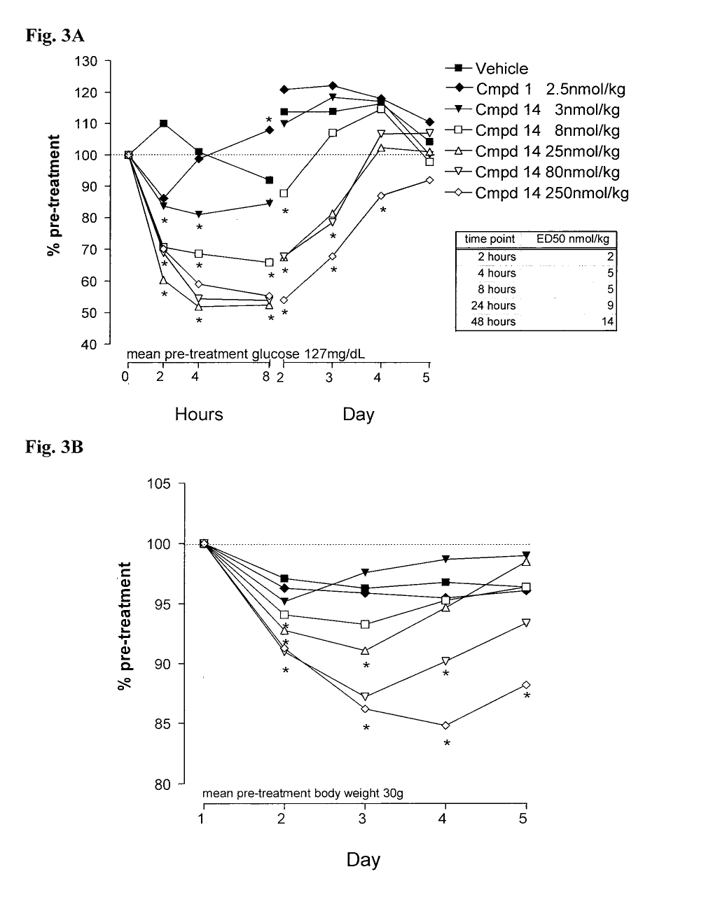

Dose dependent effects on the time course of blood glucose and body weight were measured for Cmpd 14 and control Cmpd 1 (Exendin-4). Assay conditions were are described herein. Test compound and vehicle were injected SC at zero time, immediately following baseline sampling. Subjects were NIH/Swiss female mice. Cmpd 14 was administered at 3, 8, 25, 80 and 250 nmol/kg. Samples were taken at t=2, 4 and 8 hrs during day 1, and then daily through day 5. Mean pre-treatment blood glucose was 127 mg/dL. As shown in FIGS. 3A-B, there is a dose dependent decrease in blood glucose and body weight for Cmpd 14 in these assays. “*” symbol indicates p<−0.05 vs. vehicle, using ANOVA with Dunnett's test.

### Example 4

### In Vivo Investigations: Cmpd 8 and Cmpd 9

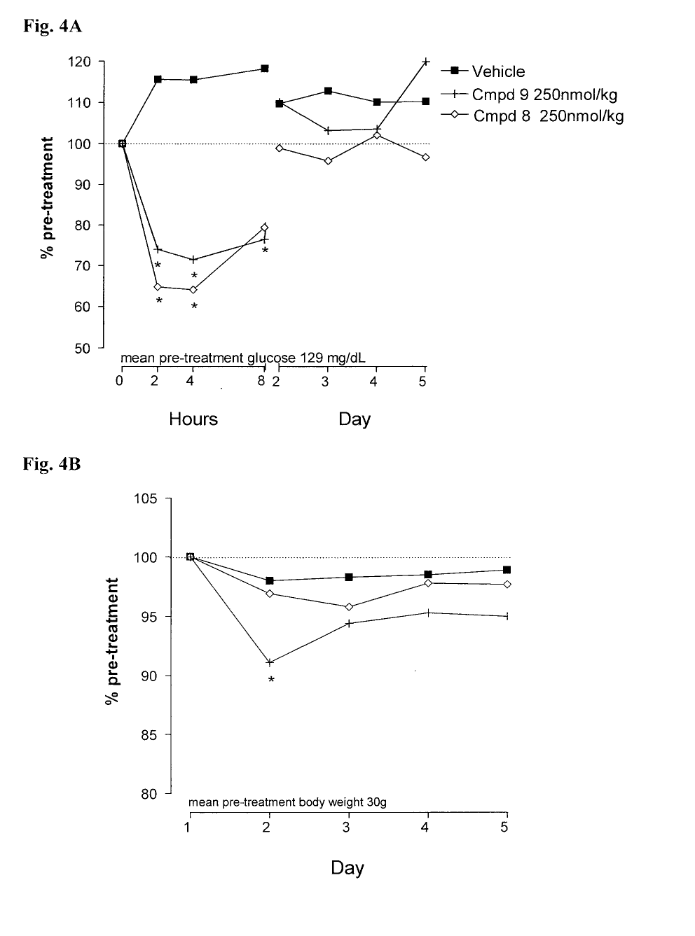

The linear hybrid compounds Cmpd 8 and Cmpd 9 were assayed for the time course of blood glucose and body weight. Assay conditions and experimental design were as described above. Test subjects were NSA female mice. The compounds were administered at 250 nmol/kg at zero time. The mean pre-treatment blood glucose was 129 mg/dL. As shown in FIGS. 4A-B, both Cmpd 8 and Cmpd 9 elicit an immediate decrease in blood glucose. As judged by FIGS. 4A-B, Cmpd 9 is more effective in decreasing body weight over 5 days compared to Cmpd 8.

### Example 5

### In Vivo Investigations: Cmpd 11 and Cmpd 14

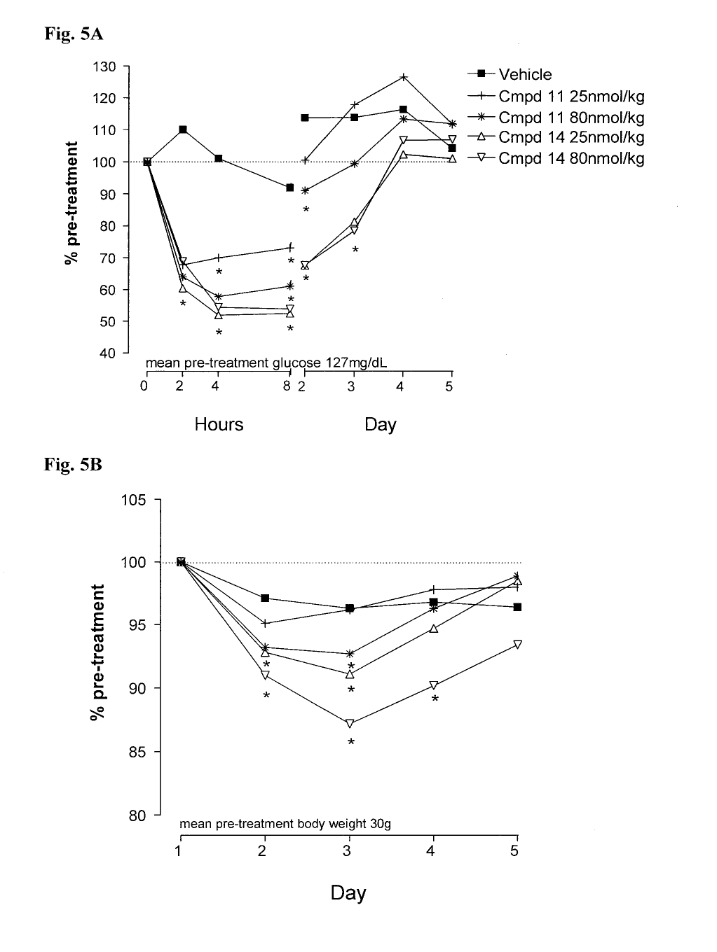

A comparison of the blood glucose and body weight changes with Cmpd 11 and Cmpd 14 at 25 and 80 nmol/kg dosing was conducted, using the experimental system described for Example 3. As shown in FIGS. 5A-B, Cmpd 14 is more effective than Cmpd 11 in reducing blood glucose and decreasing body weight through at least day 4.

### Example 6

### In Vivo Investigations: Cmpds 1, 5, 7, 14

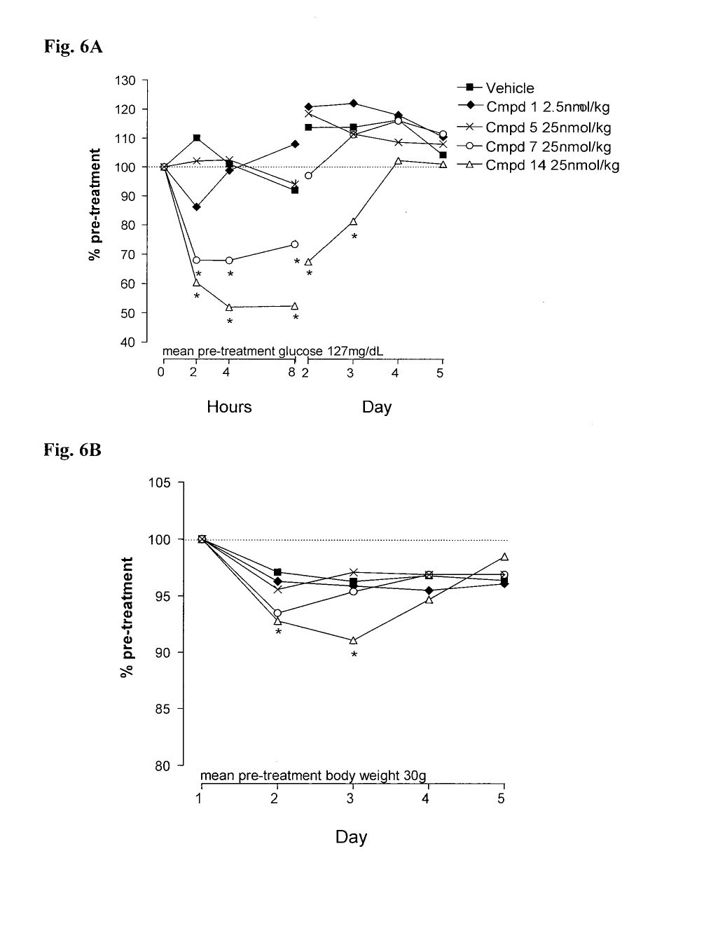

Studies were designed to investigate whether a synergistic or other non-additive effect can be observed with LDDHC compounds (e.g., Cmpd 14) relative to compounds having only a single biologically active peptide (Cmpd 5 and Cmpd 7), each in combination with a water-soluble polymeric spacer (PEG 40 kDa). Assay and experimental conditions for the blood glucose and body weight procedures were as described above. The test subjects were NIH/Swiss female mice. Compound dosing was at 25 nmol/kg, with the exception that control Cmpd I was administered at 2.5 nmol/kg. As shown in FIGS. 6A-B, only Cmpd 14 demonstrates a sustained effect in lowering blood glucose and body weight loss through days 2-3. The effect of Cmpd 5 and Cmpd 7 decreased by day 2.

### Example 7

### Duration of Food Intake and Body Weight Reduction: Cmpds 6, 7, 14

The effect of Cmpd 14 on body weight was examined in male outbred diet-induced obese Sprague Dawley rats. Percent vehicle-corrected change in body weight was measured relative to PEGylated parent peptides alone and in combination. Drug was administered subcutaneously in two separate single injections spaced one week apart. The treatment groups are listed in Table 4 following.

TABLE 4
Treatment paradigm for investigation of
Cmpd 14 on body weight in DIO rats.

| Group | Compound | Dose |
|---|---|---|
| 1 | Vehicle | 0 |
| 2 | Cmpd 7 | 32 nmol/kg (1.4 mg/kg) |
| 3 | Cmpd 6 | 32 nmol/kg (1.4 mg/kg) |
| 4 | Cmpd 7 + Cmpd 6 | Each at 32 nmol/kg |
| 5 | Cmpd 14 | 32 nmol/kg (1.5 mg/kg) |

Specifically, in this experiment the duration of action on food intake and change in body weight was compared for compounds having a single active peptide element (Cmpd 6 and Cmpd 7) relative to the combined dosing of these compounds, and further relative to LDDHC Cmpd 14, having the active peptide elements of Cmpds 6-7 separated by a 40 kDa PEG water-soluble polymeric spacer. All compounds were administered at 32 nmol/kg. In the combination dosing, each of Cmpd 6 and Cmpd 7 was administered at 32 nmol/kg. The test subjects were DIO rats (n=6/group, body weight baseline=478 g). Dosing occurred at days 0 and 7.

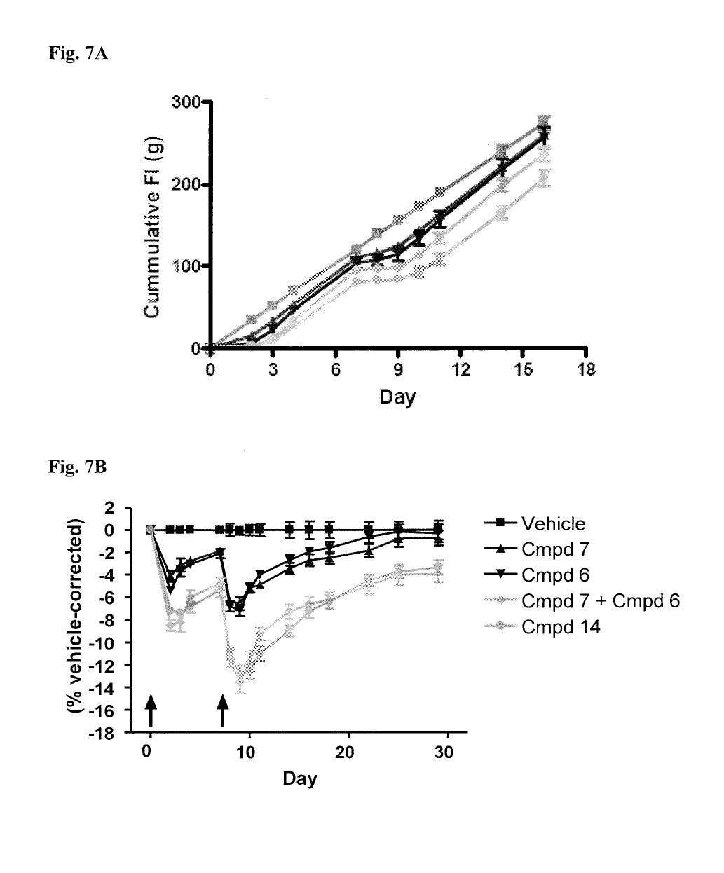

As shown in FIGS. 7A-B, a relatively small but statistically significant decrease in both food intake and body weight (% vehicle-corrected) is observed with Cmpd 14 relative to either Cmpd 6 or Cmpd 7, or to the combined administration thereof.

### Example 8

### Raw Body Weight Comparison: Cmpds 6, 7, 14

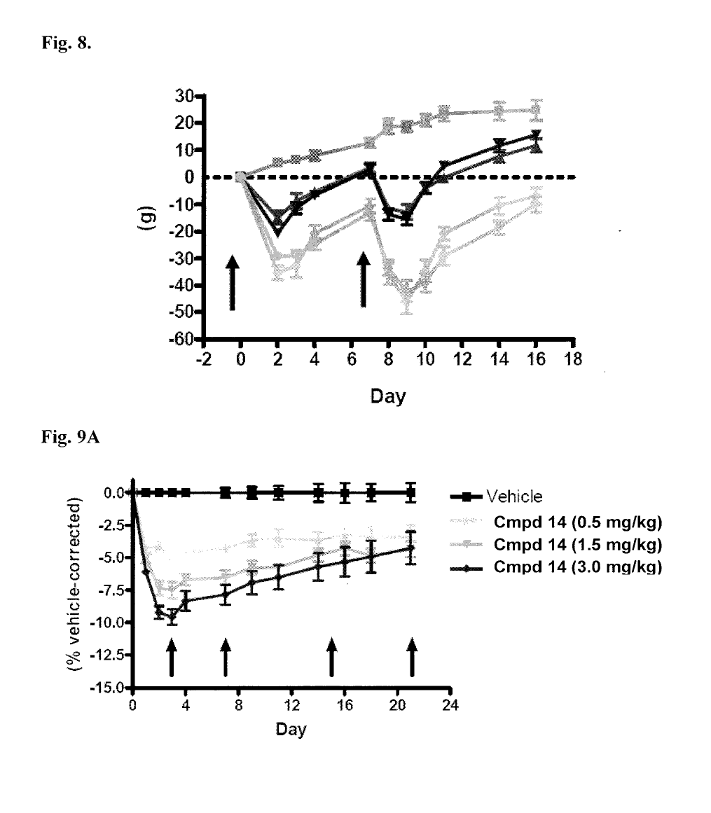

The experiment described in the previous Example was further analyzed for raw body weight. As shown in FIG. 8, raw body weight loss in DIO rats dosed with Cmpd 14 is additive of that induced by single element compound (Cmpd 6/cmpd 7) administration, being approximately equal to co-administration.

### Example 9

### Pharmacokinetic/Pharmacodynamic Results: Cmpd 14

Cmpd 14 PK ELISA for Rat Plasma.

An immunoassay was developed for the quantification of Cmpd 14 in plasma. This assay is a two-site “sandwich” immunoassay, which uses two monoclonal antibodies. Immulon™ 2HB microtiter plates were coated with an in-house mouse monoclonal antibody at 5 μg/mL in 0.2M carbonate buffer (100 μL/well) and incubated at 2-8° C. overnight. Plates were washed 3 times with PBS Tween to remove unbound antibody, and wells were blocked with 3% BSA for 1-2 hours. A frozen standard prepared at 102,400 pg/mL in rat plasma was serially diluted 1:2 in rat plasma on the day of the assay to generate the calibration curve. Excess 3% BSA block was washed off, and samples were added in duplicate to the wells with a minimum required dilution of 1:3 (total volume of 150 μL/well). Samples expected to be higher than upper limit of quantification (ULOQ) were diluted in Immunochemistry Technologies SDI buffer into the assay range. Low, Mid, High and dilutional controls were run on each plate as assay acceptance controls. After a 1-2 hour incubation, the plates are washed and a horse-radish peroxidase (HRP) conjugated in-house monoclonal antibody is added for another 1-2 hours. Excess HRP is washed off and colorimetric substrate, TMB, is added. The reaction is stopped with 0.3M H₂SO₄ when the high standard reaches 0.8-1.0 OD₆₅₀. The data is analyzed with Softmax® Pro with a 5-parameter curve fit.

The pharmacokinetics and pharmacodynamics of Cmpd 14 upon a single dose was investigated over a 21-day period. Dosing was at 0.5, 1.5 and 3.0 mg/kg. As shown in FIG. 9A, there is a dose dependent decrease in body weight (% vehicle corrected) with increasing dosage. Plasma drug levels were determined by ELISA assay. As shown in FIG. 9B, there is an approximately first-order clearance of Cmpd 14 under these experimental conditions.

The data depicted in FIG. 9B are tabulated in Table 5 following. In Table 5, change in body weight and corresponding plasma levels of Cmpd 14 in dose-response at days 3, 7, 14 and 21 post injection to DIO Sprague Dawley rats, n=6/group (except where indicated), are provided. For plasma drug levels on day 21 only a single animal in the high-dose group demonstrated a measurable levels of Cmpd 14 and the data is not shown. Maximal body weight loss was observed at the day 3 time point. BW, body weight.

TABLE 5

| | Day 3 Δ | Plasma | Day 7 Δ | Plasma | Day 14 Δ | Plasma | Day 21 Δ |
|---|---|---|---|---|---|---|---|
| | BW (%, | drug | BW (%, | drug | BW (%, | drug | BW (%, |
| | vehicle- | levels | vehicle- | levels | vehicle- | levels | vehicle- |
| Dose | corrected) | (ng/mL) | corrected) | (ng/mL) | corrected) | (ng/mL) | corrected) |
| Vehicle | 0.0 ± 0.2 | ND | 0.0 ± 0.4 | ND | 0.0 ± 0.7 | ND | 0.0 ± 0.7 |
| 0.5 mg/kg | −4.9 ± 0.3 | 233 ± 24 | −4.2 ± 0.3 | 10 ± 2 | −3.7 ± 0.7 | ND | −3.4 ± 0.9 |
| 1.5 mg/kg | −7.5 ± 0.7 | 730 ± 72 | −6.5 ± 0.6 | 37 ± 6 | −4.8 ± 0.5 | 0.5 ± 0.1 | −4.3 ± 0.6 |
| | | | | | | (n = 2) | |
| 3.0 mg/kg | −9.6 ± 0.6 | 1377 ± 166 | −7.9 ± 0.8 | 80 ± 19 | −5.7 ± 1.1 | 11.3 ± 0.2 | −4.2 ± 1.3 |
| | | | | | | (n = 2) | |

### Example 10

### Pharmacodynamic and Vacuolation Assay: Cmpds 6, 7, 14

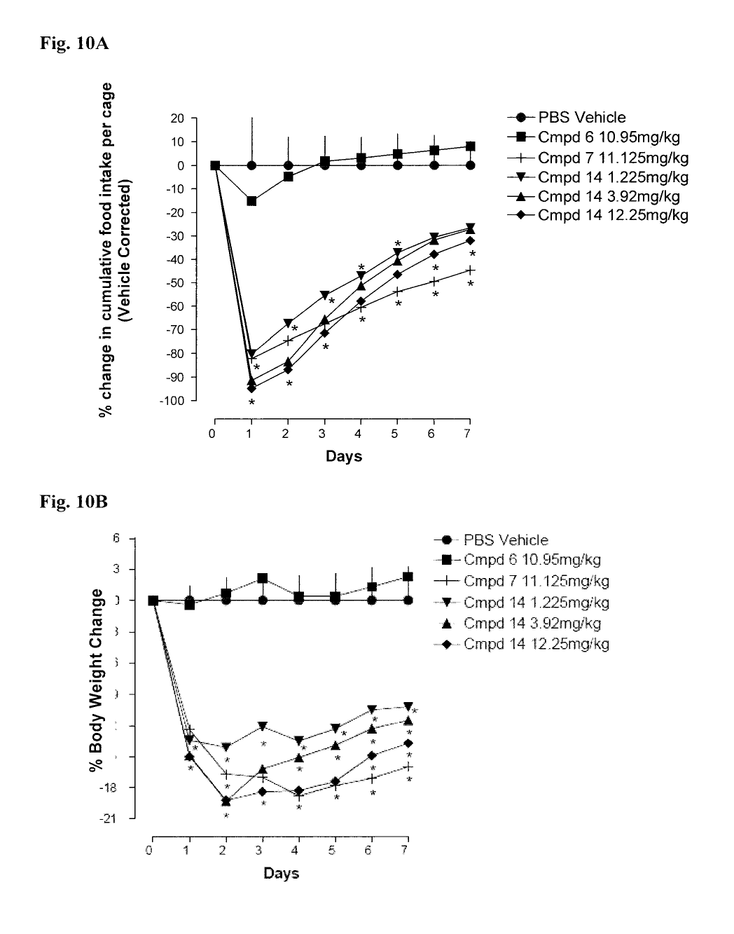

In an experiment parallel to the previous experiment, the pharmacodynamic and kidney histology (vacuolation) results of administration of pegylated compounds described herein was conducted. The experiment followed the procedures described in the previous Example. Cumulative food intake and body weight changes were determined in the test cohort. See FIGS. 10A-10B, with dosing and duration as indicated. Cmpd 6 demonstrated negligible reduction in cumulative food intake and body weight change. Administration of Cmpd 7 or Cmpd 14 resulted in both decreased cumulative food intake and body weight.

Test subjects were sacrificed on day 7, and the kidneys were excised and submitted for histopathological analysis, with results shown in Table 6 following. Vacuolation analyses were conducted as described above. Administration of Cmpds 6, 7 or 14 resulted in no kidney vacuolation as judged by a histological examination.

TABLE 6
Kidney vacuolation assay results

| Cmpd | Individual Vacuole Score | Average Vacuole Score |
|---|---|---|
| 6 | 0, 0, 0, 0, 0, 0 | 0 |
| 7 | 0, 0, 0, 0, 0, 0 | 0 |
| 14 | 0, 0, 0, 0, 0, 0 (all doses) | 0 (all doses) |

### Example 11

### In Vitro Functional Assay

The effect of the size of the water soluble (PEG) moiety on LDDHC analogs of Cmpd 14 was investigated. The in vitro function in the GLP-1 and calcitonin assay of Cmpds 45, 46 and 47, which differ from Cmpd 14 in having PEG moieties of 30K, 60K and 80K, respectively, were determined by methods described herein. In these studies, the GLP-1 cyclase assay data were normalized against control peptide: Exendin-4 (EC₅₀=4 pM), GLP-1 (EC₅₀=10 pM). Calcitonin cyclase assay data were normalized against control peptide: Davalintide (EC₅₀=40 pM). Results of these assay are tabulated in Table 7 following.

TABLE 7
In vitro functional assay of compounds related to Cmpd 14.

| | GLP-1 function | Calcitonin function |
|---|---|---|
| Cmpd | (nmol) | (nmol) |
| 45 | 0.17 | 3.58 |
| 46 | 0.17 | 3.11 |
| 47 | 0.43 | 4.42 |

### Example 12

### In Vitro Binding and Functional Assays

Binding and functional assays for Cmpds 4, 9, 48 and 14 and were conducted to determine CGRP binding, adrenomedullin (AM) receptor binding, AM cyclase EC₅₀, and AM cyclase IC₅₀ for these compounds. Methods were as described herein or known in the art.

TABLE 8

| | CGRP binding | AM binding | AM cyclase | AM cyclase |
|---|---|---|---|---|
| Cmpd | IC₅₀ (nM) | IC₅₀ (nM) | EC₅₀ (nM) | IC₅₀ (nM) |
| 4 | 4.3 ± 2.3 (24) | 1.2 ± 0.5 (3) | >10000 (5) | 18 ± 7 (4) |
| 9 | 1166 ± 368 (2) | 543 (1) | >10000 (3) | >10000 (2) |
| 48 | 760 (1) | >10000 (1) | >10000 (2) | >10000 (1) |
| 14 | >1000 (1) | >10000 (1) | >10000 (2) | >10000 (1) |

As shown in Table 8, davalintide (Cmpd 4) displays a high potency for both the CGRP receptor and the adrenomedullin receptor. In contrast, Cmpds 9, 48 and 14, having a davalintide component, are nonetheless significantly less potent at both receptors. Indeed, Cmpd 4 is an antagonist in the adrenomedullin cyclase assay, whereas Cmpds 9, 48 and 14 are not. Despite davalintide being a potent adrenomedullin receptor antagonist (IC₅₀=18 nM), Cmpds 48 and 14 did not display functional activation or antagonism of the adrenomedullin receptor at concentrations up to 10 uM. Accordingly, Cmpds 9, 48 and 14 presents a surprisingly different pharmacological profile compared to davalintide with respect to cellular receptors that recognize amylin and amylinomimetics. Thus, Cmpds 9, 48 and 14 will have fewer off-target activities than the parent peptide. The improved pharmacological profiles for Cmpds 9, 48 and 14 are expected to result in decreased side-effects, such as reduced severe flushing, nausea and/or vomiting, particularly with human subjects, as compared to the parent peptide davalintide. For example, CGRP and CGRP agonists have been reported to induce severe flushing and even nausea and vomiting in human subjects, which is believed in part due to activation of CGRP receptors and which is relieved by CGRP antagonists. It is expected that Cmpds 9, 48 and 14 will have increased patient compliance and/or allow increased dosing as needed compared to previous compounds, for example compared to davalintide, resulting in improved commercial success.

### Example 13

### Effect of PEG Size on Duration of Action

In order to assess the impact of the size of the PEG included within the compounds described herein, a comparison of the effects of a single dose of compound over time on body weight was conducted using Cmpds 45, 46, 47, 14a and 14. See Table 9 following. Cmpd 14a has the formal structure of Cmpd 14 and differs from Cmpd 14 in having a different 40 kDa PEG reagent used in for synthesis. Synthesis of Cmpds 45, 46, 47, and 14 used the 40 kDa PEG reagent from JenKem, whereas synthesis of Cmpd 14a employed the 40 kDa PEG reagent from NOF.

TABLE 9

| Group | Cmpd | Size of PEG | Dose |
|---|---|---|---|
| 1 | Vehicle | NA | PBS |
| 2 | 45 | 30 kDa | 1.22 mg/kg |
| 3 | 46 | 60 kDa | 2.07 mg/kg |
| 4 | 47 | 80 kDa | 2.84 mg/kg |
| 5 | 14a | 40 kDa | 1.57 mg/kg |
| 6 | 14 | 40 kDa | 1.5 mg/kg |

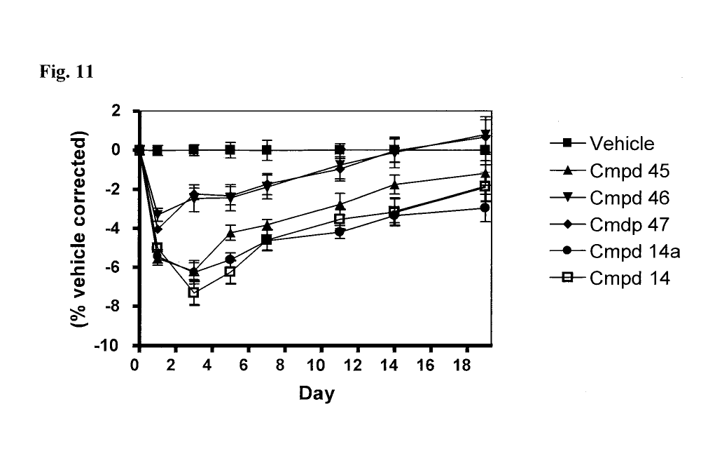

Male Sprague Dawley DIO rats (n=6) were administered a single dose of a compound listed in Table 9, and body weight was monitored over 19-days. The baseline body weight was 505 gm. As shown in FIG. 11, Cmpds 14 and 14a, having 40 kDa PEG moieties, were approximately equally efficacious in reducing body weight under these conditions. Significantly less efficacious were Cmpd 45 (30 kDa PEG), Cmpd 46 (60 kDa PEG), and Cmpd 47 (80 kDa PEG).

### Example 14

### Change in Body Weight—Cmpds 48 and 14

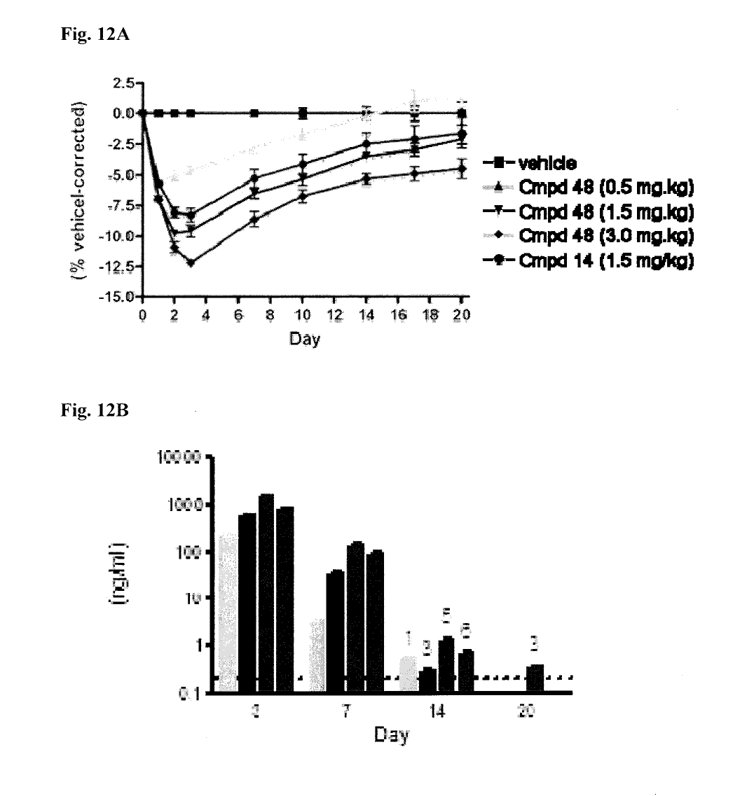

The effects of dose on weight loss were determined for Cmpd 48 under the conditions described in Example 13. As shown in FIG. 12A, there is a dose response for weight loss as the dose of Cmpd 48 increases. FIG. 12B depicts a histogram of plasma drug levels at 3, 7, 14 and 20 days.

### Example 15

### Preparation of Compounds

Compounds.

Compounds described herein were synthesized by several methods, including the following exemplary methods.

a). For example, Cmpd 67 was prepared by treating mPEG40K-aldehyde with the N-terminal of Cmpd 49 in a reductive alkylation reaction to generate specifically N-terminal pegylated Cmpd 49.

b). In another example, Cmpd 68 was prepared by reacting the N-terminal amino group of Cmpd 50 with an mPEG40K-NHS (n-hydroxysuccinimide ester).

c). In another example, Cmpds 69, 71, 73, and 75 were prepared as shown in Scheme 15 following.

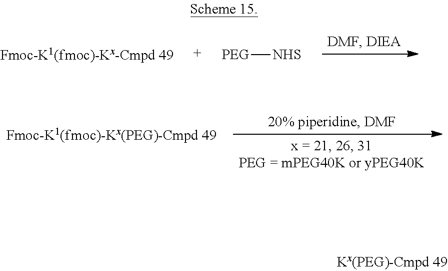

Analogs of Cmpd 49 with Fmoc protected Lys¹ and a mutated lysine at the 21, 26 and 31 positions were treated with mPEG40K-NHS in DMF with DIEA. The resulting pegylated peptide was deprotected by piperidine to give the pegylated free peptide.

d) In another example, Cmpd 74, 70, and 72 were prepared by selective pegylation on a lysine side-chain. Analogs of Cmpd 50 with a mutated lysine at positions 21, 24-29 and 31 were treated with mPEG40K-NHS in DMF with DIEA. The crude product was purified and analyzed for regio-specificity.

### Example 16

### Receptor Binding Activity

Method.

A receptor binding activity assay was conducted using the compounds described herein. Receptor binding activity can be expressed, for example in Table 10, as an IC₅₀ value, calculated from the raw data using an iterative curve-fitting program using a 4-parameter logistic equation (PRISM®, Graph PAD Software, La Jolla, Calif.), as known in the art.

For the amylin receptor binding assay, RNA membranes were incubated with approximately 20 pM (final concentration) of ¹²⁵I-rat amylin (Bolton-Hunter labeled, PerkinElmer, Waltham, Mass.) and increasing concentrations of test compound for 1 hour at ambient temperature in 96-well polystyrene plates. Bound fractions of well contents were collected onto a 96 well glass fiber plate (pre-blocked for at least 30 minutes in 0.5% PEI (polyethyleneimine)) and washed with 1×PBS using a Perkin Elmer plate harvester. Dried glass fiber plates were combined with scintillant and counted on a multi-well Perkin Elmer scintillation counter, as well known in the art.

Results.

As shown in Table 10 following, the pegylated compound can be generally less potent than the corresponding non-pegylated polypeptide component (Cmpd 49) in an amylin binding assay. Removal of the N-terminal lysine of parent Cmpd 49 to provide Cmpd 50 and pegylation of the resulting compound appears to reduce all binding activity. It further appears that derivatization at any of positions of 21, 24-29 and 31 is detrimental to receptor binding.

TABLE 10
Receptor Binding Assay

| | | | Binding | | |
|---|---|---|---|---|---|
| | | | IC₅₀ (nM) | | Functional |
| | | | Rat | | EC₅₀ (nM) |
| | SEQ ID | PEG | Nucleus | Human | Human |
| Cmpd | NO. | site/type | Accumbens | AMY3 | AMY3 |
| 49 | 74 | | 0.10 | 0.247 | 0.003 |
| 50 | 75 | | 0.42 | | |
| 67 | 92 | N-term | 40 | | |
| 68 | 93 | N-term | 78 | | |
| 69 | 94 | 21 | 104 | | |
| 71 | 96 | 26 | 66 | | |
| 73 | 98 | 31 | 86 | | |
| 75 | 100 | 26 | 470 | | |
| 74 | 99 | 31 | 75 | | |
| 70 | 95 | 21 | 112 | | |
| 72 | 97 | 26 | 54 | | |
| 76 | 101 | 21 | 131 | 388 | 1.75 |
| 77 | 102 | 26 | 39 | 60 | 0.078 |
| 78 | 103 | 31 | 61 | 80 | 0.10 |
| 79 | 104 | 26/yPEG | 132 | 1132 | 1.74 |
| 80 | 105 | 24 | 79 | 111 | 0.41 |
| 81 | 106 | 25 | 31 | 48 | 0.086 |
| 82 | 107 | 27 | 51 | 87 | 0.20 |
| 83 | 108 | 28 | 53 | 46 | 0.20 |
| 84 | 109 | 29 | 27 | 47 | 0.11 |
| 85 | 110 | 22 | 148 | | 1.66 |
| 86 | 111 | 23 | 611 | 240 | 2.82 |
| 87 | 112 | 30 | 599 | 685 | 4.25 |
| 88 | 113 | 26 | | 74 | 0.082 |
| 89 | 114 | 17 | | 334 | 2.23 |
| 90 | 115 | 18 | | 238 | 0.85 |
| 91 | 116 | 20 | | 918 | 5.51 |
| 92 | 117 | 32 | | 1000 | 24.69 |
| 93 | 118 | 33 | | 860 | 1.69 |
| 94 | 119 | 34 | | 77 | 0.034 |
| 95 | 120 | 35 | | 143 | 0.122 |
| 96 | 121 | 36 | | 984 | 3.074 |

### Example 17

### Effect of Pegylation on Food Intake: Cmpds 69, 73, 72, 70, 74

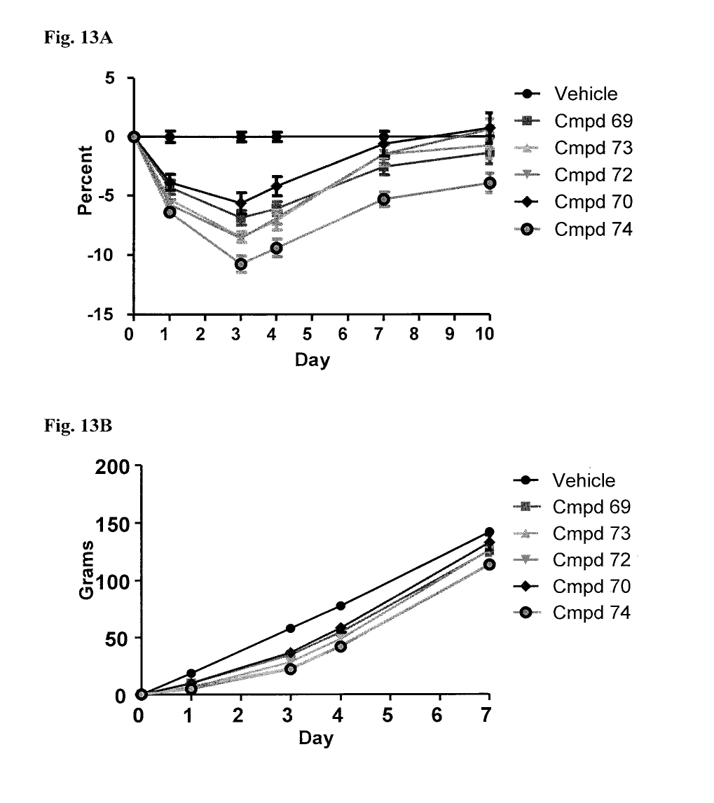

Lean rats were administered a subcutaneous (SC) once weekly injection of test compound (125 nmol/kg) or vehicle. FIGS. 13A-13B provide the result of a multi-day food intake assays. The effect on 24-hour food intake was investigated for Cmpds 69, 73, 72, 70, and 74, using vehicle as control. The results of FIGS. 13A-B demonstrate that each of the tested compounds was efficacious in reducing body weight and food intake for three days. In the case of some of the compounds, weight loss was still evident even after one week.

### Example 18

### Effect of Pegylation on Food Intake: Cmpds 74, 71

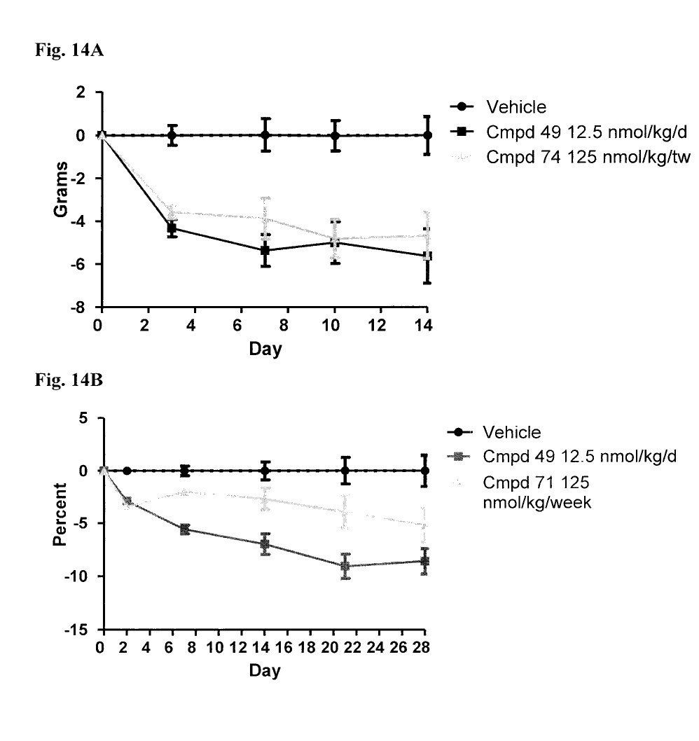

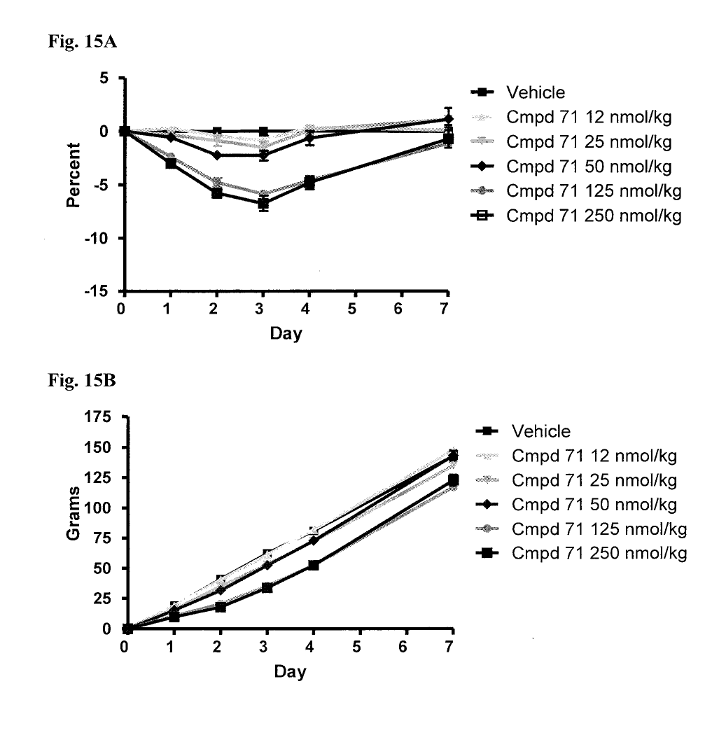

The effect on weight loss, as judged with SC injection, of a twice a week dose or once a week dose of Cmpd 74, was investigated. When dosed twice a week at 125 nmol/kg in a DIO (“diet-induced obese”) rat, Cmpd 74 has similar efficacy as a continuous infusion of 12.5 nmol/kg/d Cmpd 49 (FIG. 14A). Cmpd 71 dosed once a week at 125 nmol/kg was not as efficacious as infused Cmpd 49 when given to DIO rats for four weeks, but did show consistent lowering of body weight (FIG. 14B). Cmpd 71 also reduced body weight and food intake in a dose dependent fashion in lean rats, as shown in FIGS. 15A-15B.

### Example 19

### Effect of Pegylation on Food Intake: Cmpds 67, 71, 75

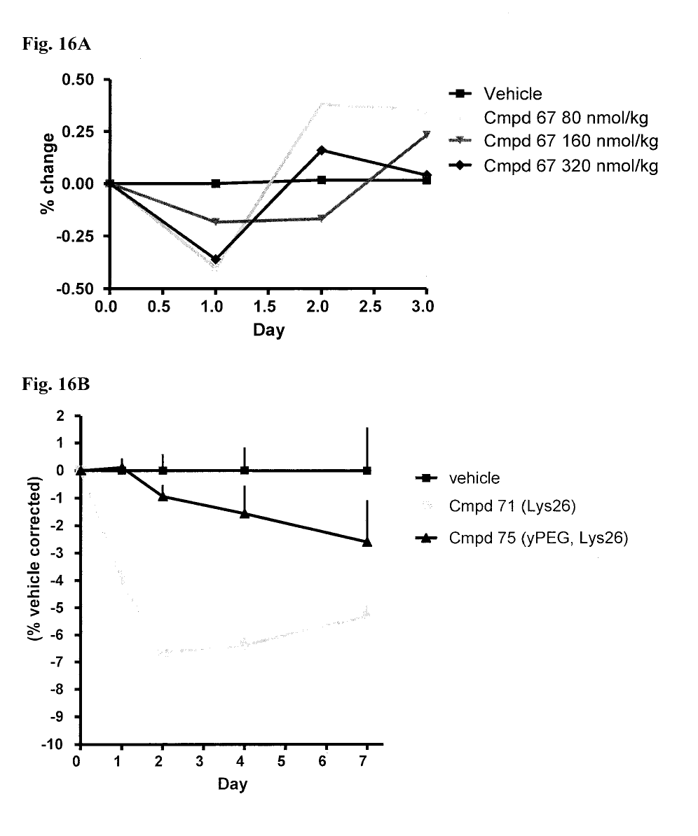

The effect on 24-hour food intake, as judged with SC injection, of a single dose of a compound having either a y-branched PEG (Cmpd 75) or an N-terminal PEG (Cmpd 67), was investigated. As shown in FIG. 16A, three doses of the N-terminal pegylated compound, Cmpd 67, were not as efficacious as the vehicle in reducing body weight in DIO rats. The y-branched pegylated compound, Cmpd 75, was not as efficacious as the linear pegylated version, Cmpd 71, in reducing body weight in lean rats, as shown in FIG. 16B.

### Example 20

### Effect of Pegylation on Food Intake: Cmpds 74, 76, 77, 78, 79

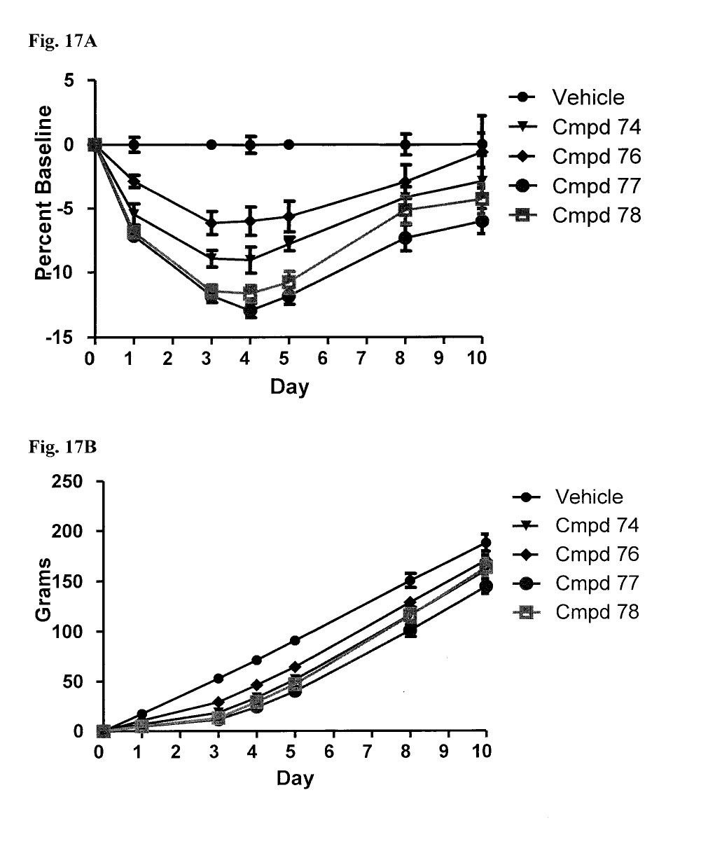

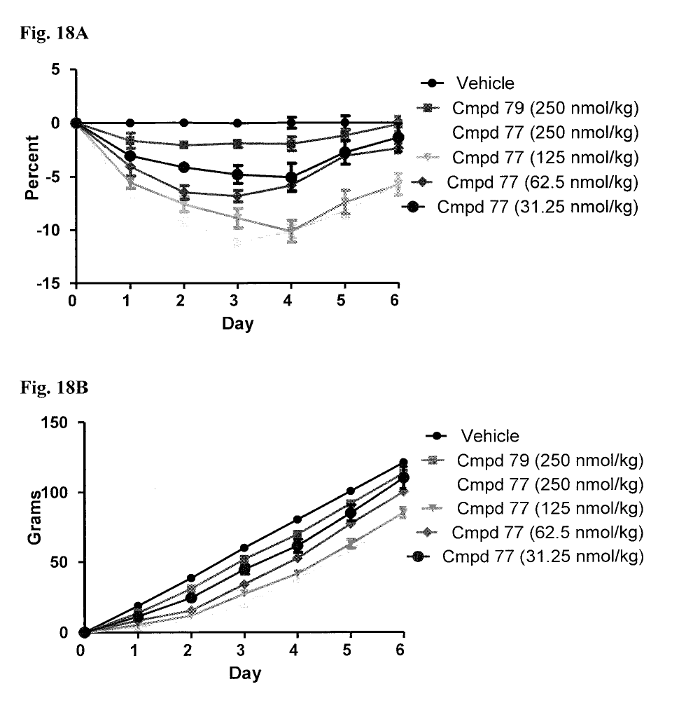

The effect on 24-hour food intake, as judged with SC injection (125 nmol/kg), was investigated for Cmpds 74, 76, 77 and 78. As shown in FIG. 17A-17B, each of the tested pegylated Cmpds 76, 77, and 78 were at least as efficacious as Cmpd 74 in body weight and food intake reduction in lean rats. The y-branched pegylated compound, Cmpd 79, was not as efficacious as the linear pegylated version, Cmpd 77, in body weight and food intake reduction in lean rats, as shown in FIG. 18A-18B. Cmpd 77 also showed dose dependent efficacy, as demonstrated in FIG. 18A-18B.

### Example 21

### Effect of Pegylation Position on Food Intake: Cmpds 77, 80-84

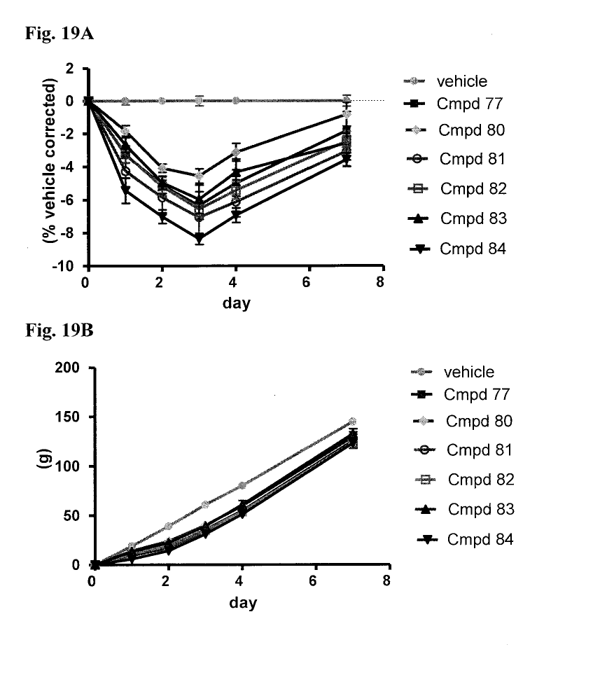

The effect on cumulative food intake and body weight reduction was investigated for Cmpds 77 and 80-84 (125 nmol/kg), having 40 kDa PEG at positions 26, 24, 25, 27, 28 and 29, respectively. Test animals (lean rats) were injected SC at 125 nmol/kg, and body weight corrected for vehicle (FIG. 19A) and cumulative food intake (FIG. 19B) were determined for 7-days. As shown in FIGS. 19A-19B, most compounds in the tested series provided weight loss which was still evident after 7-days.

In summary, the food intake data set forth in Examples 16-21 provides valuable observations regarding the efficacy and effect on duration of action of pegylation of the polypeptide element of the tested compounds. Specifically, 40 KD PEG derivatives of polypeptide components exhibit an extended time course of action compared to the non-pegylated peptide. The attachment of the PEG at positions 21, 26, or 31 increased both duration of action and the magnitude of the food intake response. Moreover, linear PEG compounds demonstrate greater efficacy in the food intake assay compared to the branched PEG compounds.

### VIII. Embodiments

### Embodiment 1

A long-duration dual hormone conjugate (LDDHC) compound having the structure of Formula I

P₁-L₁-PS-L₂-P₂ (I)

wherein P₁ is a peptide hormone having a first biological activity; P₂ is a peptide hormone having a second biological activity; L₁ and L₂ are independently a bond or a linker; and PS is a water-soluble polymeric spacer having a molecular weight in the range 30-80 kDa; wherein the compound exhibits the first biological activity in a biological assay, and the compound exhibits the second biological activity in a biological assay.

### Embodiment 2

The compound according to embodiment 1, wherein P₁ is an exendin, an exendin analog or a derivative thereof.

### Embodiment 3

The compound according to either of embodiments 1 to 2, wherein P₂ is an amylin, pramlintide, davalintide, or an analog or a derivative thereof.

### Embodiment 4

The compound according to any one of embodiments 1 to 3, wherein the exendin, exendin analog or derivative thereof is exendin-4, exendin analog-4 or derivative thereof.

### Embodiment 5

The compound according to any one of embodiments 1 to 4, wherein P₁ includes 1 to 39 residues.

### Embodiment 6

The compound according to embodiment 5, wherein P₁ includes 1 to 28 residues.

### Embodiment 7

The compound according to any one of embodiments 1 to 6, wherein P₂ comprises an amino acid sequence of residues 1-37 (SEQ ID NO:31) of Formula (II):

(II)
X′-Xaa¹-Cys²-Asn³-Thr⁴-Ala⁵-Thr⁶-Cys⁷-Ala⁸-Thr⁹-
Gln¹⁰-Arg¹¹-Leu¹²-Ala¹³-Asn¹⁴-Phe¹⁵-Leu¹⁶-Val¹⁷-
His¹⁸-Ser¹⁹-Ser²⁰-Xaa²¹-Asn²²-Phe²³-Xaa²⁴-Xaa²⁵-
Xaa²⁶-Xaa²⁷-Xaa²⁸-Xaa²⁹-Thr³⁰-Xaa³¹-Val³²-Gly³³-
Ser³⁴-Asn³⁵-Thr³⁶-Tyr³⁷-X

wherein up to 55% of the amino acids set forth in Formula (II) may be deleted or substituted with a different amino acid; wherein X′ is hydrogen, an N-terminal capping group, a bond to PS, or a linker to PS; Xaa¹ is Lys or a bond; Xaa²¹ is Lys, Cys, or Asn; Xaa²⁴ is Lys, Cys, or Gly; Xaa²⁵ is Lys, Cys, or Pro; Xaa²⁶ is Lys, Cys, or Ile; Xaa²⁷ is Lys, Cys, or Leu; Xaa²⁸ is Lys, Cys, or Pro; Xaa²⁹ is Lys, Cys, or Pro; Xaa³¹ is Lys, Cys, or Asn; and X is optional, and if present is substituted or unsubstituted amino, substituted or unsubstituted alkylamino, substituted or unsubstituted dialkylamino, substituted or unsubstituted cycloalkylamino, substituted or unsubstituted arylamino, substituted or unsubstituted aralkylamino, substituted or unsubstituted alkyloxy, substituted or unsubstituted aryloxy, substituted or unsubstituted aralkyloxy, hydroxyl, a bond to PS, or a linker to PS; wherein PS is covalently linked, optionally through a linker, to a side chain of a linking amino acid residue, X′ or X.

### Embodiment 8

The compound according to embodiment 7, wherein the linking amino acid residue is cysteine or lysine.

### Embodiment 9

The compound according to any one of embodiments 7 to 8, wherein PS is attached to the side chain of the amino acid at position 11, 24-29, or 31 of the compound comprising the structure of Formula (II).

### Embodiment 10

The compound according to any one of embodiments 1 to 9, wherein PS has a mass of 30-80 kDa.

### Embodiment 11

The compound according to embodiment 10, wherein PS has a mass of 35-60 kDa.

### Embodiment 12

The compound according to embodiment 10, wherein PS has a mass of about 40 kDa.

### Embodiment 13

The compound according to any one of embodiments 1o 12, wherein PS is a polyethylene glycol or a derivative thereof.

### Embodiment 14

The compound according to embodiment 13, wherein the polyethylene glycol is linear, branched or comb type.

### Embodiment 15

The compound according to embodiment 14, wherein the polyethylene glycol has a mass of 30-80 kDa.

### Embodiment 16

The compound according to embodiment 15, wherein the polyethylene glycol has a mass of 35-60 kDa.

### Embodiment 17

The compound according to embodiment 16, wherein the polyethylene glycol has a mass of about 40 kDa.

### Embodiment 18

A pharmaceutical composition comprising a compound according to any one of embodiments 1 to 17 in combination with a pharmaceutically acceptable excipient.

### Embodiment 19

A method for treating a disease or disorder in a subject, comprising administering a polypeptide conjugate according to any one of embodimentsl to 18 to a subject in need thereof in an amount effective to treat the disease or disorder.

### Embodiment 20

The method according to embodiment 19, wherein the disease or disorder is diabetes, type 1 diabetes, type 2 diabetes, obesity, hypertension, atherosclerosis, dyslipidemia, congestive heart failure, stroke, hypercholesterolemia, cardiovascular disease, myocardial ischemia, myocardial reperfusion, an eating disorder, gestational diabetes, diabetic neuropathy, pulmonary hypertension or insufficient pancreatic beta cell mass.

### Embodiment 21

The method according to embodiment 20, wherein the disease or disorder is diabetes, type 1 diabetes, type 2 diabetes or gestational diabetes.

### Embodiment 22

The method according to embodiment 20 wherein the disease or disorder is obesity.

### Embodiment 23

The method according to embodiment 20, wherein the disease or disorder is hypertension, atherosclerosis, congestive heart failure, stroke, cardiovascular disease, myocardial ischemia, myocardial reperfusion or pulmonary hypertension.

### Embodiment 24

The method according to embodiment 20, wherein the disease or disorder is dyslipidemia or hypercholesterolemia.

### Embodiment 25

A long-duration dual hormone conjugate (LDDHC) compound having the structure of Formula I

P₁-L₁-PS-L₂-P₂ (I)

wherein P₁ has a first biological activity and is an exendin, exendin analog or derivative thereof; P₂ has a second biological activity and is an amylin, amylin analog or derivative thereof; L₁ and L₂ are independently a bond or a linker; and PS is a water-soluble polymeric spacer having a molecular weight in the range 30-80 kDa; wherein the compound exhibits the first biological activity in a biological assay, and the compound exhibits the second biological activity in a biological assay.

### Embodiment 26

The compound of embodiment 25, wherein the exendin, exendin analog or derivative thereof is exendin-4, exendin-4 analog or derivative thereof.

### Embodiment 27

The compound of any one of embodiments 25 to 26, wherein P₂ comprises an amino acid sequence of residues 1-37 (SEQ ID NO:31) of Formula (II):

(II)
X′-Xaa¹-Cys²-Asn³-Thr⁴-Ala⁵-Thr⁶-Cys⁷-Ala⁸-Thr⁹-
Gln¹⁰-Arg¹¹-Leu¹²-Ala¹³-Asn¹⁴-Phe¹⁵-Leu¹⁶-Val¹⁷-
His¹⁸-Ser¹⁹-Ser²⁰-Xaa²¹-Asn²²-Phe²³-Xaa²⁴-Xaa²⁵-
Xaa²⁶-Xaa²⁷-Xaa²⁸-Xaa²⁹-Thr³⁰-Xaa³¹-Val³²-G1y³³-
Ser³⁴-Asn³⁵-Thr³⁶-Tyr³⁷-X,

wherein up to 55% of the amino acids set forth in Formula (II) may be deleted or substituted with a different amino acid; wherein X′ is hydrogen, an N-terminal capping group, a bond to PS, or a linker to PS; Xaa¹¹ is Lys or a bond; Xaa²¹ is Lys, Cys, or Asn; Xaa²⁴ is Lys, Cys, or Gly; Xaa²⁵ is Lys, Cys, or Pro; Xaa²⁶ is Lys, Cys, or Ile; Xaa²⁷ is Lys, Cys, or Leu; Xaa²⁸ is Lys, Cys, or Pro; Xaa²⁹ is Lys, Cys, or Pro; Xaa³¹ is Lys, Cys, or Asn.; and X is optional, and if present is substituted or unsubstituted amino, substituted or unsubstituted alkylamino, substituted or unsubstituted dialkylamino, substituted or unsubstituted cycloalkylamino, substituted or unsubstituted arylamino, substituted or unsubstituted aralkylamino, substituted or unsubstituted alkyloxy, substituted or unsubstituted aryloxy, substituted or unsubstituted aralkyloxy, hydroxyl, a bond to PS, or a linker to PS; wherein the PS is covalently linked, optionally through a linker, to a side chain of a linking amino acid residue, X′ or X.

### Embodiment 28

The compound of any one of embodiments 25 to 27, wherein the PS has a mass of 30-80 kDa.

### Embodiment 29

The compound of any one of embodiments 25 to 28, wherein the PS has a mass of 35-60 kDa.

### Embodiment 30

The compound of any one of embodiments 25 to 29, wherein the PS has a mass of about 40 kDa.

### Embodiment 31

A pharmaceutical composition comprising a compound according to any one of embodiments 25 to 30 in combination with a pharmaceutically acceptable excipient.

### Embodiment 32

A method for treating a disease or disorder in a subject, comprising administering a compound according to any one of embodiments 25 to 31 to a subject in need thereof in an amount effective to treat the disease or disorder.

### Embodiment 33

The method according to embodiment 32, wherein the disease or disorder is diabetes, type 1 diabetes, type 2 diabetes, obesity, hypertension, atherosclerosis, dyslipidemia, congestive heart failure, stroke, hypercholesterolemia, cardiovascular disease, myocardial ischemia, myocardial reperfusion, an eating disorder, gestational diabetes, diabetic neuropathy, pulmonary hypertension or insufficient pancreatic beta cell mass.

### Embodiment 34

The method according to embodiment 33, wherein the disease or disorder is diabetes, type 1 diabetes, type 2 diabetes or gestational diabetes.

### Embodiment 35

The method according to embodiment 33, wherein the disease or disorder is obesity.

### Embodiment 36

The method according to embodiment 33, wherein the disease or disorder is hypertension, atherosclerosis, congestive heart failure, stroke, cardiovascular disease, myocardial ischemia, myocardial reperfusion or pulmonary hypertension.

### Embodiment 37

The method according to embodiment 33, wherein the disease or disorder is dyslipidemia or hypercholesterolemia.

### Embodiment 38

The method or use according to any one of the above embodiments wherein the disease or disorder is diabetes, type 1 diabetes, type 2 diabetes, hypertension, atherosclerosis, dyslipidemia, congestive heart failure, stroke, hypercholesterolemia, cardiovascular disease, myocardial ischemia, myocardial reperfusion, gestational diabetes, diabetic neuropathy, pulmonary hypertension or insufficient pancreatic beta cell mass, and the subject in need thereof is overweight, obese, extremely obese or in need of body weight reduction.

### Embodiment 39

The method or use according to the preceding embodiment, wherein the disease or disorder is diabetes, type 2 diabetes, diabetic neuropathy or insufficient pancreatic beta cell mass, and the subject in need thereof is overweight, obese, extremely obese or in need of body weight reduction.

### Embodiment 40

The compound, composition or method of any one of the above embodiments wherein the P1 or the exendin analog is [Leu¹⁴]exendin-4 or [Leu¹⁴, Lys⁴⁰]exendin-4.

### Embodiment 41

The compound, composition or method of any one of the above embodiments wherein the P2 or the amylin analog is davalitide or [des-Lys¹]-davalintide.

### Embodiment 42

The compound, composition or method of any one of the above embodiments wherein the PS is a polyethylene glycol or a derivative thereof, and the polyethylene glycol or a derivative thereof is linear.

### Embodiment 43

The compound, composition or method of any one of the above embodiments wherein the compound or polypeptide conjugate is Cmpd 14 with structure

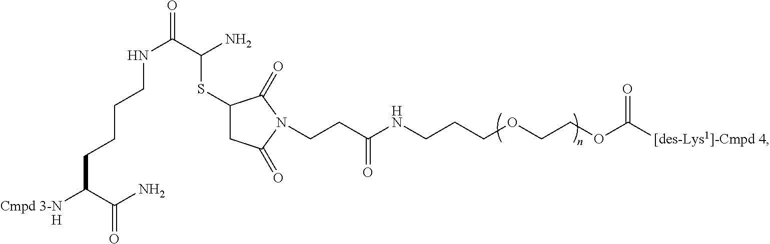

wherein Cmpd 3 is [Leu¹⁴]Exendin-4 (SEQ ID NO:7); Cmpd 4 is davalintide (SEQ ID NO:34); and n is about 900.

### Embodiment 44

The method of any one of embodiments 19 to 24 or 32 to 43, wherein the compound is administered at a daily dose of 1 μg to 1000 μg or at a weekly dose of 7 μg to 7000 μg.

### Embodiment 45

The method of the preceding embodiment wherein the compound is administered at a daily dose of 10 μg to 500 μg or at a weekly dose of 70 μg to 3500 μg.

### Embodiment 46

The method of the preceding embodiment wherein the compound is administered at a daily dose of 50 μg to 500 μg or at a weekly dose of 350 μg to 3500 μg.

### Embodiment 48

The method of the preceding embodiment wherein the compound is administered at a daily dose of 100 μg to 400 μg or at a weekly dose of 100 μg to 350 μg.

It is expressly contemplated and stated that for each and every composition, formulation, mode of administration, administration regimen, method of use, method of treatment or of prevention, combination therapy, method of making, method of use, or any other composition, method, or use or other embodiment described and disclosed in this application, the most preferred compound or polypeptide conjugate is Cmpd 14.

What is claimed is:

1. A long-duration dual hormone conjugate (LDDHC) compound having the structure of Formula I
P₁-L₁-PS-L₂-P₂ (I)
wherein
P₁ is a peptide hormone having a first biological activity;
P₂ is a peptide hormone having a second biological activity;
L₁ and L₂ are independently a bond or a linker; and
PS is a water-soluble polymeric spacer having a molecular weight in the range 30-80 kDa;
wherein
said compound exhibits said first biological activity in a biological assay, and
said compound exhibits said second biological activity in a biological assay.

2. The compound according to claim 1, wherein said P₁ is an exendin, an exendin analog or a derivative thereof.

3. The compound of claim 1, wherein said P₂ is an amylin, pramlintide, davalintide, or an analog or a derivative thereof.

4. The compound of claim 1, wherein said exendin, exendin analog or derivative thereof is exendin-4, exendin-4 analog or derivative thereof.

5. The compound of claim 1, wherein said P₁ is [Leu¹⁴]exendin-4 or [Leu¹⁴,Lys⁴⁰]exendin-4.

6. The compound according to claim 1, wherein said P₁ comprises 1 to 28 residues.

7. The compound of claim 1, wherein P₂ comprises an amino acid sequence of residues 1-37 (SEQ ID NO:31) of Formula (II):
X′-Xaa¹-Cys²-Asn³-Thr⁴-Ala⁵-Thr⁶-Cys⁷-Ala⁸-Thr⁹-
Gln¹⁰-Arg¹¹-Leu¹²-Ala¹³-Asn¹⁴-Phe¹⁵-Leu¹⁶-Val¹⁷-
His¹⁸-Ser¹⁹-Ser²⁰-Xaa²¹-Asn²²-Phe²³-Xaa²⁴-Xaa²⁵-
Xaa²⁶-Xaa²⁷-Xaa²⁸-Xaa²⁹-Thr³⁰-Xaa³¹-Val³²-Gly³³-
Ser³⁴-Asn³⁵-Thr³⁶-Tyr³⁷-X
wherein up to 55% of the amino acids set forth in Formula (II) may be deleted or substituted with a different amino acid; wherein
X′ is hydrogen, an N-terminal capping group, a bond to PS, or a linker to PS;
Xaa′ is Lys or a bond;
Xaa²¹ is Lys, Cys, or Asn;
Xaa²⁴ is Lys, Cys, or Gly;
Xaa²⁵ is Lys, Cys, or Pro;
Xaa²⁶ is Lys, Cys, or Ile;
Xaa²⁷ is Lys, Cys, or Leu;
Xaa²⁸ is Lys, Cys, or Pro;
Xaa²⁹ is Lys, Cys, or Pro;
Xaa³¹ is Lys, Cys, or Asn; and
X is optional, and if present is substituted or unsubstituted amino, substituted or unsubstituted alkylamino, substituted or unsubstituted dialkylamino, substituted or unsubstituted cycloalkylamino, substituted or unsubstituted arylamino, substituted or unsubstituted aralkylamino, substituted or unsubstituted alkyloxy, substituted or unsubstituted aryloxy, substituted or unsubstituted aralkyloxy, hydroxy 1, a bond to PS, or a linker to PS;
wherein said PS is covalently linked, optionally through a linker, to a side chain of a linking amino acid residue, X′ or X.

8. The compound according to claim 7, wherein said linking amino acid residue is cysteine or lysine.

9. The compound of claim 7, wherein said PS is attached to the side chain of the amino acid at position 11, 24-29, or 31 of the compound comprising the structure of Formula (II).

10. The compound of claim 1, wherein said P₂ is davalintide or [des-Lys¹]-davalintide.

11. The compound of claim 1, wherein said compound is Cmpd 14 with structure 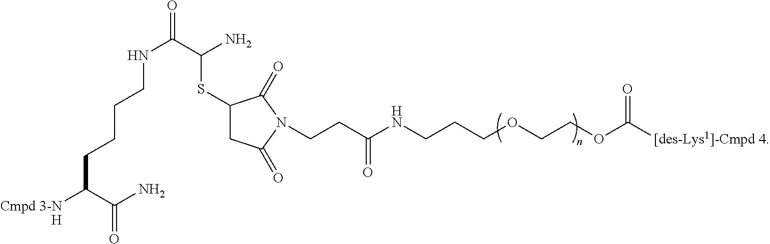
wherein
Cmpd 3 is [Leu¹⁴]Exendin-4 (SEQ ID NO:7),
Cmpd 4 is davalintide (SEQ ID NO:34); and
n is about 900.

12. (canceled)

13. The compound of claim 1, wherein said PS is a polyethylene glycol or a derivative thereof.

14. The compound of claim 13, wherein said polyethylene glycol is linear, branched or comb type.

15-17. (canceled)

18. A pharmaceutical composition comprising a compound according to claim 1 in combination with a pharmaceutically acceptable excipient.

19. A method for treating a disease or disorder in a subject, the method comprising administering a polypeptide conjugate according to claim 1 to a subject in need thereof in an amount effective to treat said disease or disorder.

20. The method according to claim 19, wherein said disease or disorder is diabetes, type 1 diabetes, type 2 diabetes, obesity, hypertension, atherosclerosis, dyslipidemia, congestive heart failure, stroke, hypercholesterolemia, cardiovascular disease, myocardial ischemia, myocardial reperfusion, an eating disorder, gestational diabetes, diabetic neuropathy, pulmonary hypertension or insufficient pancreatic beta cell mass.

21-43. (canceled)

44. The method of claim 19, wherein said compound is administered at a daily dose of 1 μg to 1000 μg or at a weekly dose of 7 μg to 7000 μg.

45-47. (canceled)
